(19) United States
(12) Patent Application Publication
(10) Pub. No.: US 2026/0258409 A1
(43) Pub. Date: Sep. 3, 2026

(54) NUCLEOTIDE SUBSTITUTE HAVING ENHANCED STABILITY

(71) Applicant: Rona Bioscience, Limited, Hong Kong (CN)

(72) Inventors: Jinyu Huang, Shanghai (CN); Hao Zou, Shanghai (CN); Hongli Guo, Shanghai (CN)

(73) Assignee: Rona Bioscience, Limited, Hong Kong (CN)

(21) Appl. No.: 18/878,934

(22) PCT Filed: Jun. 26, 2023
(86) PCT No.: PCT/CN2023/102402

§ 371 Date: Dec. 26, 2024

(30) Foreign Application Priority Data
Jun. 27, 2022 (CN) 202210744263.8
Jul. 21, 2022 (CN) 202210864592.6
Apr. 17, 2023 (CN) 202310411631.1

(51) Int. Cl. C12N 15/113; A61K 31/7125; A61K 31/713; C07F 9/6533

(52) U.S. Cl. CPC C12N 15/113; A61K 31/7125; A61K 31/713; C07F 9/6533; C12N 2310/14; C12N 2310/315; C12N 2310/351; C12N 2310/531

(57) ABSTRACT

Provided are a compound of formula (X), or a pharmaceutically acceptable salt, tautomer or stereoisomer thereof. The compound of formula (X) is connected to the 5′ end and/or the 3′ end of an oligonucleotide, and is used for improving the stability of a double-stranded RNA, or helping double-stranded RNA in resisting exonuclease degradation. The present invention further relates to a double-stranded RNA containing the compound of formula (X), a carrier, a cell, a pharmaceutical composition, and a kit.

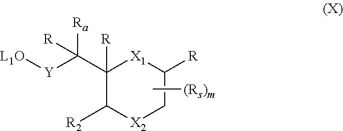

### CROSS REFERENCE TO RELATED APPLICATIONS

This application is a National Stage application under 35 U.S.C. 371 of International PCT Application No. PCT/CN2023/102402, filed on Jun. 26, 2023, which claims priority to the China Patent Application No. 202210744263.8, filed Jun. 27, 2022 and to the China Patent Application No. 202210864592.6 filed on Jul. 21, 2022 and to the China Patent Application No. 202310411631.1, filed Apr. 17, 2023, the disclosure of which applications are incorporated herein by reference in their entirety.

### REFERENCE TO SEQUENCE LISTING

The present application is being filed along with a Sequence Listing in electronic format. The Sequence Listing is provided as a file entitled 145954.04300—Sequence Listing.xml, created on Dec. 23, 2024, which is 33,089 bytes in size. The information in the electronic format of the sequence listing is incorporated herein by reference in its entirety.

### FIELD OF THE INVENTION

The present invention relates to the technical field of medical and pharmaceutical science, specifically as it relates to a nucleotide substitute with enhanced stability for improving the stability of a double-stranded ribonucleic acid (dsRNA) or for helping the dsRNA against exonuclease degradation.

### BACKGROUND OF THE INVENTION

RNA interference refers to a phenomenon of specific and highly efficient degradation of the target mRNA induced by a dsRNA. The efficiency and duration of RNA interference may be influenced since dsRNAs will be degraded by nucleases in the body. The introduction of a nucleotide substitution to a RNA molecule at the end that is most susceptible to nucleases, therefore, may improve the stability of the RNA.

At present, it has been disclosed that molecules based on morpholine ring or dioxane can improve the stability of a dsRNA when the molecule is attached to the 3′ end of the sense strand thereof. However, it is still needed to develop more nucleotide analogues with enhanced stability for the use in the art.

### SUMMARY OF THE INVENTION

In one aspect, the present invention provides a compound of formula (X), or a pharmaceutically acceptable salt, tautomer or stereoisomer thereof:

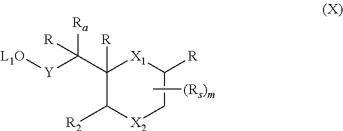

wherein each group is defined as follows.

In another aspect, the present invention provides a compound of formula (I), or a pharmaceutically acceptable salt, tautomer or stereoisomer thereof:

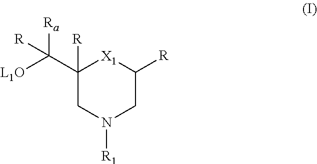

wherein each group is defined as follows.

In another aspect, the present invention provides a compound of formula (Ia), or a pharmaceutically acceptable salt, tautomer or stereoisomer thereof:

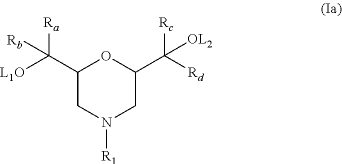

wherein each group is defined as follows.

In another aspect, the present invention provides a compound of formula (Ib), or a pharmaceutically acceptable salt, tautomer or stereoisomer thereof:

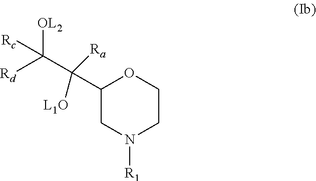

wherein each group is defined as follows.

In another aspect, the present invention provides a compound of formula (Ic), or a pharmaceutically acceptable salt, tautomer or stereoisomer thereof:

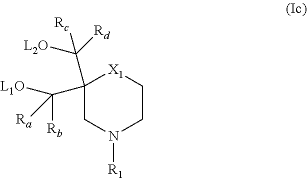

wherein each group is defined as follows.

In another aspect, the present invention provides a compound of formula (II), or a pharmaceutically acceptable salt, tautomer or stereoisomer thereof:

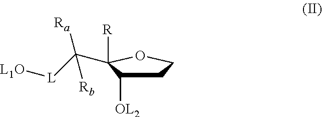

wherein each group is defined as follows.

In another aspect, the present invention provides an oligonucleotide comprising at the 5′ end and/or the 3′ end one or more compounds of formula (X′):

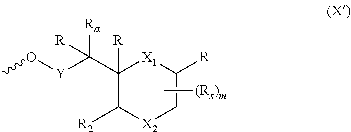

wherein each group is defined as follows.

In another aspect, the present invention provides an oligonucleotide comprising at the 5′ end and/or the 3′ end one or more compounds of formula (I′):

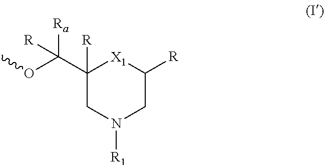

wherein each group is defined as follows.

In another aspect, the present invention provides an oligonucleotide comprising at the 5′ end and/or the 3′ end one or more compounds of formula (Ia′):

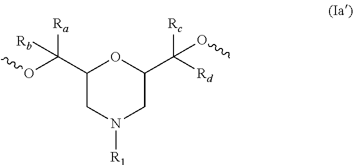

wherein each group is defined as follows.

In another aspect, the present invention provides an oligonucleotide comprising at the 5′ end and/or the 3′ end one or more compounds of formula (Ib′):

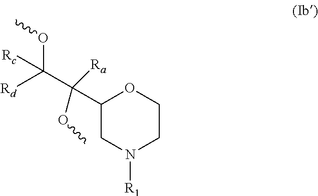

wherein each group is defined as follows.

In another aspect, the present invention provides an oligonucleotide comprising at the 5′ end and/or the 3′ end one or more compounds of formula (Ic′):

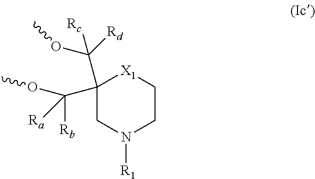

wherein each group is defined as follows.

In another aspect, the present invention provides an oligonucleotide comprising at the 5′ end and/or the 3′ end one or more compounds of formula (II):

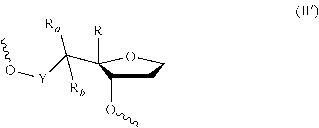

wherein each group is defined as follows.

In another aspect, the present invention provides a dsRNA with a sense strand and an antisense strand between which the sequences are substantially complementary, wherein each strand has 14 to 30 nucleotides that are connected via a phosphate group, a phosphorothioate group or other linker moieties, and wherein said sense strand has the structure as follows:

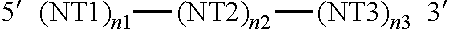

wherein each group is defined as follows.

In another aspect, the present invention provides a vector comprising a nucleotide sequence that encodes the aforementioned dsRNA.

In another aspect, the present invention provides a cell comprising the aforementioned dsRNA or the aforementioned vector.

In another aspect, the present invention provides a pharmaceutical composition comprising the aforementioned dsRNA, the aforementioned vector, or the aforementioned cell, and optionally a pharmaceutically acceptable vehicle or excipient.

In another aspect, the present invention provides a kit comprising the aforementioned dsRNA, the aforementioned vector, or the aforementioned cell.

A compound of the present invention may be attached to either the 3′ end or the 5′ end of a sense strand in order to enhance RNA duplex stability. In addition, a compound of the present invention containing an amino group (including N-containing heterocyclic ring) may be attached to the 5′ end or the 3′ end of a sense strand to enhance RNA duplex stability and to prevent misloading of the sense strand into Ago2 protein.

Without being bound by theory, it is reported by Kumar et al (Chem. Commun., 2019, 55, 5139-5142) that cations would repulsively interact with Lys-570 and Lys-533 in the MID region of Ago2, resulting in a nucleotide strand containing a cation at the 5′ end not being loaded into Ago2. Therefore, the introduction of substituted amines at the 5′ end of a sense strand can form cationic structures and thus can be used to reduce off-target effects caused by misloading of the sense strand into Ago2 or to enhance loading of the antisense strand.

### DETAILED DESCRIPTION OF THE INVENTION

### Definitions

### Chemical Definition

Definitions of specific functional groups and chemical terms are described in more detail as follows.

When a numerical range is provided, it is intended that a particular numerical point and sub-range within said range be included. For example, “C₁₋₆ alkyl” includes alkyls C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆;

“C₁₋₂₀ alkyl” refers to any straight-chain or branched hydrocarbon group being saturated and with 1 to 20 carbon atoms. In some embodiments, C₁₋₆ alkyl, C₁₋₄ alkyl and C₁₋₂ alkyl are preferred. Examples of C₁₋₆ alkyl described herein include, but are not limited to: methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes any heteroalkyl in which one or more (e.g., 1, 2, 3, or 4) carbon atoms are replaced with heteroatoms (e.g., oxygen, sulfur, nitrogen, boron, silicon, phosphorus). The alkyls may be optionally substituted by one or more substituents, for example, 1 to 5 substituents, 1 to 3 substituents, or 1 substituent. The conventional abbreviations for alkyl include: Me(—CH₃), Et(—CH₂CH₃), iPr(—CH(CH₃)₂), nPr(—CH₂CH₂CH₃), n-Bu(—CH₂CH₂CH₂CH₃) or i-Bu(—CH₂CH(CH₃)₂).

“C₂₋₂₀ alkenyl” refers to a straight-chain or branched hydrocarbon group with 2 to 20 carbon atoms and at least one carbon-carbon double bond. In some embodiments, C₂₋₆ alkenyl and C₂₋₄ alkenyl are preferred. Examples of C₂₋₆ alkenyl include, but are not limited to: vinyl (C₂), 1-propenyl (C₃), 2-propenyl (C₃), 1-butenyl (C₄), 2-butenyl (C₄), butadienyl (C₄), pentenyl (C₅), pentadienyl (C₅), and hexenyl (C₆). The term “C₂₋₆ alkenyl” also includes any heteroalkenyl in which one or more (e.g., 1, 2, 3, or 4) carbon atoms are replaced with heteroatoms (e.g., oxygen, sulfur, nitrogen, boron, silicon, phosphorus). The alkenyls may be optionally substituted by one or more substituents, for example, 1 to 5 substituents, 1 to 3 substituents, or 1 substituent.

“C₂₋₂₀ alkynyl” refers to a straight-chain or branched hydrocarbon group with 2 to 20 carbon atoms and at least one carbon-carbon triple bond and optionally one or more carbon-carbon double bonds. In some embodiments, C₂₋₆ alkynyl and C₂₋₄ alkynyl are preferred. Examples of C₂₋₆ alkynyl include, but are not limited to: ethynyl (C₂), 1-propynyl (C₃), 2-propynyl (C₃), 1-butynyl (C₄), 2-butynyl (C₄), pentynyl (C₅), and hexynyl (C₆). The term “C₂₋₆ alkynyl” also includes any heteroalkynyl in which one or more (e.g., 1, 2, 3, or 4) carbon atoms are replaced with heteroatoms (e.g., oxygen, sulfur, nitrogen, boron, silicon, phosphorus). The alkynyls may be optionally substituted by one or more substituents, for example, 1 to 5 substituents, 1 to 3 substituents, or 1 substituent.

“C₁₋₂₀ alkylene” and “C₁₋₆ alkylene”, respectively, refer to a divalent group formed by removal of a hydrogen from a C₁₋₂₀ alkyl and a C₁₋₆ alkyl and by substitution or not by substitution. In some embodiments, C₁₋₄ alkylene, C₂₋₄ alkylene and C₁₋₃ alkylene are preferred. The unsubstituted alkylenes include, but are not limited to: methylene group (—CH₂—), ethylene group (—CH₂CH₂—), propylene group (—CH₂CH₂CH₂—), butylene group (—CH₂CH₂CH₂CH₂—), pentylene group (—CH₂CH₂CH₂CH₂CH₂—), and hexylene group (—CH₂CH₂CH₂CH₂CH₂CH₂—). Examples of said substituted alkylenes, such as a alkylene substituted by one or more alkyl (methyl) groups, include but are not limited to: substituted methylene group (—CH(CH₃)—, and —C(CH₃)₂—), substituted ethylene group (—CH(CH₃)CH₂—, —CH₂CH(CH₃)—, —C(CH₃)₂CH₂—, and —CH_{2C}(CH₃)₂—), substituted propylene group (—CH(CH₃)CH₂CH₂—, ~CH₂CH(CH₃)CH₂—, —CH₂CH₂CH(CH₃)—, —C(CH₃)₂CH₂CH₂—, —CH_{2C}(CH₃)₂CH₂—, and —CH₂CH_{2C}(CH₃)₂—).

“C₀₋₆ alkylene” refers to a chemical bond as well as the aforementioned “C₁₋₆ alkylene”.

“Halo-” or “halogen” refers to (substitution by) fluorine (F), chlorine (Cl), bromine (Br) and iodine (I).

Accordingly, “C₁₋₂₀ haloalkyl”, “C₁₋₆ haloalkyl” and “C₁₋₄ haloalkyl” refer to an aforementioned substituted “C₁₋₂₀ alkyl”, “C₁₋₆ alkyl” and “C₁₋₄ alkyl”, respectively, with one or more halogen groups. In some embodiments, a C₁₋₄ haloalkyl is particularly preferred, and a C₁₋₂ haloalkyl is even more preferred. Exemplary haloalkyls include, but are not limited to: —CF₃, —CH₂F, —CHF₂, —CHFCH₂F, —CH₂CHF₂, ~CF₂CF₃, —CCl₃, —CH₂Cl, —CHCl₂, and 2,2,2-trifluoro-1,1-dimethyl-ethyl. The haloalkyls may be substituted at any substitutable connection site, for example 1 to 5 substituents, 1 to 3 substituents or 1 substituent.

“C₃₋₈ cycloalkyl” refers to a non-aromatic cyclic hydrocarbon group with 3 to 8 ring carbon atoms and no heteroatoms. In some embodiments, C₃₋₇ cycloalkyl and C₃₋₆ cycloalkyl are particularly preferred, and C₄₋₆ cycloalkyl and C₅₋₆ cycloalkyl are even more preferred. A cycloalkyl herein also refers to a ring system in which an aforementioned cycloalkyl ring is fused with one or more aryls or heteroaryls through any connection site(s) on the cycloalkyl ring; in this context, the number of carbons still represents the number of carbons in the cycloalkyl system. Examples of said cycloalkyls include, but are not limited to: cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), and cycloheptatrienyl (C₇). The cycloalkyls may be optionally substituted by one or more substituents, for example, 1 to 5 substituents, 1 to 3 substituents, or 1 substituent.

The term herein “3- to 10-membered heterocyclyl” refers to a saturated or unsaturated group of a 3- to 10-membered non-aromatic ring system with ring carbon atom(s) and 1 to 5 ring heteroatoms, wherein each heteroatom is independently selected from nitrogen, oxygen, sulfur, boron, phosphorus and silicon. In said heterocyclyl containing one or more nitrogen atoms, the connection site may be a carbon or nitrogen atom as long as the valence permits. In some embodiments, a 4- to 10-membered heterocyclyl is preferred, wherein said heterocyclyl is a 4- to 10-membered non-aromatic ring system with ring carbon atom(s) and 1 to 5 ring heteroatoms; in some embodiments, a 3- to 8-membered heterocyclyl is preferred, wherein said heterocycloyl is a 3- to 8-membered non-aromatic ring system with ring carbon atom(s) and 1 to 4 ring heteroatoms; preferably a 4- to 8-membered heterocyclyl as a 4- to 8-membered non-aromatic ring system with ring carbon atom(s) and 1 to 3 ring heteroatoms; preferably a 4- to 7-membered heterocyclyl as a 4- to 7-membered non-aromatic ring system with ring carbon atom(s) and 1 to 3 ring heteroatoms; preferably a 4- to 6-membered heterocyclyl as a 4- to 6-membered non-aromatic ring system with ring carbon atom(s) and 1 to 3 ring heteroatoms; more preferably a 5- to 6-membered heterocyclyl as a 5- to 6-membered non-aromatic ring system with ring carbon atom(s) and 1 to 3 ring heteroatoms. A heterocyclyl herein also includes a ring system in which an aforementioned heterocyclyl ring is fused to one or more cycloalkyls through any connection site(s) on the cycloalkyl ring, or said heterocyclyl includes a ring system in which an aforementioned heterocyclyl ring is fused to one or more aryls or heteroaryls through any connection site(s) on the heterocyclyl ring; in these contexts, the number of ring members still represents the number of ring members in the heterocyclyl ring system. Exemplary 3-membered heterocyclyls containing one heteroatom include, but are not limited to: aziridinyl, oxiranyl, and thiorenyl. Exemplary 4-membered heterocyclyls containing one heteroatom include, but are not limited to: azetidinyl, oxetidinyl, and thietanyl. Exemplary 5-membered heterocyclyls containing one heteroatom include, but are not limited to: tetrahydrofuryl, dihydrofuryl, tetrahydrothienyl, dihydrothienyl, pyrrolidinyl, dihydropyrrolyl, and pyrroli-2,5-dione. Exemplary 5-membered heterocyclyls containing two heteroatoms include, but are not limited to: pyrazolidinyl, dioxolanyl, oxasulfuranyl, disulfuranyl, and oxazolidin-2-one. Exemplary 5-membered heterocyclyls containing three heteroatoms include, but are not limited to: triazolinyl, oxadiazolinyl and thiadiazolinyl. Exemplary 6-membered heterocyclyls containing one heteroatom include, but are not limited to: piperidinyl, tetrahydropyranyl, dihydropyridyl and thianyl. Exemplary 6-membered heterocyclyls containing two heteroatoms include, but are not limited to: piperazinyl, morpholinyl, dithianyl, and dioxanyl. Exemplary 6-membered heterocyclyls containing three heteroatoms include, but are not limited to: triazinanyl. Exemplary 7-membered heterocyclyls containing one heteroatom include, but are not limited to: azepanyl, oxepanyl, and thiepanyl. Exemplary 5-membered heterocyclyls each of which is fused with a C₆ aryl ring (also referred to herein as a 5,6-bicycloheterocyclyl) include, but are not limited to: dihydroindolyl, isodihydroindolyl, dihydrobenzofuryl, dihydrobenzothienyl, and benzoxazolinonyl. Exemplary 6-membered heterocyclyls each of which is fused with a C6 aryl ring (also referred to herein as a 6,6-bicycloheterocyclyl) include, but are not limited to: tetrahydroquinolinyl, and tetrahydroisoquinolinyl. A heterocyclyl herein also includes any bridged ring or spiro ring in which an aforementioned heterocyclyl shares one or two atoms with a cycloalkyl, heterocyclyl, aryl or heteroaryl, and the shared atom(s) may be carbon or nitrogen as long as the valence permits. The heterocyclyls also include any ones of an aforementioned heterocycly that is optionally substituted by one or more substituents, for example, 1 to 5 substituents, 1 to 3 substituents, or 1 substituent.

“C₆₋₁₀ aryl” refers to a monocyclic or polycyclic (e.g., bicyclic) group that is a 4n+2 aromatic ring system having 6 to 10 ring carbon atoms and no heteroatom (e.g., with 6 or 10π electrons shared in a cyclic arrangement). In some embodiments, an aryl has six ring carbon atoms (“C₆ aryl”; e.g., phenyl). In some embodiments, an aryl has ten ring carbon atoms (“C₁₀ aryl”; e.g., a naphthyl, such as 1-naphthyl and 2-naphthyl). An aryl herein also includes a ring system in which an aforementioned aryl ring is fused with one or more cycloalkyls or heterocyclyls through the connection sites on said aryl ring; in this context, the number of carbon atoms still represents the number of carbon atoms in said aryl ring system. The aryls may be optionally substituted by one or more substituents, for example, 1 to 5 substituents, 1 to 3 substituents, or 1 substituent.

“5- to 14-membered heteroaryl” refers to a 5- to 14-membered monocyclic or bicyclic group of a 4n+2 aromatic ring system (e.g., with 6, 10 or 14π electrons shared in a cyclic arrangement) that has ring carbon atom(s) and 1 to 4 ring heteroatoms, wherein each heteroatom is independently selected from nitrogen, oxygen and sulfur. In said heteroaryl containing one or more nitrogen atoms, the connection site may be a carbon or nitrogen atom as long as the valence permits. A bicyclic heteroaryl system herein may comprise one or more heteroatoms in one or both rings thereof. A heteroaryl herein also includes a ring system in which an aforementioned heteroaryl ring is fused with one or more cycloalkyls or heterocyclyls through the connection sites on said heteroaryl ring; in this context, the number of carbon atoms still represents the number of carbon atoms in said heteroaryl ring system. In some embodiments, a 5- to 10-membered heteroaryl is preferred, wherein said heteroaryl is a 4n+2 aromatic ring system of a 5- to 10-membered monocyclic or bicyclic ring with ring carbon atom(s) and 1 to 4 ring heteroatoms. In some other embodiments, a 5- to 6-membered heteroaryl is particularly preferred, wherein said heteroaryl is a 4n+2 aromatic ring system of a 5- to 6-membered monocyclic or bicyclic ring with ring carbon atom(s) and 1 to 4 ring heteroatoms. Exemplary 5-membered heteroaryls containing one heteroatom include, but are not limited to: pyrrolyl, furyl, and thienyl. Exemplary 5-membered heteroaryls containing two heteroatoms include, but are not limited to: imidazolyl, pyrazolyl, oxazolyl, isoxazolyl, thiazolyl, and isothiazolyl. Exemplary 5-membered heteroaryls containing three heteroatoms include, but are not limited to: triazolyl, oxadiazolyl (e.g., 1,2,4-oxadiazolyl), and thiadiazolyl. Exemplary 5-membered heteroaryls containing four heteroatoms include, but are not limited to: tetrazolyl. Exemplary 6-membered heteroaryls containing one heteroatom include, but are not limited to: pyridinyl or pyridinonyl. Exemplary 6-membered heteroaryls containing two heteroatoms include, but are not limited to: pyridazinyl, pyrimidinyl, and pyrazinyl. Exemplary 6-membered heteroaryls containing three or four heteroatoms include, but are not limited to: triazinyl, and tetrazinyl. Exemplary 7-membered heteroaryls containing one heteroatom include, but are not limited to: azepinyl, oxepinyl, and thiepinyl. Exemplary 5,6-bicycloheteroaryls include, but are not limited to: indolyl, isoindolyl, indazolyl, benzotriazolyl, benzothienyl, isobenzothienyl, benzofuryl, benzoisofuryl, benzimidazolyl, benzoxazolyl, benzisoxazolyl, benzooxadiazolyl, benzothiazolyl, benzoisothiazolyl, benzothiadiazolyl, indolizinyl, and purinyl. Exemplary 6,6-bicycloheteroaryls include, but are not limited to: naphthalidinyl, pteridinyl, quinolyl, isoquinolyl, cinnolinyl, quinoxalinyl, phthalazinyl, and quinazolinyl. The heteroaryls may be optionally substituted by one or more substituents, for example, 1 to 5 substituents, 1 to 3 substituents, or 1 substituent.

Divalent groups formed by the removal of a hydrogen from such groups as alkyl, alkenyl, alkynyl, cycloalkyl, heterocyclyl, aryl and heteroaryl as defined above are collectively referred to as “-ylene group” herein. The ring-forming groups such as cycloalkyl, heterocyclyl, aryl and heteroaryl are collectively referred to as “cyclyl”

Alkyl, alkenyl, alkynyl, cycloalkyl, heterocyclyl, aryl and heteroaryl as defined herein are optionally substituted groups.

Exemplary substituents on carbon atoms include, but are not limited to: halogen, —CN, —NO₂, —N₃, —SO₂H, —SO₃H, —OH, —OR^{aa}, —ON(R^{bb})₂, —N(R^{bb})₂, —N(R^{bb}) 3 X, —N(OR)R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR)OR^{aa}, —C(═NR^{bb})N(R)₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR)₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR)₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, cycloalkyl, heterocyclyl, aryl and heteroaryl, wherein each of alkyl, alkenyl, alkynyl, cycloalkyl, heterocyclyl, aryl and heteroaryl is independently substituted with 0, 1, 2, 3, 4 or 5 R^{dd} groups;
or wherein two geminal hydrogens at a carbon atom are substituted by a group, such as ═O, ═S, ═NN(R^{bb})₂, ═NNR^{bb}C(═O) R^{aa}, ═NNR^{bb}C(═O)OR^{aa}, ═NNR^{bb}S(═O)₂R^{aa}, ═NR^{bb} or ═NOR^{cc};
wherein each of R^{aa} is independently selected from alkyl, haloalkyl, alkenyl, alkynyl, cycloalkyl, heterocyclyl, aryl and heteroaryl, or two R^{aa} groups are connected each other to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{dd} groups;

Each of R^{bb} is independently selected from: hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR₂, —C(═NR^{cc})N(R^{cc})₂, —SO₂N(R^{cc})₂, —SO₂R^{cc}, —SO₂OR^{cc}, —SOR^{aa}, —C(═S)N(R)₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are connected each other to form a heterocyclyl ring or heteroaryl ring, wherein each alkyl, alkenyl, alkynyl, cycloalkyl, heterocyclyl, aryl and heteroaryl group is independently substituted with 0, 1, 2, 3, 4 or 5 R^{dd} groups;
wherein each of R^{cc} is independently selected from hydrogen, alkyl, haloalkyl, alkenyl, alkynyl, cycloalkyl, heterocyclyl, aryl and heteroaryl, or two R^{cc} groups are connected each other to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{dd} groups;

Each of R^{dd} is independently selected from: halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{aa}, —C(═NR^{ff})N(R^{ff})₂, —OC(═NR^{ff})N(R^{ff})₂, —NR^{ee}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR)₂, alkyl, haloalkyl, alkenyl, alkynyl, cycloalkyl, heterocyclyl, aryl, heteroaryl, wherein each of alkyl, alkenyl, alkynyl, cycloalkyl, heterocyclyl, aryl and heteroaryl is independently substituted with 0, 1, 2, 3, 4 or 5 R^{gg} groups, or two geminal R^{dd} substituents may be combined to form ═O or ═S;

Each of R^{ee} is independently selected from: alkyl, haloalkyl, alkenyl, alkynyl, cycloalkyl, aryl, heterocyclyl and heteroaryl, wherein each alkyl, alkenyl, alkynyl, cycloalkyl, heterocyclyl, aryl and heteroaryl is independently substituted with 0, 1, 2, 3, 4 or 5 R^{gg} groups;
wherein each of R^{ff} is independently selected from hydrogen, alkyl, haloalkyl, alkenyl, alkynyl, cycloalkyl, heterocyclyl, aryl and heteroaryl, or two Rf groups are connected each other to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{gg} groups;

Each of R^{gg} is independently selected from: halogen, —CN, —NO₂, —N₃, —SO₂H, —SO₃H, —OH, —OC₁₋₆Alkyl, —ON(C₁₋₆(Alkyl)₂, —N(C₁₋₆Alkyl)₂, —N(C₁₋₆Alkyl)₃⁺X⁻, —NH(C₁₋₆(Alkyl)₂⁺X, —NH₂(C₁₋₆Alkyl)⁺X⁻, —NH₃⁺X⁻, —N(OC₁₋₆Alkyl)(C₁₋₆Alkyl), —N(OH)(C₁₋₆Alkyl), —NH(OH), —SH, —SC₁₋₆Alkyl, —SS(C₁₋₆Alkyl), —C(═O)(C₁₋₆Alkyl), —CO₂H, —CO₂(C₁₋₆Alkyl), —OC(═O)(C₁₋₆Alkyl), —(C₁₋₆Alkyl), —C(═O)NH₂, —C(═O)N(C₁₋₆Alkyl)₂, —OC(═O)NH(C₁₋₆Alkyl), —NHC(═O)(C₁₋₆(Alkyl), —N(C₁₋₆Alkyl)C(═O)(C₁₋₆Alkyl), —NHCO₂ (C₁₋₆Alkyl), —NHC(═O)N(C₁₋₆Alkyl)₂, —NHC(═O)NH(C₁₋₆Alkyl), —NHC(═O)NH₂, —C(═NH)O(C₁₋₆Alkyl), —OC(═NH)(C₁₋₆Alkyl), —OC(═NH)OC₁₋₆Alkyl, —C(═NH)N(C₁₋₆Alkyl)₂, —C(═NH)NH(C₁₋₆(Alkyl), —C(═NH)NH₂, OC(═NH)N(C₁₋₆Alkyl)₂, —OC(NH)NH(C₁₋₆Alkyl), —OC(NH)NH₂, —NHC(NH)N(C₁₋₆Alkyl)₂, —NHC(═NH)NH₂, —NHSO₂(C₁₋₆Alkyl), —SO₂N(C₁₋₆Alkyl)₂, —SO₂NH(C₁₋₆Alkyl), —SO₂NH₂, —SO₂C₁₋₆Alkyl, —SO₂OC₁₋₆Alkyl, —OSO₂C₁₋₆Alkyl, —SOCl₆Alkyl, —Si(C₁₋₆Alkyl) 3, —OSi(C₁₋₆Alkyl)₃, —C(═S)N(C₁₋₆Alkyl)₂, C(═S)NH(C₁₋₆Alkyl), C(═S)NH₂, —C(═O)S(C₁₋₆Alkyl), —C(═S)SC₁₋₆Alkyl, —SC(═S)SC₁₋₆Alkyl, —P(═O)₂(C₁₋₆Alkyl), —P(═O)(C₁₋₆Alkyl)₂, —OP(═O)(C₁₋₆(Alkyl)₂, —OP(═O)(OC₁₋₆Alkyl)₂, C₁₋₆Alkyl, C₁₋₆Haloalkyl, C₂₋₆Alkenyl, C₂-C₆Alkynyl, C₃-C₇Cycloalkyl, C₆-C₁₀Aryl, C₃-C₇Heterocyclyl, C₅-C₁₀Heteroaryl; or two geminal R^{gg} substituents may be connected to form ═O or ═S; wherein, X− is a counterion.

Exemplary substituents on a nitrogen atom include, but are not limited to: hydrogen, —OH, —OR^{aa}, —N(R)₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(RC)₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{cc} groups connecting to said nitrogen atom are connected each other to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described above.

### Some Additional Definitions

The term “siRNA” herein is a class of double-stranded RNA molecules each of which can mediate the silencing of target RNA (e.g., mRNA, e.g., transcript of a gene encoding a protein) complementary thereto. A siRNAs is generally double-stranded, including an antisense strand complementary to the target RNA thereof and a sense strand complementary to this antisense strand. For the sake of convenience, such an mRNA is also referred to herein as mRNA to be silenced, and such a gene is also called target gene. Usually, a RNA to be silenced herein is an endogenous gene or a pathogen gene. In addition, RNAs other than mRNA (e.g. tRNA) as well as viral RNA may also be targeted.

The term “antisense strand” herein refers to a strand of a siRNA, wherein said strand contains a region that is completely or substantially complementary to the target sequence thereof. The term “sense strand” herein refers to a strand of a siRNA, wherein said strand contains a region that substantially complementary to a region of an antisense strand as defined herein.

The term “complementary region” herein refers to a region on an antisense strand that is completely or substantially complementary to the target mRNA sequence thereof. In cases where a complementary region is incompletely complementary to the target sequence thereof, a mismatch may be located in an internal or terminal region of the molecule. Typically, a mismatch most tolerant is located in a terminal region, e.g., within 5, 4, 3, 2 or 1 nucleotide at the 5′ and/or 3′ end. A region in an antisense strand, which is most sensitive to mismatch, is called “seed region”. For example, in a siRNA containing a strand of 19 nt, the 19th site (counting from the 5′ end to the 3′ end) can tolerate some mismatches.

The term “complementary” refers to the ability of a first polynucleotide to hybridize with a second polynucleotide under certain conditions, such as stringent conditions. For example, stringent conditions may include 400 mM NaCl, 40 mM PIPES pH 6.4, 1 mM EDTA, and 50° C. or 70° C. for 12-16 hours. With respect to fulfilling the above required capabilities related to the hybridization ability thereof, said “complementary” sequences may also include or be entirely composed of non-Watson-Crick base pairs and/or base pairs formed from non-natural as well as modified nucleotides. Such non-Watson-Crick base pairs include, but are not limited to, G: U wobble base pairing or Hoogsteen base pairing.

A polynucleotide that is “at least partially complementary” or “substantially complementary” to a messenger RNA (mRNA) refers to a polynucleotide that is substantially complementary to a continuous portion of the mRNA of interest. For example, a polynucleotide is at least partially complementary to an mRNA encoding PCSK9, when the sequence thereof is substantially complementary to an uninterrupted portion of said PCSK9 mRNA. The terms “complementary,” “completely complementary,” and “substantially complementary” as used herein may be applied to base pairing between the sense strand and antisense strand of a siRNA, or between the antisense strand of a siRNA reagent and the target sequence thereof.

The term “shRNA” herein refers to short hairpin RNA. An shRNA comprises two short inverted repeat sequences. An shRNA cloned into an shRNA expression vector comprises of two short inverted repeat sequences, separated by a loop sequence, forming a hairpin structure and controlled by the RNA polymerase III (pol III) promoter. Subsequently, 5 to 6 Ts are ligated as transcription terminators of pol III.

“Nucleoside” refers to a compound comprising two substances: one is a purine base or a pyrimidine base, and the other is a ribose or a deoxyribose; “nucleotide” refers to a compound comprising three substances: one is a purine base or a pyrimidine base, another is a ribose or deoxyribose, and the third is a phosphoric acid; and “oligonucleotide” refers to, for example, a nucleic acid molecule (RNA or DNA) with a length of less than 100, 200, 300 or 400 nucleotides.

The term “base” refers to a fundamental building block of nucleosides, nucleotides and nucleic acids; as always containing nitrogen, said base is also referred to as “nitrogenous base.” Unless otherwise specified, the capital letters herein, i.e., A, U, T, G and C, denote the bases of nucleotides, which refers to adenine, uracil, thymine, guanine and cytosine, respectively.

As used herein, the “modification” of nucleotides includes, but is not limited to: methoxyl substitution (methoxy-modified), fluorine substitution (fluoro-modified), connection with a phosphorothioate group, or protection with a conventional protecting group. For example, a fluoro-modified nucleotide refers to a nucleotide formed by substituting the hydroxyl at the 2′ position of the ribosyl of the nucleotide with a fluorine atom, while a methoxy-modified nucleotide refers to a nucleotide formed by substituting the 2′-hydroxyl of the ribosyl with a methoxyl.

“Modified nucleotides” herein include, but are not limited to: a 2′-O-methyl modified nucleotide, a 2′-fluoro modified nucleotide, a 2′-deoxy-modified nucleotide, an inosine ribonucleotide, an abasic nucleotide, an inverted abasic deoxyribonucleotide, a nucleotide comprising a phosphorothioate group, a nucleotide modified by vinylphosphonate, a locked nucleotide, 2′-amino-modified nucleotide, 2′-alkyl-modified nucleotide, a morpholino nucleotide, a phosphoramidate, a non-natural base comprising nucleotide, and a terminal nucleotide linked to a cholesteryl derivative or a dodecanoic acid bisdecylamide group, deoxyribonucleotide, or a nucleotide with protection of a conventional protecting group. For example, a 2′-fluoro modified nucleotide refers to a nucleotide formed by substituting the hydroxyl at the 2′ position of the ribosyl in a nucleotide with a fluorine atom. Said 2′-deoxy-modified nucleotide refers to a nucleotide formed by substituting the 2′-hydroxyl of the ribosyl with a methoxyl.

The term “ligand moiety” refers to a chemical component conjugated with a siRNA, wherein the moiety is capable of changing the distribution, targeting, or lifespan of the siRNA. In a preferred embodiment, such a ligand offers enhanced affinity for selected targets, such as molecules, cells or cell types, and compartments (e.g., cellular or organ compartments, tissues, organs, or regions of the body), compared to for example a siRNA without such a ligand.

The term “reactive phosphorus group” refers to a phosphorus-containing group included within a nucleotide unit or a nucleotide analogue unit, wherein the group can undergo a nucleophilic attack to react with a hydroxyl or amine group in another molecule, especially another nucleotide unit or nucleotide analogue unit. Typically, such a reaction generates an ester-type internucleoside bond connecting a said first nucleotide unit or a said first nucleotide analogue unit with a said second nucleotide unit or a said second nucleotide analogue unit. A reactive phosphorus group can be selected from phosphoramidite, H-phosphonate, alkyl-phosphonate, phosphate or phosphate mimics, including but not limited to: natural phosphate, phosphorothioate, phosphorodithioate, borano phosphate, borano thiophosphate, phosphonate, halogen substituted phosphonates and phosphates, phosphoramidates, phosphodiester, phosphotriester, thiophosphodiester, thiophosphotriester, diphosphates and triphosphates.

“Protecting group” refers to any atom or group of atoms added to a molecule to prevent undesired chemical reactions of existing groups within the molecule. A “protecting group” may be an unstable chemical moiety known in the art, which is used to protect reactive groups such as hydroxyl, amino and thiol groups to prevent undesired or premature reactions during chemical synthesis. Protecting groups are typically used selectively and/or orthogonally to protect sites during the reactions of other reactive sites, which can then be removed to leave the unprotected groups intact or available for further reactions.

A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl) ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Preferred protecting groups are selected from acetyl (Ac), benzoyl (Bzl), benzyl (Bn), isobutyryl (iBu), phenylacetyl, benzyloxymethyl acetal (BOM), beta-methoxyethoxymethyl ether (MEM), methoxymethylether (MOM), p-methoxybenzyl ether (PMB), methylthiomethyl ether, pivaloyl (Piv), tetrahydropyranyl (THP), triphenylmethyl (Trt), methoxytrityl [(4-methoxyphenyl)diphenylmethyl-] (MMT), dimethoxytrityl, [bis-(4-methoxyphenyl)phenylmethyl (DMT), trimethylsilyl ether (TMS), tert-butyldimethylsilyl ether (TBDMS), tri-iso-propylsilyloxymethyl ether (TOM), tri-isopropylsilyl ether (TIPS), methyl ethers, ethoxyethyl ethers (EE) N,N-dimethylformamidine and 2-cynaonethyl (CE).

“Hydroxy-protecting group” refers to a group that can prevent a hydroxyl from undergoing chemical reactions and can be removed under specific conditions to restore the hydroxyl. The main hydroxy-protecting groups include silane-type, acyl-type or ether-type protecting groups, preferably the following:
trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM).

As used herein, the term “pharmaceutically acceptable salt” refers to carboxylates or amino acid salts of a compound of the present invention, which are suitable for contact with patient tissues within a scope of sound medical judgment without causing excessive toxicity, irritation, allergic reactions, etc., and are effective in terms of the intended use with a reasonable benefit/risk ratio; said salt includes, where applicable, the zwitterionic form of a compound of the present invention.

The present invention includes tautomers, which are functional-group isomers resulting from the rapid migration of an atom in a molecule between two positions. A compound with different tautomeric forms, said herein, refers to all the tautomers and does not be restricted to any specific tautomeric form.

A compound of the present invention may include one or more asymmetric centers, and thus may exist in various stereoisomeric forms, such as enantiomers and/or diastereomers. For example, a compound of the present invention may be one of the forms of enantiomer, diastercoisomer or geometric isomer (e.g. a cis isomer or a trans isomer), or may be a mixture of any type of stereoisomerism, including a racemic mixture and a mixture enriched with one or more forms of stereoisomer. An isomer herein may be achieved by separating from a mixture via any method known to those skilled in the art, wherein the method includes chiral high-pressure liquid chromatography (HPLC) and the formation and crystallization of chiral salts; alternatively, a preferred isomer may be prepared through asymmetric synthesis.

The present invention also includes isotopically labeled compounds (isotopic variants) which are equivalent to those described by formula (I), except that one or more atoms are replaced with atoms with an atomic mass or mass number different from that common in nature. Examples of isotopes which may be incorporated into a compound of the present invention include isotopes of hydrogen, carbon, nitrogen, oxygen, phosphorus, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl. Any compounds based on a compound of the present invention and containing any aforementioned isotope and/or any isotope of other atoms, the prodrugs thereof and the pharmaceutically acceptable salts of said compounds or said prodrugs all fall in the scope of the present invention. Certain isotopically labeled compounds of the present invention, such as a compound into which any radioisotope (e.g. ³H and ¹⁴C) is introduced, may be used for distribution determinations of a drug and/or the substrate tissue thereof. Tritium, i.e. ³H and carbon-14, i.e. ¹⁴C isotopes are particularly preferred, because they can be easily prepared and detected. Furthermore, substitution with an isotope heavier, such as deuterium, i.e. ²H, may in some cases be preferred because resultant increased metabolic stability may provide therapeutic benefits such as prolonged in vivo half-life or reduced dosage. An isotopically labeled compound of formula (I) of the present invention and the prodrug thereof may generally be prepared with any readily available isotopically labeled reagent instead of any non-isotopically labeled reagent, in a procedure described below and/or in a process disclosed in any of the Examples and Preparations.

### Compounds of the Present Invention

The present invention relates specifically to a compound of formula (X), or a pharmaceutically acceptable salt, tautomer or stereoisomer thereof:

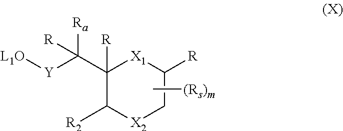

wherein,
X₁ is a bond, O or S;
X₂ is a bond or NR₁;
Y is a bond or [C(Rₐ)(R_{b})]₁-4;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O)NR″R′″, C(S)NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
R₂ is selected from H, D or OL₂;
R is selected from H, D, -L-OR′, -L-NR″R′″, R_{b} or —C(OL₂)(R_{c})(R_{d}), wherein R may be optionally deuterated until fully deuterated;
L₁ and L₂ are selected from H, a reactive phosphorus group or a protecting group;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a bond or C₁₋₆ alkylene, wherein L may be optionally deuterated or fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated or fully deuterated;
n=0, 1, 2, 3, 4, 5, 6, 7, 8, 9 or 10;
p=0 or 1;
Rₛ is selected from H, D, C₁₋₆ alkyl or C₁₋₆ haloalkyl, wherein Rₛ may also be optionally deuterated or fully deuterated;
m=0, 1, 2, 3 or 4;
wherein an OL₂ group is present in either R or R₂.

### X₁

In one embodiment, X₁ is a bond; In another embodiment, X₁ is O. In another embodiment, X₁ is S.

### X₂

In one embodiment, X₂ is a bond; In another embodiment, X₂ is NR₁.

### Y

In one embodiment, Y is a bond; In another embodiment, Y is [C(Rₐ)(R_{b})]₁₋₄, e.g. C(Rₐ)(R_{b}), [C(Rₐ)(R_{b})]₂, [C(Rₐ)(R_{b})]₃ or [C(Rₐ)(R_{b})]₄, preferably C(Rₐ)(R_{b}).

### R₁

In one embodiment, R₁ is C₁₋₂₀ alkyl; In another embodiment, R₁ is C₁₋₂₀ haloalkyl; In another embodiment, R₁ is C₂₋₂₀ alkenyl; In another embodiment, R₁ C₂₋₂₀ alkynyl; In another embodiment, R₁ C₃₋₈ cycloalkyl, preferably C₅₋₆ cycloalkyl; In another embodiment, R₁ is 3- to 10-membered heterocyclyl; In another embodiment, R₁ is C₆₋₁₀ aryl; In another embodiment, R₁ is 5- to 14-membered heteroaryl; In another embodiment, R₁ is —[C(O)]ₚ—C₁₋₂₀ alkylene-(OCH₂CH₂)ₙRₓ; In another embodiment, R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocycl, C₆₋₁₀ aryl or 5- to 14-membered heteroaryl; In another embodiment, R₁ is selected from C₃₋₂ cycloalkyl, 3- to 10-membered heterocyclyl, C₆₋₁₀ aryl or 5- to 14-membered heteroaryl; In another embodiment, R₁ is selected from C₃₋₈ cycloalkyl or 3- to 10-membered heterocyclyl.

In a more specific embodiment, R₁ is optionally substituted with one or more groups selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; In another more specific embodiment, R₁ is optionally substituted with one or more groups selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₅ alkyl or C₁₋₆ haloalkyl; In another more specific embodiment, R¹ is optionally substituted with one or more groups selected from halogen, CN, OR′, SR′, NR″R′″, C₁₋₆ alkyl or C₁₋₆ haloalkyl; In another more specific embodiment, R₁ is optionally deuterated or fully deuterated.

### R₂

In one embodiment, R₂ is H; In another embodiment, R₂ is D. In another embodiment, R₂ is OL₂.

### R

In one embodiment, R is H; In another embodiment, R is D; In another embodiment, R is -L-OR′; In another embodiment, R is -L-NR″R″; In another embodiment, R is R_{b}; In another embodiment, R is —C(OL₂)(R_{c})(R_{d}); In another embodiment, R is selected from H, D, -L-OR′, -L-NR″R″ or R_{b}; In another embodiment, one of the Rs is —C(OL₂)(R_{c})(R_{d}).

In a more specific embodiment, R is optionally deuterated until or deuterated.

L₁ and L₂

In one embodiment, L₁ and L₂ are H; In another embodiment, L₁ and L₂ are reactive phosphorus groups; In another embodiment, L₁ and L₂ are protecting groups; In another embodiment, one of L₁ and L₂ is a reactive phosphorus group and the other is a protecting group.

Rₐ, R_{b}, R_{c} and R_{d}

In one embodiment, Rₐ, R_{b}, R_{c}, and R_{d} are H; In another embodiment, Rₐ, R_{b}, R_{c}, and Rₐ are D; In another embodiment, Rₐ, R_{b}, R_{c}, and R_{d} are halogen; In another embodiment, Rₐ, R_{b}, R_{c}, and R_{d} are CN; In another embodiment, Rₐ, R_{b}, R_{c}, and R_{d} are -L-OR′; In another embodiment, Rₐ, R_{b}, R_{c}, and R_{d} are -L-NR″R″; In another embodiment, Rₐ, R_{b}, R_{c}, and R_{d} are C₁₋₆ alkyl; In another embodiment, Rₐ, R_{b}, R_{c}, and R_{d} are C₁₋₆ haloalkyl; In another embodiment, Rₐ, R_{b}, R_{c}, and R_{d} are C₂₋₆ alkenyl; In another embodiment, Rₐ, R_{b}, R_{c}, and R_{d} are C₂₋₆ alkynyl; In another embodiment, Rₐ, R_{b}, R_{c}, and R_{d} are -L-C₃₋₈ cycloalkyl; In another embodiment, Rₐ, R_{b}, R_{c}, and R_{d} are -L-4- to -L-7-membered heterocyclyl; In another embodiment, Rₐ, R_{b}, R_{c}, and R_{d} are independently selected from H, D, halogen, CN, —C₀₋₆ alkyl-OR′, —C₀₋₆ alkyl-NR″R′″, C₁₋₆ alkyl, C₁₋₆ haloalkyl, C₂₋₆ alkenyl, or C₂₋₆ alkynyl; In another embodiment, Rₐ, R_{b}, R_{c}, and R_{d} are independently selected from H, D, halogen, CN, C₁₋₆ alkyl, or C₁₋₆ haloalkyl.

In a more specific embodiment, Rₐ, R_{b}, R_{c} and R_{d} are optionally deuterated or fully deuterated.

### L

In one embodiment, L is a bond; In another embodiment, L is C₁₋₆ alkylene, and is optionally deuterated or fully deuterated.

### R′, R″ and R′″

In one embodiment, R′, R″ and R′″ are H; In another embodiment, R′, R″, and R″ are —C₁₋₆ alkylene-OH; In another embodiment, R′, R″, and R″ are —C₁₋₆ alkylene-NH₂; In another embodiment, R′, R″, and R″ are C₁₋₆ alkyl; In another embodiment, R′, R″ and R′″ are C₁₋₆ haloalkyl; In another embodiment, R′, R″ and R′″ are C₂₋₆ alkenyl; In another embodiment, R′, R″ and R″ are C₂₋₆ alkynyl; In another embodiment, R′, R″ and R′″ are —C₀₋₆ alkylene-C₃₋₈ cycloalkyl; In another embodiment, R′, R″ and R′″ are —C₀₋₆ alkylene-3-to-C₀₋₆ alkylene-10-membered heterocyclyl; In another embodiment, R′, R″ and R′″ are —C₀₋₆ alkylene-C₆₋₁₀ aryl; In another embodiment, R′, R″ and R′″ are —C₀₋₆ alkylene-5-to-C₀₋₆ alkylene-14-membered heteroaryl; In another embodiment, R″ and R′″ together with the N atom connecting thereto form a 3- to 10-membered heterocyclyl.

In a more specific embodiment, R′, R″ and R′″ are optionally substituted with one or more groups selected from halogen, CN, NO₂, OH, NH₂, C₁₋₆ alkyl, or C₁₋₆ haloalkyl.

### Rₓ

In one embodiment, Rₓ is H; In another embodiment, Rₓ is D; In another embodiment, Rₓ is OH; In another embodiment, Rₓ is OC₁₋₆ alkyl; In another embodiment, Rₓ is C₁₋₆ alkyl; In another embodiment, Rₓ is C₁₋₆ haloalkyl; In another embodiment, Rₓ is C₂₋₆ alkenyl; In another embodiment, Rₓ is C₂₋₆ alkynyl; In another embodiment, Rₓ is C₃₋₈ cycloalkyl; In another embodiment, Rₓ is a 3- to 10-membered heterocyclyl; In another embodiment, Rₓ is C₆₋₁₀ aryl; In another embodiment, Rₓ is a 5- to 14-membered heteroaryl.

In a more specific embodiment, Rₓ is optionally deuterated or fully deuterated.

n

In one embodiment, n is 0; In another embodiment, n is 1; In another embodiment, n is 2; In another embodiment, n is 3; In another embodiment, n is 4; In another embodiment, n is 5; In another embodiment, n is 6; In another embodiment, n is 7; In another embodiment, n is 8; In another embodiment, n is 9; In another embodiment, n is 10.

p

In one embodiment, p is 0; In another embodiment, p is 1.

### R

In one embodiment, Rₛ is H; In another embodiment, Rₛ is D; In another embodiment, Rₛ is C₁₋₆ alkyl; In another embodiment, Rₛ is C₁₋₆ haloalkyl.

In a more specific embodiment, Rₛ is optionally deuterated until fully deuterated.

m

In one embodiment, m is 0; In another embodiment, m is 1; In another embodiment, m is 2; in another embodiment, m is 3. In another embodiment, m is 4.

Any technical solution or any combination thereof in any of the above specific embodiments can be combined with any technical solution or any combination thereof in other specific embodiments. For example, any technical solution or any combination thereof of X₁ can be combined with any technical solution or any combination thereof of X₂, Y, R₁, R₂, R, L₁, L₂, Rₐ, R_{b}, R_{c}, R_{d}, L, R′, R″, R″, Rₓ, Rₛ, n, p, and m. The present invention is intended to include all combinations of these technical solutions, which are not listed one by one due to space limitations.

The present invention also provides a vector comprising a nucleotide sequence encoding the siRNA of the invention. The vector of the present invention can amplify or express a nucleotide encoding the siRNA of the invention linked thereto.

For example, a siRNA targeting the PCSK9 gene can be expressed from a transcription unit inserted into a DNA or RNA vector. Expression can be transient (within hours to weeks) or sustained (weeks to months or longer), depending on the particular construct used and the target tissue or cell type. A nucleotide encoding the siRNA can be introduced into a linear construct, a circular plasmid, or a viral vector. A nucleotide encoding the siRNA can be stably expressed by integration into the cell genome, or can be stably inherited and expressed extrachromosomally. Generally speaking, a vector expressing the siRNA is usually a DNA plasmid or a viral vector.

Viral vector systems comprising a sequence encoding the siRNA include, but are not limited to: (a) adenoviral vectors; (b) retroviral vectors; (c) adeno-associated viral vectors; (d) herpes simplex virus vectors; (e) SV40 vectors; (f) polyomavirus vectors; (g) papillomavirus vectors; (h) picornavirus vectors; (i) poxvirus vectors; and (j) helper virus-dependent or gutless adenoviral vectors.

The present invention also provides a cell comprising the siRNA or vector of the invention, wherein the siRNA or vector of the invention can be transcribed in the cell.

The present invention specifically relates to the following technical solutions:

1. A compound of formula (X), or a pharmaceutically acceptable salt, tautomer or stereoisomer thereof:

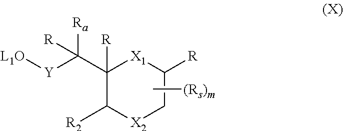

wherein, X₁ is a chemical bond, O or S; X₂ is a chemical bond or NR₁;
Y is a chemical bond or [C(Rₐ)(R_{b})]₁-4;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
R₂ is selected from H, D or OL₂;
R is selected from H, D, -L-OR′, -L-NR″R′″, R_{b} or —C(OL₂)(R_{c})(R_{d}), wherein R may be optionally deuterated or fully deuterated;
L₁ and L₂ are selected from H, a reactive phosphorus group or a protecting group;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a chemical bond or C₁₋₆ alkylene, wherein L may be optionally deuterated until fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated until fully deuterated;
n=0, 1, 2, 3, 4, 5, 6, 7, 8, 9 or 10;
p=0 or 1;
Rₛ is selected from H, D, C₁₋₆ alkyl or C₁₋₆ haloalkyl, wherein Rₛ may also be optionally deuterated or fully deuterated;
m=0, 1, 2, 3 or 4;
wherein an OL₂ group is present in either R or R₂.

2. A compound of formula (X) according to technical solution 1, or a pharmaceutically acceptable salt, tautomer, or stereoisomer thereof, wherein X₁ is a bond or O, preferably O.

3. A compound of formula (X) according to any one of technical solutions 1 or 2, or a pharmaceutically acceptable salt, tautomer, or stereoisomer thereof, wherein X² is a bond.

4. A compound of formula (X) according to any one of technical solutions 1 or 2, or a pharmaceutically acceptable salt, tautomer, or stereoisomer thereof, wherein X² is NR₁.

5. A compound of formula (X) according to any one of technical solutions 1 to 4, or a pharmaceutically acceptable salt, tautomer, or stereoisomer thereof, wherein Y is a bond.

6. A compound of formula (X) according to any one of technical solutions 1 to 4, or a pharmaceutically acceptable salt, tautomer, or stereoisomer thereof, wherein Y is [C(Rₐ)(R_{b})]₁₋₄, preferably C(Rₐ)(R_{b}).

7. A compound of formula (X) according to any one of technical solutions 1 to 6, or a pharmaceutically acceptable salt, tautomer, or stereoisomer thereof, wherein R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, or 5- to 14-membered heteroaryl, preferably from C₃₋₈ cycloalkyl, 3- to 10-membered heterocyclyl, C₀₋₁₀ aryl, or 5- to 14-membered heteroaryl, more preferably from C₃₋₈ cycloalkyl or 3- to 10-membered heterocyclyl, especially C₅₋₆ cycloalkyl.

8. A compound of formula (X) according to technical solution 7, or a pharmaceutically acceptable salt, tautomer, or stereoisomer thereof, wherein R₁ is optionally substituted with one or more groups selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, or C₁₋₆ haloalkyl, preferably with one or more groups selected from halogen, CN, OR′, SR′, NR″R′″, C₁₋₆ alkyl, or C₁₋₆ haloalkyl; wherein R₁ is also optionally deuterated or fully deuterated.

9. A compound of formula (X) according to any one of technical solutions 1 to 8, or a pharmaceutically acceptable salt, tautomer, or stereoisomer thereof, wherein R₂ is selected from H or D.

10. A compound of formula (X) according to any one of technical solutions 1 to 8, or a pharmaceutically acceptable salt, tautomer, or stereoisomer thereof, wherein R₂ is OL₂.

11. A compound of formula (X) according to any one of technical solutions 1 to 10, or a pharmaceutically acceptable salt, tautomer, or stereoisomer thereof, wherein R is selected from H, D, -L-OR′, -L-NR″R′″, or R_{b}, and is optionally deuterated or fully deuterated.

12. A compound of formula (X) according to any one of technical solutions 1 to 10, or a pharmaceutically acceptable salt, tautomer, or stereoisomer thereof, wherein one of R is —C(OL₂)(R_{c})(R_{d}), and is optionally deuterated or fully deuterated.

13. A compound of formula (X) according to any one of technical solutions 1 to 12, or a pharmaceutically acceptable salt, tautomer, or stereoisomer thereof, wherein La and L₂ are H.

14. A compound of formula (X) according to any one of technical solutions 1 to 12, or a pharmaceutically acceptable salt, tautomer, or stereoisomer thereof, wherein one of L₁ and L₂ is a reactive phosphorus group, preferably phosphoramidites (preferably —P(OCH₂CH₂CN)(N(iPr)₂)), H-phosphonates, alkyl-phosphonates, phosphates, or phosphate mimics, such as natural phosphates, borano thiophosphates, phosphorodithioates, boranyl phosphates, boranyl phosphorothioates, phosphonates, halogen-substituted phosphonates and phosphates, phosphoramidates, phosphodiesters, phosphotriesters, phosphorothioates, phosphotriesters, bisphosphates, and triphosphates.

15. A compound of formula (X) according to any one of technical solutions 1 to 12, or a pharmaceutically acceptable salt, tautomer, or stereoisomer thereof, wherein one of L₁ and L₂ is a protecting group, preferably a hydroxyl protecting group, such as trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), and p-methoxybenzyloxymethyl (PMBM).

16. A compound of formula (X) according to any one of technical solutions 1 to 15, or a pharmaceutically acceptable salt, tautomer, or stereoisomer thereof, wherein Rₐ, R_{b}, R_{c}, and R_{d} are independently selected from H, D, halogen, CN, —C₀₋₆ alkyl-OR′, —C₀₋₆ alkyl-NR″R′″, C₁₋₆ alkyl, C₁₋₆ haloalkyl, C₂₋₆ alkenyl, or C₂₋₆ alkynyl, preferably H, D, halogen, CN, C₁₋₆ alkyl, or C₁₋₆ haloalkyl; wherein Rₐ, R_{b}, R_{c}, and R_{d} are optionally deuterated or fully deuterated.

17. A compound of formula (X) according to any one of technical solutions 1 to 16, or a pharmaceutically acceptable salt, tautomer, or stereoisomer thereof, wherein the compound is selected from the following formulas:

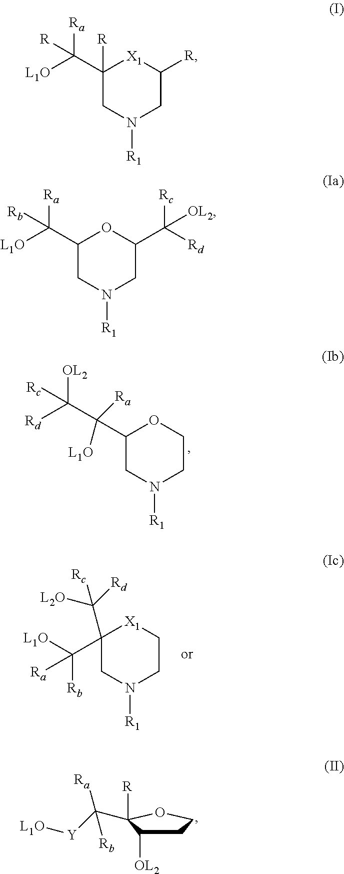

wherein each group is as defined in technical solutions 1 to 16.

18. A compound of formula (X) according to technical solution 17, or a pharmaceutically acceptable salt, tautomer, or stereoisomer thereof, which is a compound of formula (I):

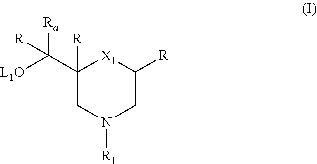

wherein,
X₁ is a bond, O or S;
R₁ is selected from C₁₋₂₀ alkyl, C₁₋₂₀ haloalkyl, C₂₋₂₀ alkenyl, C₂₋₂₀ alkynyl, C₃₋₂ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated until fully deuterated;
one of R is —C(OL₂)(R_{c})(R_{d}), and the other two are R_{b};
L₁ and L₂ are selected from H, a reactive phosphorus group or a protecting group;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4- to -L-7-membered heterocyclyl; wherein Rₐ, R_{b}, R. and Rₐ are optionally deuterated until fully deuterated;
wherein L is a chemical bond or C₁₋₆ alkylene, wherein L may be optionally deuterated until fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), wherein an aforementioned group is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated until fully deuterated;

n=0, 1, 2, 3, 4, 5, 6, 7, 8, 9 or 10;

p=0 or 1;

Preferably,
X₁ is a chemical bond, O or S;
R₁ is cyclohexyl, and is optionally deuterated until fully deuterated;
one of R is —C(OL₂)(R_{c})(R_{d}), and the other two are R_{b};
L₁ and L₂ are selected from DMTr or —P(OCH₂CH₂CN)(N(iPr)₂);
Rₐ, R_{b}, R_{c}, and R_{d} are independently selected from H or D.

19. A compound of formula (X) according to technical solution 17, or a pharmaceutically acceptable salt, tautomer, or stereoisomer thereof, wherein the compound is a compound of formula (Ia), (Ib), or (Ic):

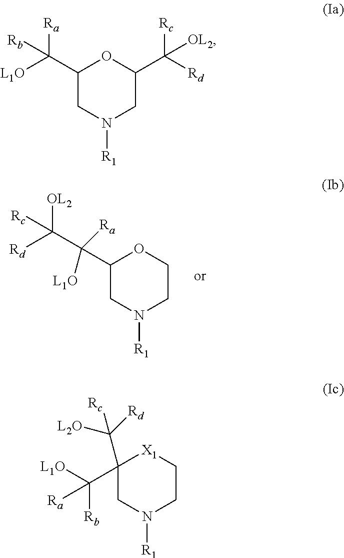

wherein,
X₁ is a chemical bond, O or S;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
L₁ and L₂ are selected from H, a reactive phosphorus group or a protecting group;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a bond or C₁₋₆ alkylene, wherein L may be optionally deuterated or fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated or fully deuterated;
n=0, 1, 2, 3, 4, 5, 6, 7, 8, 9 or 10;
p=0 or 1;
preferably,
X₁ is a bond, O or S;
R₁ is selected from C₃₋₆ cycloalkyl or 5- to 6-membered heterocyclyl, and is optionally deuterated until fully deuterated;
L₁ and L₂ are selected from DMTr or —P(OCH₂CH₂CN)(N(iPr)₂);
Rₐ, R_{b}, R_{c}, and R_{d} are independently selected from H, D, halogen, C₁₋₆ alkyl, or C₁₋₆ haloalkyl, and are optionally deuterated or fully deuterated;
preferably,
X₁ is a bond, O or S;
R₁ is C₄₋₆ cycloalkyl, and is optionally deuterated or fully deuterated;
L₁ and L₂ are selected from DMTr or —P(OCH₂CH₂CN)(N(iPr)₂);
Rₐ, R_{b}, R_{c}, and R_{d} are independently selected from H, D, halogen, C₁₋₄ alkyl, or C₁₋₄ haloalkyl, and are optionally deuterated or fully deuterated;
preferably,
X₁ is a bond or O;
R₁ is cyclohexyl, and is optionally deuterated until or deuterated;
L₁ and L₂ are selected from DMTr or —P(OCH₂CH₂CN)(N(iPr)₂);
Rₐ, R_{b}, R_{c}, and R_{d} are independently selected from H or D.

20. A compound of formula (X) according to technical solution 17, or a pharmaceutically acceptable salt, tautomer, or stereoisomer thereof, wherein the compound is one compound of formula (II):

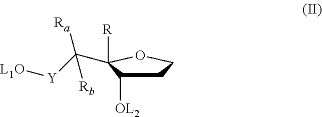

wherein,
Y is a bond or C(Rₐ)(R_{b});
R is selected from H, D, -L-OR′, or -L-NR″R′″, and is optionally deuterated or fully deuterated;
L₁ and L₂ are selected from H, a reactive phosphorus group or a protecting group;
Rₐ and R_{b} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4- to -L-7-membered heterocyclyl; wherein Rₐ and R_{b} are optionally deuterated or fully deuterated;
wherein L is a bond or C₁₋₆ alkylene, wherein L may be optionally deuterated or fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
preferably,
Y is a chemical bond or C(Rₐ)(R_{b});
R is selected from H, D, -L-OR′, or -L-NR″R′″, and is optionally deuterated until fully deuterated;
L₁ and L₂ are selected from DMTr or —P(OCH₂CH₂CN)(N(iPr)₂);
Rₐ and R_{b} are independently selected from H, D, halogen, -L-OR′, -L-NR″R′″, C₁₋₆ alkyl, C₁₋₆ haloalkyl, -L-C₃₋₈ cycloalkyl, or 4- to 7-membered heterocyclyl; wherein Rₐ and R_{b} are optionally deuterated until fully deuterated;
wherein L is a chemical bond or C₁₋₆ alkylene, wherein L may be optionally deuterated or fully deuterated;
R′, R″, and R″ are independently selected from H, C₁₋₆ alkyl, or C₁₋₆ haloalkyl; or R″ and R′″ together with the N atom connecting thereto form a 3- to 10-membered heterocyclyl;
preferably,
Y is a bond or C(Rₐ)(R_{b});
R is selected from H, D, —C₁₋₆ alkylene-NR″R′″, wherein R may be optionally deuterated or fully deuterated;
L₁ and L₂ are selected from DMTr or —P(OCH₂CH₂CN)(N(iPr)₂);
Rₐ and R_{b} are independently selected from H, D, halogen, C₁₋₄ alkyl, C₁₋₄ haloalkyl, —C₀₋₆ alkylene-NR″R′″, C₄₋₆ cycloalkyl, or 5- to 6-membered heterocyclyl; wherein Rₐ and R$ are optionally deuterated or fully deuterated;
wherein, R″ and R′″ are independently selected from H, C₁₋₄ alkyl, or C₁₋₄ haloalkyl; or R″ and R″ together with the N atom connecting thereto form a 5- to 6-membered heterocyclyl;
preferably,
Y is a bond or C(Rₐ)(R_{b});
R is selected from H, D, CH₂NMe₂, or CH₂-morpholinyl, and is optionally deuterated or fully deuterated;
L₁ and L₂ are selected from DMTr or —P(OCH₂CH₂CN)(N(iPr)₂);
Rₐ and R_{b} are independently selected from H, D, Me, NMe₂, morpholinyl, CH₂NMe₂, or CH₂— morpholinyl, and are optionally deuterated or fully deuterated.

21. A compound according to any one of technical solutions 1 to 20, or a pharmaceutically acceptable salt, tautomer or stereoisomer thereof, wherein the compound is selected from:

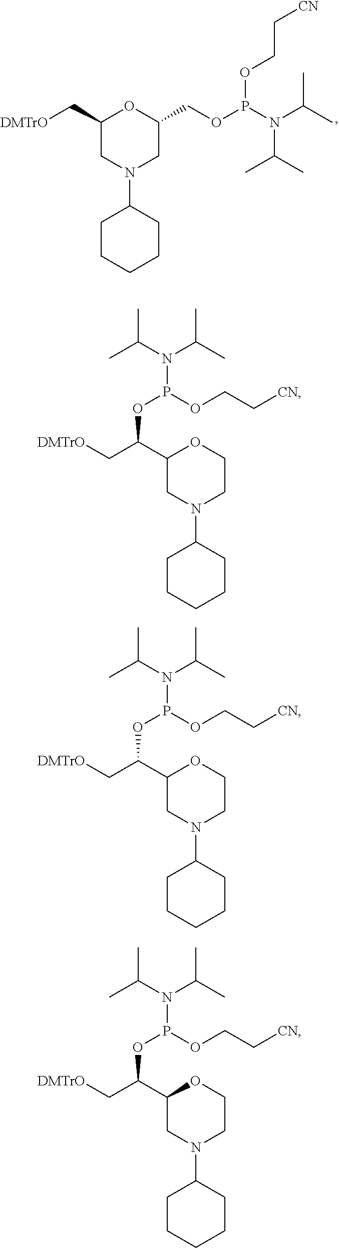

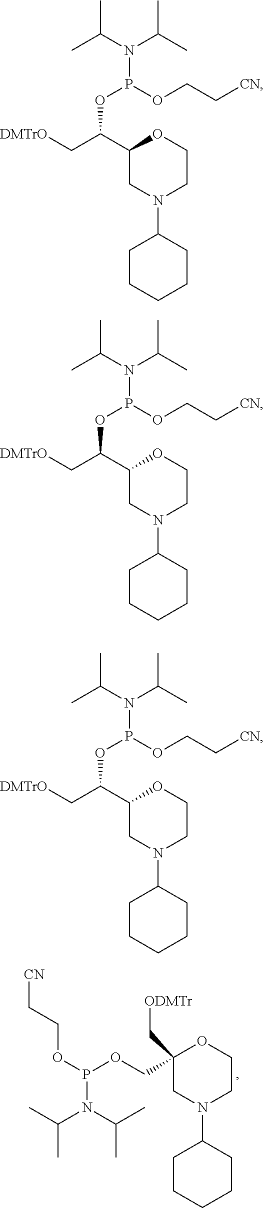

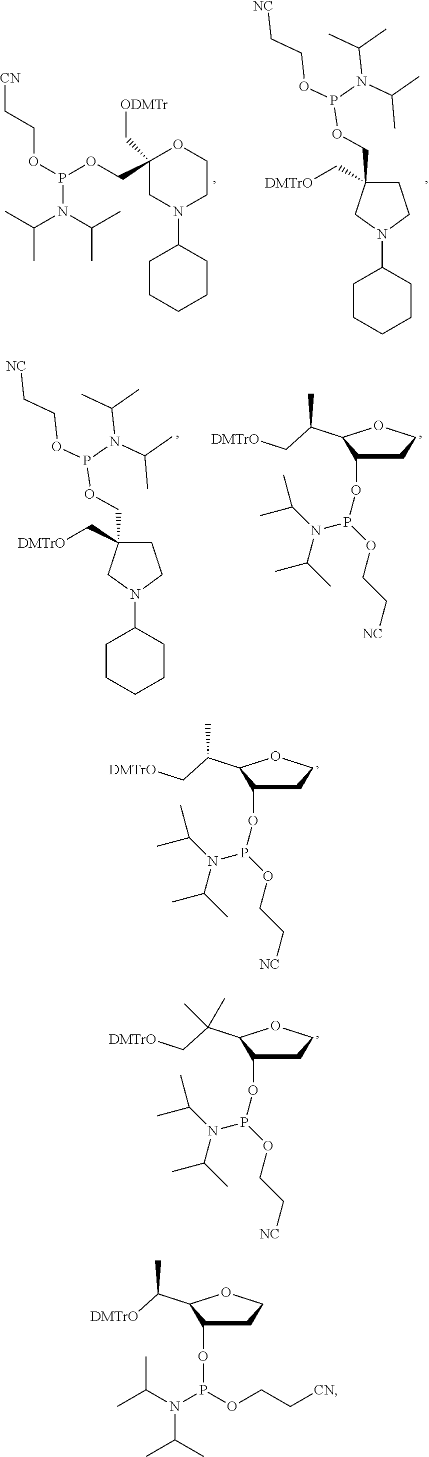

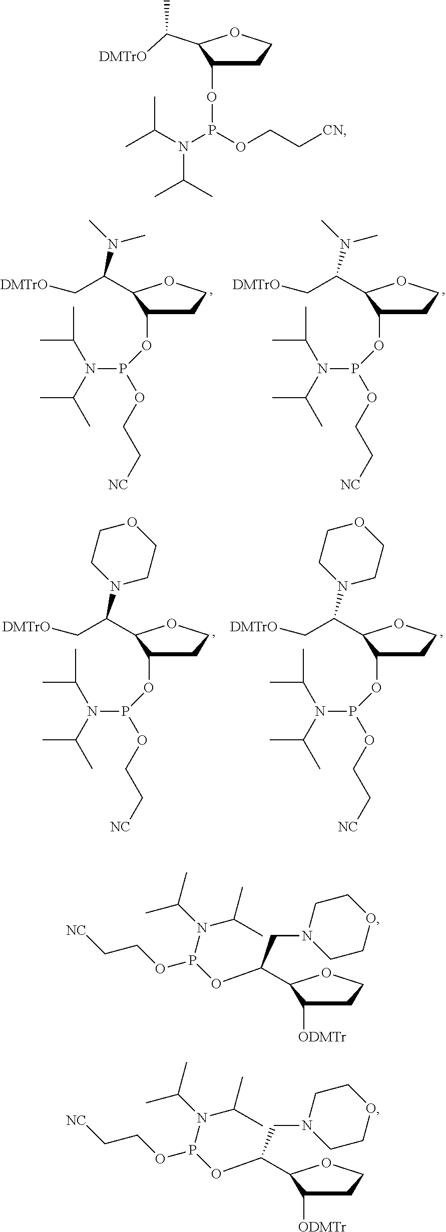

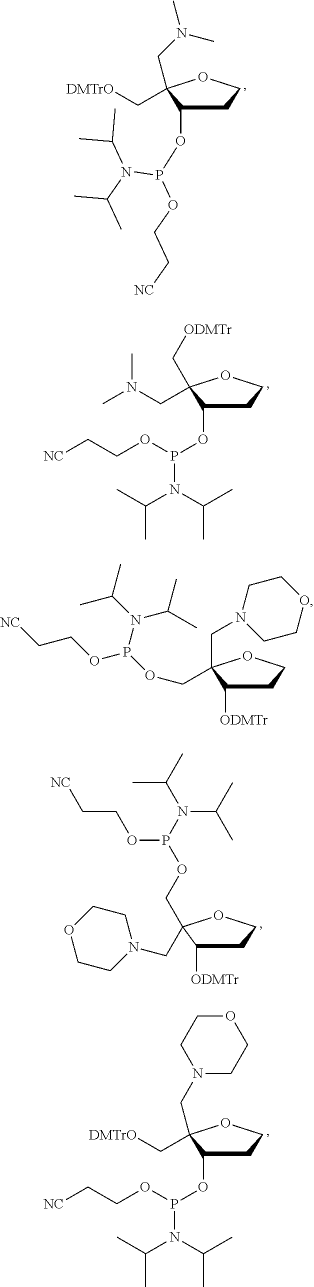

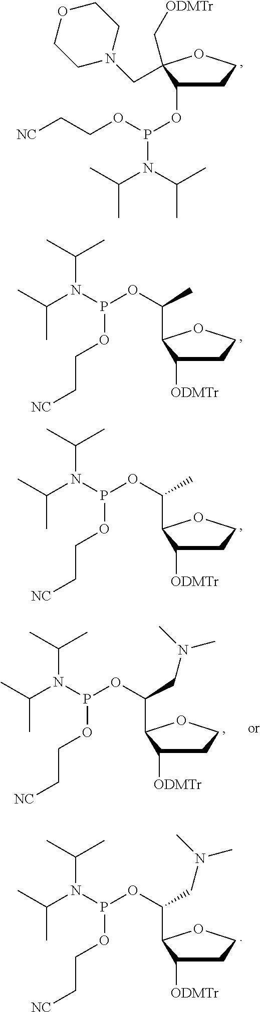

22. An oligonucleotide comprising at the 5′ and/or 3′ end one or more compounds of formula (X′):

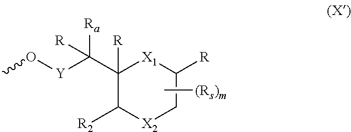

wherein,
R₂ is selected from H, D or O [symbol] ;
R is selected from H, D, -L-OR′, -L-NR″R′″, R_{b} or —C(O [symbol] )(R_{c})(R_{d}), wherein R may be optionally deuterated or fully deuterated;
provided that either of R and R₂ contains an O [symbol] group, wherein [symbol] represents a bond linked to a nucleotide of the oligonucleotide, and [symbol] at the terminal is linked to H, a reactive phosphorus group, or a protecting group; the other groups are as defined in any one of technical solutions 1 to 21.

23. An oligonucleotide according to technical solution 22, wherein the compound of formula (X′) is a compound of:

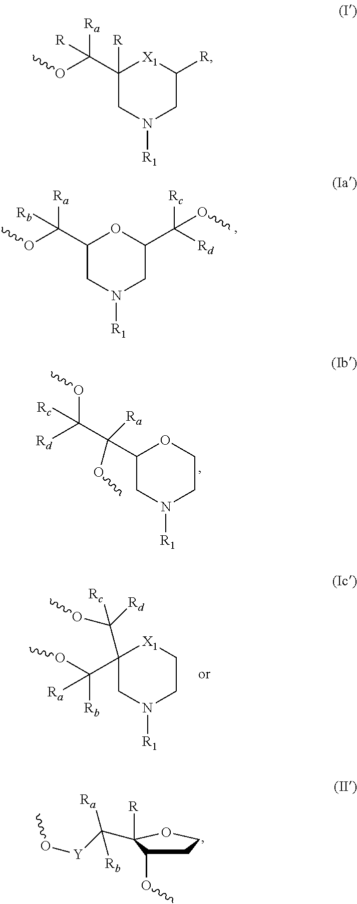

wherein each group is as defined in any one of technical solutions 1 to 22.

24 An oligonucleotide according to technical solution 22, wherein the compound of formula (X′) is a compound of:

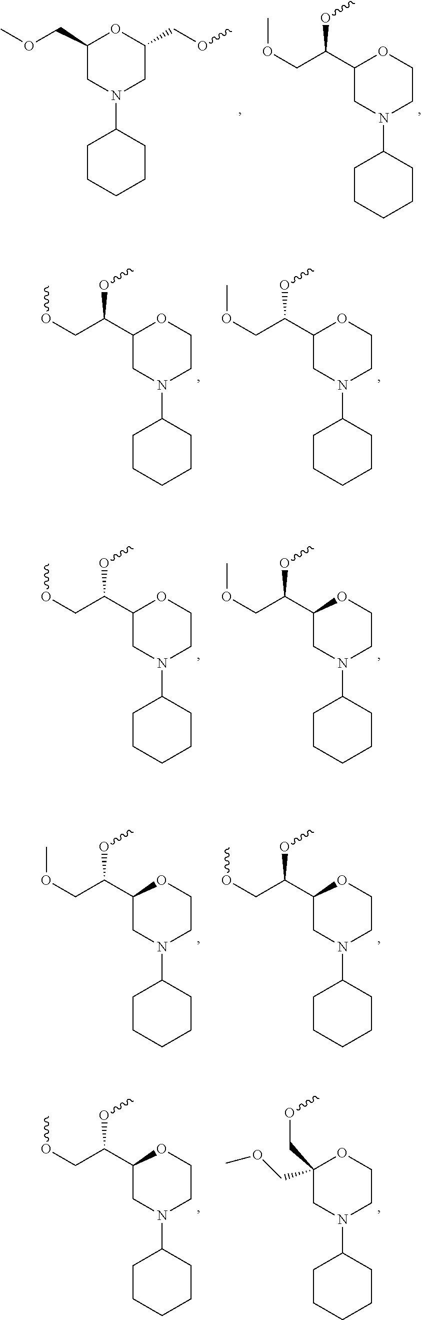

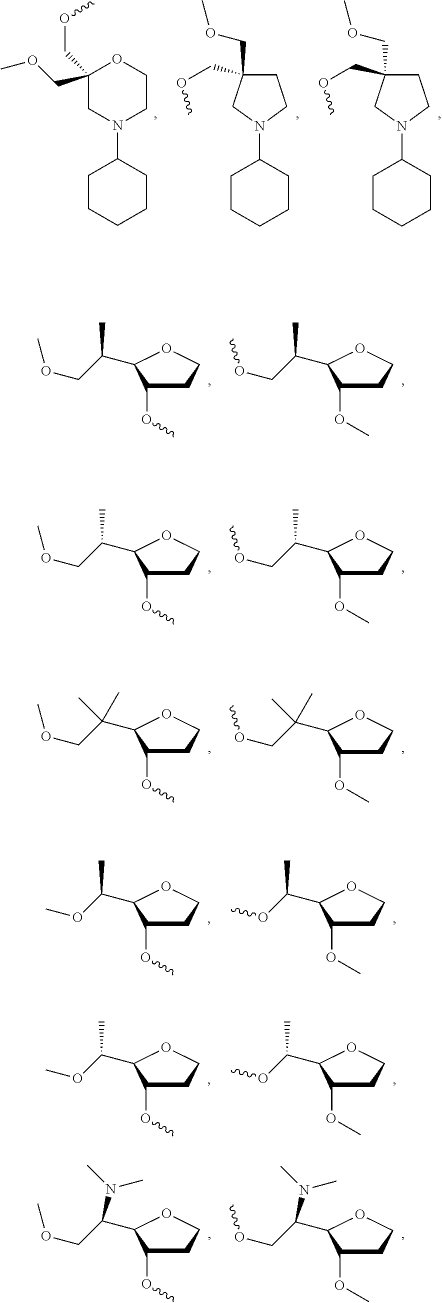

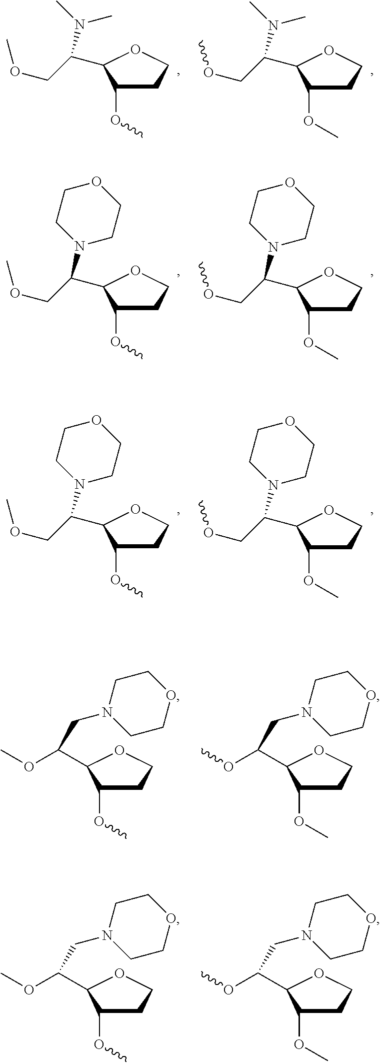

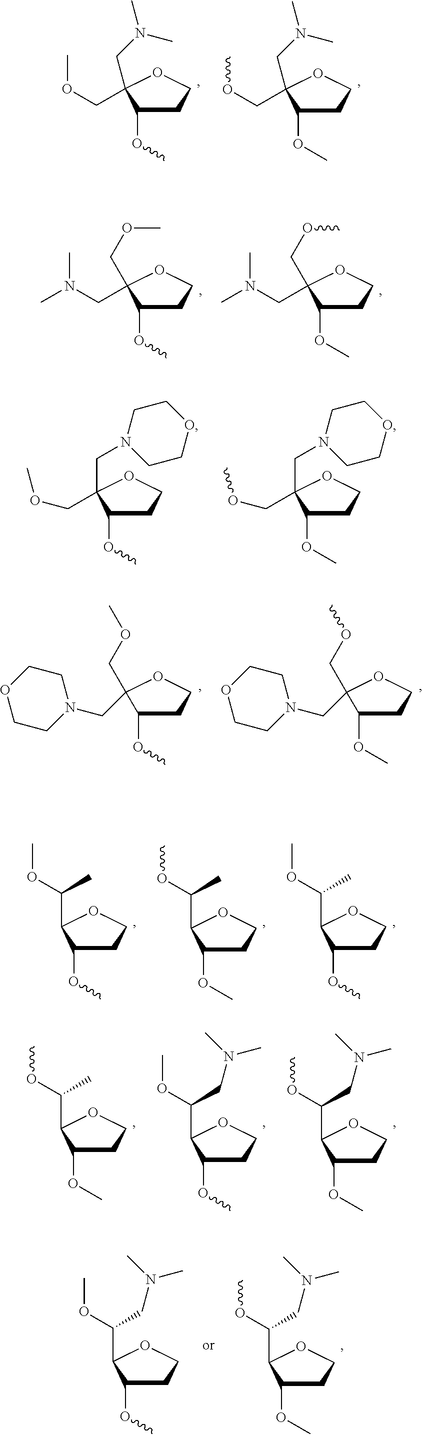

wherein the linkage is made from [symbol] to [symbol] of the compound in the 5′ to 3′ direction.

25. A dsRNA, wherein the dsRNA has a sense strand and an antisense strand between which the sequences are substantially complementary, wherein each strand has 14 to 30 nucleotides that are linked via a phosphate group, a phosphorothioate group, or other linker molecule, and wherein the said sense strand has the following structure:

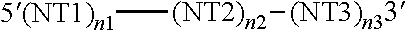

wherein,
NT1 and NT3 is independently a compound of formula (X′);
NT2 is a modified or unmodified nucleotide;
n1 is 0, 1, 2, or 3;
n2 is an integer in the range of 14 to 30;
n3 is 0, 1, 2, or 3;
and n1 and n3 are not both zero (0) simultaneously;

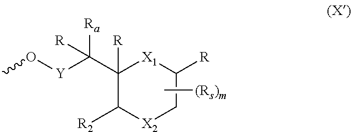

wherein,
R₂ is selected from H, D or O [symbol] ;
R is selected from H, D, -L-OR′, -L-NR″R′″, R_{b} or —C(O [symbol] )(R_{c})(R_{d}), wherein R may be optionally deuterated until fully deuterated;
X₁ is a chemical bond, O or S;
X₂ is a chemical bond or NR₁;
Y is a chemical bond or [C(Rₐ)(R_{b})]₁-4;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4- to -L-7-membered heterocyclyl; wherein Rₐ, R_{b}, R_{c} and R_{d} are optionally deuterated or fully deuterated;
wherein L is a chemical bond or C₁₋₆ alkylene, wherein L may be optionally deuterated or fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), ~C₀₋₆ alkylene-C₆₋₁₀ aryl or —Coo alkylene-(5- to 14-membered heteroaryl), wherein an aforementioned group is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated or fully deuterated;
n=0, 1, 2, 3, 4, 5, 6, 7, 8, 9 or 10;
p=0 or 1;
Rₛ is selected from H, D, C₁₋₆ alkyl or C₁₋₆ haloalkyl, wherein Rₛ may also be optionally deuterated or fully deuterated;
m=0, 1, 2, 3 or 4;
provided that one of R and R₂ contains an O [symbol] group, where [symbol] represents a bond linked to a nucleotide of the oligonucleotide, and [symbol] at the terminal is linked to H, a reactive phosphorus group, or a protecting group.

26. A dsRNA according to technical solution 25, wherein X₁ is a bond or O, preferably O.

27. A dsRNA according to technical solution 25 or 26, wherein X² is a bond.

28. A dsRNA according to technical solution 25 or 26, wherein X² is NR₁.

29. A dsRNA according to any one of technical solutions 25 to 28, wherein Y is a bond.

30. A dsRNA according to any one of technical solutions 25 to 28, wherein Y is [C(Rₐ)(R_{b})]₁₋₄, preferably C(Rₐ)(R_{b}).

31. A dsRNA according to any one of technical solutions 25 to 30, wherein R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, or 5- to 14-membered heteroaryl, preferably from C₃₋₈ cycloalkyl, 3- to 10-membered heterocyclyl, C₆₋₁₀ aryl, or 5- to 14-membered heteroaryl, more preferably from C₃₋₈ cycloalkyl, or 3- to 10-membered heterocyclyl, especially C₅₋₆ cycloalkyl.

32. A dsRNA according to technical solution 31, wherein R₁ is optionally substituted with one or more groups selected from halogen, CN, OR′, SR′, NR″R′″, C(O)OR′, C(S)ORₐₐ, C(O) NR″R′″, C(S) NR″R′″, OC(O)R′, OC(S)R′, NR″C(O)R′″, NR″C(S)R′″, C₁₋₆ alkyl, or C₁₋₆ haloalkyl, preferably with one or more groups selected from halogen, CN, OR′, SR′, NR″R′″, C₁₋₆ alkyl, or C₁₋₆ haloalkyl; wherein R₁ is also optionally deuterated or fully deuterated.

33. A dsRNA according to any one of technical solutions 25 to 32, wherein R₂ is selected from H or D.

34. A dsRNA according to any one of technical solutions 25 to 32, wherein R_{z} is O [symbol] .

35. A dsRNA according to any one of technical solutions 25 to 34, wherein R is selected from H, D, -L-OR′, -L-NR″R′″, or R_{b}, and is optionally deuterated or fully deuterated.

36. A dsRNA according to any one of technical solutions 25 to 34, wherein one of R is —C(O [symbol] )(R_{c})(R_{d}), and is optionally deuterated or fully deuterated.

37. A dsRNA according to any one of technical solutions 25 to 36, wherein Rₐ, R_{b}, R_{c}, and R_{d} are independently selected from H, D, halogen, CN, —C₀₋₆ alkyl-OR′, —C₀₋₆ alkyl-NR″R′″, C₁₋₆ alkyl, C₁₋₆ haloalkyl, C₂₋₆ alkenyl, or C₂₋₆ alkynyl, preferably from H, D, halogen, CN, C₁₋₆ alkyl, or C₁₋₆ haloalkyl; wherein Rₐ, R_{b}, R_{c}, and Rₐ are optionally deuterated or fully deuterated.

38. A dsRNA according to any one of technical solutions 25 to 37, wherein the compound of formula (X′) is selected from the following formulas:

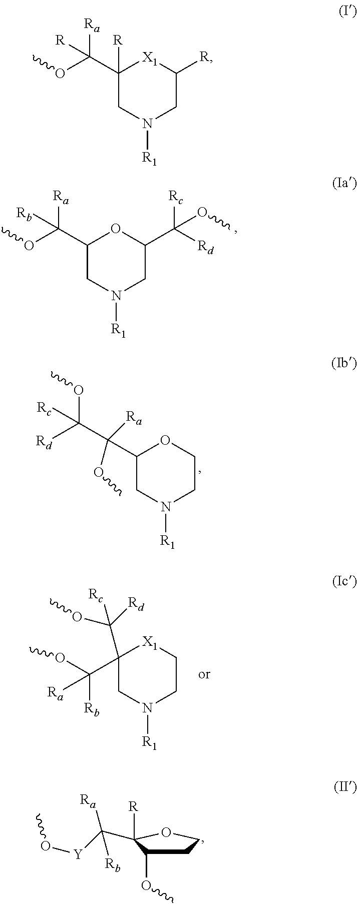

wherein each group is as defined in technical solutions 25 to 37.

39. A dsRNA according to technical solution 38, wherein the compound of formula (X′) is a compound of formula (I′):

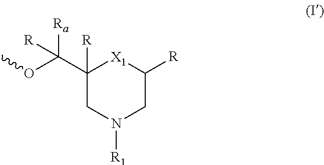

wherein,
X₁ is a chemical bond, O or S;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
one of R is —C(O [symbol] )(R_{c})(R_{d}), and the other two are R_{b},
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a bond or C₁₋₆ alkylene, wherein L may be optionally deuterated or fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated or fully deuterated;
n=0, 1, 2, 3, 4, 5, 6, 7, 8, 9 or 10;
p=0 or 1;
Preferably,
X₁ is a bond, O or S;
R₁ is cyclohexyl, and is optionally deuterated or fully deuterated;
one of R is —C(O [symbol] )(R_{c})(R_{d}), and the other two are R_{b};
Rₐ, R_{b}, R_{c}, and R_{d} are independently selected from H or D.

40. A dsRNA according to technical solution 38, wherein the compound of formula (X′) is a compound of formula (Ia′), (Ib′) or (Ic′):

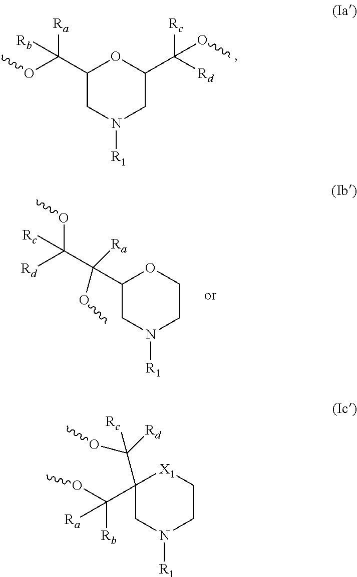

wherein,
X₁ is a chemical bond, O or S;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a bond or C₁₋₆ alkylene, wherein L may be optionally deuterated or fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated or fully deuterated;
n=0, 1, 2, 3, 4, 5, 6, 7, 8, 9 or 10;
p=0 or 1;
preferably,
X₁ is a bond, O or S;
R₁ is selected from C₃₋₆ cycloalkyl or 5- to 6-membered heterocyclyl, and is optionally deuterated or fully deuterated;
Rₐ, R_{b}, R_{c}, and R_{d} are independently selected from H, D, halogen, C₁₋₆ alkyl, or C₁₋₆ haloalkyl, and are optionally deuterated or fully deuterated;
preferably,
X₁ is a bond, O or S;
R₁ is C₄₋₆ cycloalkyl, and is optionally deuterated until fully deuterated;
Rₐ, R_{b}, R_{c}, and Rₐ are independently selected from H, D, halogen, C₁₋₄ alkyl, or C₁₋₄ haloalkyl, and are optionally deuterated or fully deuterated;
preferably,
X₁ is a bond or O;
R₁ is cyclohexyl, and is optionally deuterated or fully deuterated;
Rₐ, R_{b}, R_{c}, and R_{d} are independently selected from H or D.

41. A dsRNA according to technical solution 38, wherein the compound of formula (X′) is a compound of formula (II):

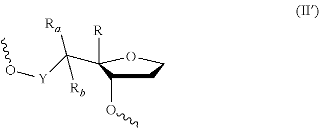

wherein,
Y is a chemical bond or C(Rₐ)(R_{b});
R is selected from H, D, -L-OR′, or -L-NR″R′″, and is optionally deuterated or fully deuterated;
Rₐ and R_{b} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4- to -L-7-membered heterocyclyl; wherein Rₐ and R_{b} are optionally deuterated or fully deuterated;
wherein L is a bond or C₁₋₆ alkylene, wherein L may be optionally deuterated until fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
preferably,
Y is a chemical bond or C(Rₐ)(R_{b});
R is selected from H, D, -L-OR′, or -L-NR″R′″, and is optionally deuterated or fully deuterated;
Rₐ and R_{b} are independently selected from H, D, halogen, -L-OR′, -L-NR″R′″, C₁₋₆ alkyl, C₁₋₆ haloalkyl, -L-C₃₋₈ cycloalkyl, or 4- to 7-membered heterocyclyl; wherein Rₐ and R_{b} are optionally deuterated or fully deuterated;
wherein L is a bond or C₁₋₆ alkylene, wherein L may be optionally deuterated or fully deuterated;
R′, R″, and R″ are independently selected from H, C₁₋₆ alkyl, or C₁₋₆ haloalkyl; or R″ and R′″ together with the N atom connecting thereto form a 3- to 10-membered heterocyclyl;
preferably,
Y is a bond or C(Rₐ)(R_{b});
R is selected from H, D, and —C₁₋₆ alkylene-NR″R′″, wherein R may be optionally deuterated or fully deuterated;
Rₐ and R_{b} are independently selected from H, D, halogen, C₁₋₄ alkyl, C₁₋₄ haloalkyl, —C₀₋₆ alkylene-NR″R′″, C₄₋₆ cycloalkyl, or 5- to 6-membered heterocyclyl; wherein Rₐ and R_{b} are optionally deuterated or fully deuterated;
wherein, R″ and R′″ are independently selected from H, C₁₋₄ alkyl, or C₁₋₄ haloalkyl; or R″ and R″ together with the N atom connecting thereto form a 5- to 6-membered heterocyclyl;
preferably,
Y is a chemical bond or C(Rₐ)(R_{b});
R is selected from H, D, CH₂NMe₂, or CH₂-morpholinyl, and is optionally deuterated or fully deuterated;
Rₐ and R_{b} are independently selected from H, D, Me, NMe₂, morpholinyl, CH₂NMe₂, or CH₂— morpholinyl, and are optionally deuterated or fully deuterated.

42. A dsRNA according to any one of technical solutions 25 to 41, wherein the compound of formula (X′) is a following compound:

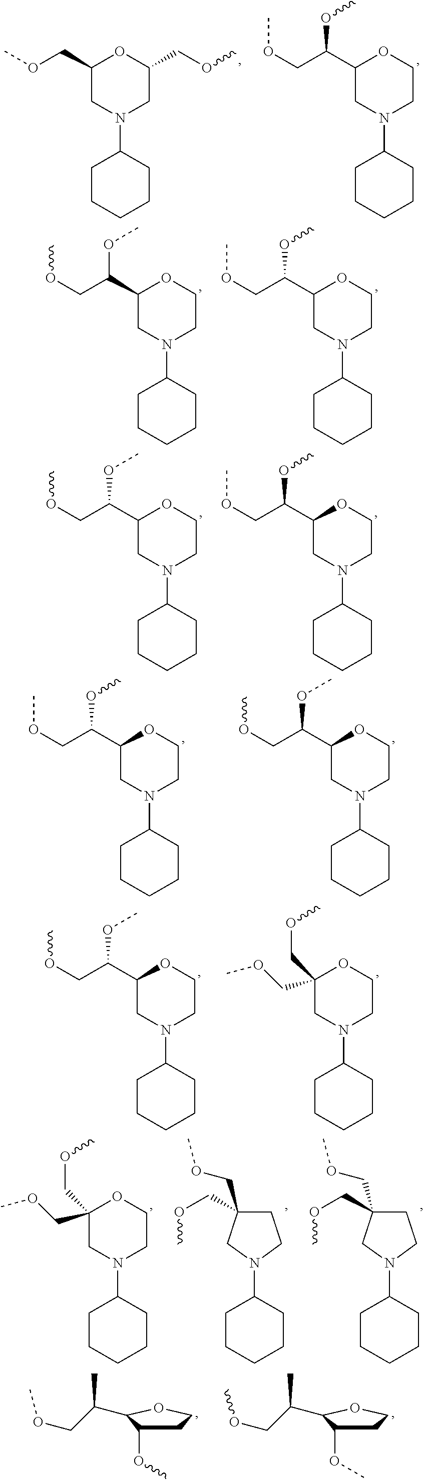

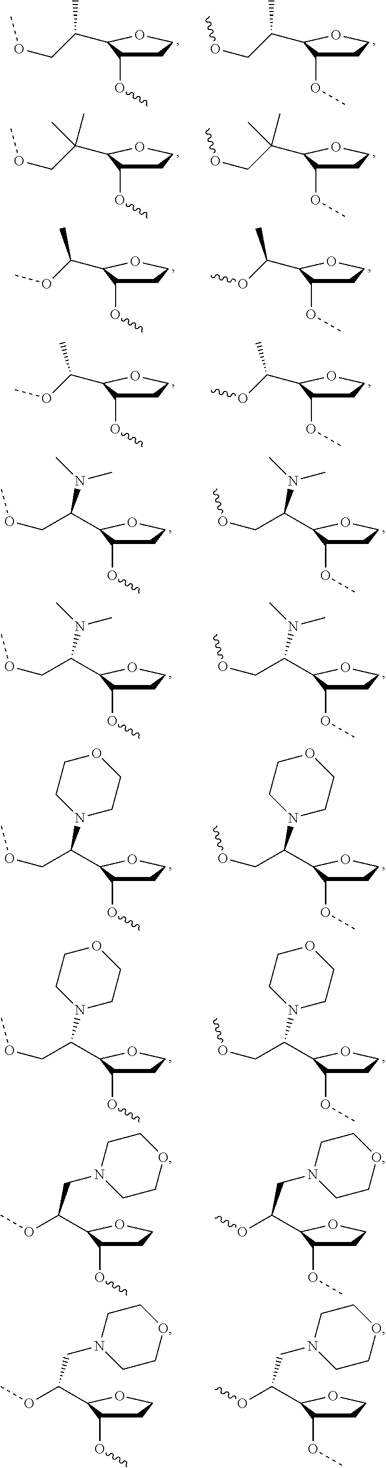

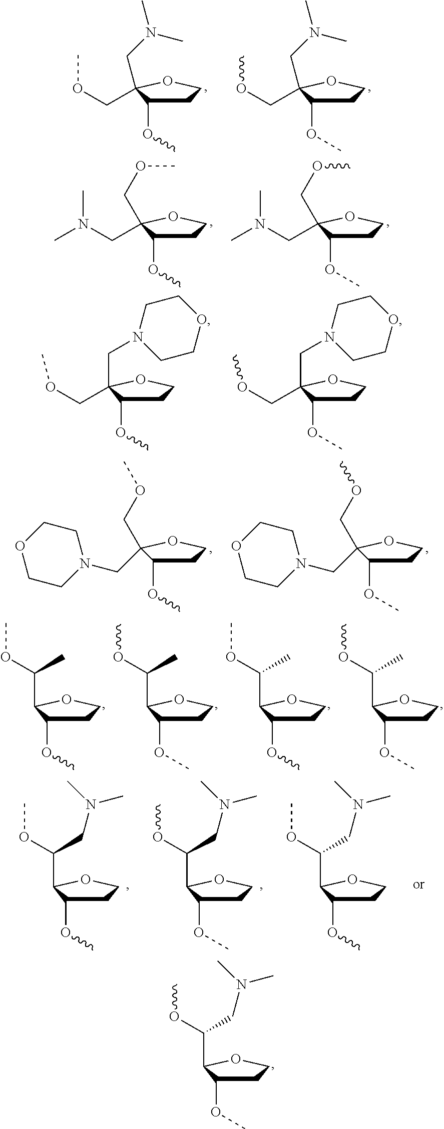

wherein the linkage is made from [symbol] to [symbol] of the compound in the 5′ to 3′ direction.

43. A dsRNA according to any one of technical solutions 25 to 42, wherein NT1, n1, NT3, and n3 are selected from the following table, and each nucleotide is preferably linked via a phosphate group or a phosphorothioate group:

| No. | NT1 | n1 | NT3 | n3 |
|---|---|---|---|---|
| 1 | N.A. | 0 | I′, Ia′, Ib′ or Ic′ | 1 |
| 2 | N.A. | 0 | I′, Ia′, Ib′ or Ic′ | 2 |
| 3 | N.A. | 0 | I′, Ia′, Ib′ or Ic′ | 3 |
| 4 | N.A. | 0 | II′ | 1 |
| 5 | N.A. | 0 | II′ | 2 |
| 6 | N.A. | 0 | II′ | 3 |
| 7 | I′, Ia′, Ib′ or Ic′ | 1 | N.A. | 0 |
| 8 | I′, Ia′, Ib′ or Ic′ | 2 | N.A. | 0 |
| 9 | I′, Ia′, Ib′ or Ic′ | 3 | N.A. | 0 |
| 10 | II′ | 1 | N.A. | 0 |
| 11 | II′ | 2 | N.A. | 0 |
| 12 | II′ | 3 | N.A. | 0 |
| 13 | I′, Ia′, Ib′ or Ic′ | 1 | I′, Ia′, Ib′ or Ic′ | 1 |
| 14 | I′, Ia′, Ib′ or Ic′ | 1 | I′, Ia′, Ib′ or Ic′ | 2 |
| 15 | I′, Ia′, Ib′ or Ic′ | 1 | I′, Ia′, Ib′ or Ic′ | 3 |
| 16 | I′, Ia′, Ib′ or Ic′ | 1 | II′ | 1 |
| 17 | I′, Ia′, Ib′ or Ic′ | 1 | II′ | 2 |
| 18 | I′, Ia′, Ib′ or Ic′ | 1 | II | 3 |
| 19 | I′, Ia′, Ib′ or Ic′ | 2 | I′, Ia′, Ib′ or Ic′ | 1 |
| 20 | I′, Ia′, Ib′ or Ic′ | 2 | I′, Ia′, Ib′ or Ic′ | 2 |
| 21 | I′, Ia′, Ib′ or Ic′ | 2 | I′, Ia′, Ib′ or Ic′ | 3 |
| 22 | I′, Ia′, Ib′ or Ic′ | 2 | II′ | 1 |
| 23 | I′, Ia′, Ib′ or Ic′ | 2 | II′ | 2 |
| 24 | I′, Ia′, Ib′ or Ic′ | 2 | II′ | 3 |
| 25 | I′, Ia′, Ib′ or Ic′ | 3 | I′, Ia′, Ib′ or Ic′ | 1 |
| 26 | I′, Ia′, Ib′ or Ic′ | 3 | I′, Ia′, Ib′ or Ic′ | 2 |
| 27 | I′, Ia′, Ib′ or Ic′ | 3 | I′, Ia′, Ib′ or Ic′ | 3 |
| 28 | I′, Ia′, Ib′ or Ic′ | 3 | II′ | 1 |
| 29 | I′, Ia′, Ib′ or Ic′ | 3 | II′ | 2 |
| 30 | I′, Ia′, Ib′ or Ic′ | 3 | II′ | 3 |
| 31 | II′ | 1 | I′, Ia′, Ib′ or Ic′ | 1 |
| 32 | II′ | 1 | I′, Ia′, Ib′ or Ic′ | 2 |
| 33 | II′ | 1 | I′, Ia′, Ib′ or Ic′ | 3 |
| 34 | II′ | 1 | II′ | 1 |
| 35 | II′ | 1 | II′ | 2 |
| 36 | II′ | 1 | II′ | 3 |
| 37 | II′ | 2 | I′, Ia′, Ib′ or Ic′ | 1 |
| 38 | II′ | 2 | I′, Ia′, Ib′ or Ic′ | 2 |
| 39 | II′ | 2 | I′, Ia′, Ib′ or Ic′ | 3 |
| 40 | II′ | 2 | II′ | 1 |
| 41 | II′ | 2 | II′ | 2 |
| 42 | II′ | 2 | II′ | 3 |
| 43 | II′ | 3 | I′, Ia′, Ib′ or Ic′ | 1 |
| 44 | II′ | 3 | I′, Ia′, Ib′ or Ic′ | 2 |
| 45 | II′ | 3 | I′, Ia′, Ib′ or Ic′ | 3 |
| 46 | II′ | 3 | II′ | 1 |
| 47 | II′ | 3 | II′ | 2 |
| 48 | II′ | 3 | II′ | 3 |

44. A dsRNA according to technical solution 25, wherein n1 is 0, NT3 is selected from compounds of formula (I), (Ia′), (Ib′), or (Ic′), and n3 is 1 or 2, preferably 2.

45. A dsRNA according to technical solution 25, wherein n1 is 1, NT1 is a compound of formula (II), and n3 is 0.

46. A dsRNA according to technical solution 25, wherein n1 is 0, NT3 is a compound of formula (II), and n3 is 1.

47. A dsRNA according to technical solution 25, wherein n1 is 1, NT1 is selected from a compound of formula (I′), (Ia′), (Ib′), or (Ic′), and n3 is 0.

48. A dsRNA according to technical solution 25, wherein n1 is 1, NT1 is a compound of formula (II′), NT3 is selected from a compound of formula (I′), (Ia′), (Ib′), or (Ic′), and n3 is 1 or 2, preferably 2.

49. A dsRNA according to technical solution 25, wherein n1 is 2, NT1 is a compound of formula (I), NT3 is selected from compounds of formula (I′), (Ia′), (Ib′), or (Ic′), and n3 is 1 or 2, preferably 2.

50. A dsRNA according to technical solution 25, wherein n1 is 2, NT1 is a compound of formula (I), (Ia′), (Ib′), or (Ic′), NT3 is selected from a compound of formula (I′), (Ia′), (Ib′), or (Ic′), and n3 is 2.

51. A dsRNA according to technical solution 25, wherein n1 is 1, NT1 is selected from a compound of formula (I′), (Ia′), (Ib′), or (Ic′), NT3 is selected from a compound of formula (I′), (Ia′), (Ib′), or (Ic′), and n3 is 1.

52. A dsRNA according to technical solution 25, wherein n1 is 1, NT1 is selected from a compound of formula (I′), (Ia′), (Ib′), or (Ic′), NT3 is selected from a compound of formula (I′), (Ia′), (Ib′), or (Ic′), and n3 is 2.

53. A dsRNA according to any one of technical solutions 25 to 52, wherein the dsRNA is further conjugated with a ligand moiety comprising N-acetylgalactosamine, preferably conjugated with the ligand moiety at the sense strand, preferably conjugated with the ligand moiety at the 3′ end of the sense strand, preferably conjugated with the ligand moiety at the 5′ end of the sense strand.

54. A dsRNA according to technical solution 53, wherein the ligand comprises one or more GalNAcs.

55. A dsRNA according to technical solution 54, wherein a ligand comprising GalNAc is conjugated to the 5′ end of the sense strand, and the said ligand is NAG37.

56. A dsRNA according to technical solution 54, wherein a ligand comprising GalNAc is conjugated to the 3′ end of the sense strand, and the said ligand is selected from L₉₆, GL6, or GL 12, preferably GL6.

57. A dsRNA according to technical solution 56, wherein n1 is 1, NT1 is a compound of formula (II′), NT3 is selected from a compound of formula (I′), (Ia′), (Ib′), or (Ic′), n3 is 2, and the 3′ end of the sense strand is conjugated to GL6.

58. A dsRNA according to technical solution 57, wherein n1 is 1, NT1 is a compound of formula (II), NT3 is selected from a compound of formula (I), (Ia′), (Ib′), or (Ic′), n3 is 2, and the 3′ end of the sense strand is conjugated to GL12.

59. A dsRNA according to any one of technical solutions 25 to 58, wherein the dsRNA is selected from small interfering RNA (siRNA) and short hairpin RNA (shRNA).

60. A dsRNA, wherein the dsRNA has a sense strand and an antisense strand between which the sequences are substantially complementary, wherein each strand has 14 to 30 nucleotides that are linked via a phosphate group, a phosphorothioate group, or other linker molecule, and wherein the said sense strand has one of the following modification patterns:

| | |
|---|---|
| MP1-1 | Ns-Ns-(N)n-STMs-STMs-LG |
| MP1-2 | Ns-Ns-(N)n-STM-STMs-LG |
| MP1-3 | Ns-(N)n-STMs-STMs-LG |
| MP1-4 | Ns-(N)n-STM-STMs-LG |
| MP1-5 | Ns-Ns-(N)n-STM-STM-LG |
| MP1-6 | (N)n-STMs-STMs-LG |
| MP1-7 | Ns-(N)n-STM-STM-LG |
| MP1-8 | (N)n-STM-STMs-LG |
| MP1 | (N)n-STM-STM-LG |
| MP2-1 | STMs-Ns-(N)n-Ns-STMs-LG |
| MP2-2 | STMs-Ns-(N)n-STMs-LG |
| MP2-3 | STMs-Ns-(N)n-STM-LG |
| MP2-4 | STMs-(N)n-Ns-STMs-LG |
| MP2-5 | STM-(N)n-Ns-STMs-LG |
| MP2-6 | STMs-(N)n-STMs-LG |
| MP2-7 | STMs-(N)n-STM-LG |
| MP2-8 | STM-(N)n-STMs-LG |
| MP2 | STM-(N)n-STM-LG |
| MP3-1 | STMs-Ns-(N)n-STMs-STMs-LG |
| MP3-2 | STMs-Ns-(N)n-STM-STMs-LG |
| MP3-3 | STMs-Ns-(N)n-STM-STM-LG |
| MP3-4 | STMs-(N)n-STMs-STMs-LG |
| MP3-5 | STM-(N)n-STMs-STMs-LG |
| MP3-6 | STMs-(N)n-STM-STMs-LG |
| MP3-7 | STMs-(N)n-STM-STM-LG |
| MP3-8 | STM-(N)n-STM-STMs-LG |
| MP3 | STM-(N)n-STM-STM-LG |
| MP4-1 | STMs-STMs-(N)n-STMs-STMs-LG |
| MP4-2 | STMs-STMs-(N)n-STM-STMs-LG |
| MP4-3 | STMs-STM-(N)n-STMs-STMs-LG |
| MP4-4 | STMs-STM-(N)n-STM-STMs-LG |
| MP4-5 | STM-STM-(N)n-STMs-STMs-LG |
| MP4-6 | STMs-STMs-(N)n-STM-STM-LG |
| MP4-7 | STM-STM-(N)n-STM-STMs-LG |
| MP4-8 | STMs-STM-(N)n-STM-STM-LG |
| MP4-9 | STM-STM-Ns-(N)n-Ns-N-STM-STMs-LG |
| MP4-10 | STM-STM-Ns-(N)n-STM-STM-LG |
| MP4 | STM-STM-(N)n-STM-STM-LG |
| MP5-1 | LGs-Ns-(N)n-Ns-STMs-STM |
| MP5-2 | LGs-(N)n-Ns-STMs-STM |
| MP5-3 | LGs-Ns-(N)n-STMs-STM |
| MP5-4 | LGs-(N)n-STMs-STM |
| MP5-5 | LGs-Ns-(N)n-STM-STM |
| MP5-6 | LG-(N)n-Ns-STMs-STM |
| MP5-7 | LGs-(N)n-STM-STM |
| MP5-8 | LG-(N)n-STMs-STM |
| MP5 | LG-(N)n-STM-STM |
| MP6-1 | LGs-STMs-(N)n-Ns-Ns-STM |
| MP6-2 | LGs-STMs-(N)n-Ns-STM |
| MP6-3 | LGs-STMs-(N)n-STM |
| MP6-4 | LGs-STM-(N)n-Ns-Ns-STM |
| MP6-5 | LG-STM-(N)n-Ns-Ns-STM |
| MP6-6 | LGs-STM-(N)n-Ns-STM |
| MP6-7 | LGs-STM-(N)n-STM |
| MP6-8 | LG-STM-(N)n-Ns-STM |
| MP6 | LG-STM-(N)n-STM |
| MP7-1 | LGs-STMs-(N)n-Ns-STMs-STM |
| MP7-2 | LGs-STMs-(N)n-STMs-STM |
| MP7-3 | LGs-STMs-(N)n-STM-STM |
| MP7-4 | LGs-STM-(N)n-Ns-STMs-STM |
| MP7-5 | LG-STM-(N)n-Ns-STMs-STM |
| MP7-6 | LGs-STM-(N)n-STMs-STM |
| MP7-7 | LGs-STM-(N)n-STM-STM |
| MP7-8 | LG-STM-(N)n-STMs-STM |
| MP7 | LG-STM-(N)n-STM-STM |
| MP8-1 | LGs-STMs-STM-(N)n-Ns-STMs-STM |
| MP8-2 | LGs-STM-STM-(N)n-Ns-STMs-STM |
| MP8-3 | LGs-STMs-STM-(N)n-STMs-STM |
| MP8-4 | LGs-STM-STM-(N)n-STMs-STM |
| MP8-5 | LGs-STMs-STM-(N)n-STM-STM |
| MP8-6 | LG-STM-STM-(N)n-Ns-STMs-STM |
| MP8-7 | LGs-STM-STM-(N)n-STM-STM |
| MP8-8 | LG-STM-STM-(N)n-STMs-STM |
| MP8 | LG-STM-STM-(N)n-STM-STM |

wherein N is a modified or unmodified nucleotide;
n is an integer in the range of 14 to 30;
STM is selected from a compound of formula (I), (Ia′), (Ib′), or (Ic′);
LG is a ligand comprising GalNAc, preferably GL6;
s is a phosphorothioate.

61. A dsRNA according to technical solution 60, wherein the modification pattern of the sense strand is selected from MP1-2, MP2-1, MP3-2, MP4-9, or MP4-10, preferably MP1-2 or MP2-1.

62. A dsRNA according to technical solution 61, wherein the modification pattern of the sense strand is MP1-2.

63. A dsRNA according to technical solution 61, wherein the modification pattern of the sense strand is MP2-1.

64. A dsRNA according to technical solution 61, wherein the modification pattern of the sense strand is MP3-2.

65. A dsRNA according to technical solution 61, wherein the modification pattern of the sense strand is MP4-9.

66. A dsRNA according to technical solution 61, wherein the modification pattern of the sense strand is MP4-10.

67. A dsRNA according to any one of technical solutions 61 to 66, wherein the STM is STM1, STM2, or STM3, preferably STM1.

68. A dsRNA according to any one of technical solutions 25 to 67, wherein the sense strand comprises one of the following nucleotide sequences:

FIN-FIN-FIN-CmsAmsGmUmGfUmUfCfUfUmGmCmUmCmUmAmUmAmAm-STM1-
STM1,
FIN-FIN-FIN-CmsAmsGmUmGfUmUfCfUfUmGmCmUmCmUmAmUmAmAm-
STM2aP1-STM2aP1,
FIN-FIN-FIN-
CmsAmsGmUmGfUmUfCfUfUmGmCmUmCmUmAmUmAmAm-STM2aP2-STM2aP2,
FIN-FIN-FIN-CmsAmsGmUmGfUmUfCfUfUmGmCmUmCmUmAmUmAmAm-STM3P1-
STM3P1,
FIN-FIN-FIN-CmsAmsGmUmGfUmUfCfUfUmGmCmUmCmUmAmUmAmAm-
STM3P2-STM3P2,
CmsAmsGmUmGfUmUfCfUfUmGmCmUmCmUmAmUmAmAm-STM1-
STM1s-GL6,
STM1s-AmsAmCmAmGmUmGfUmUfCfUfUmGmCmUmCmUmAmUmAmAms-STM1s-GL6,
STM1s-UmsCmCmAmCmGmUmUmGfCfUfUmGmAmAmAmUmUmGmAmAms-STM1s-
GL6,
STM1-STM1-
UmsCmCmAmCmGmUmUmGfCfUfUmGmAmAmAmUmUmGmAmsAm-STM1-STM1s-
GL6,
STM1s-UmsCmCmAmCmGmUmUmGfCfUfUmGmAmAmAmUmUmGmAmAm-
STM1-STM1s-GL6,
STM1-STM1-
UmsCmCmAmCmGmUmUmGfCfUfUmGmAmAmAmUmUmGmAmAm-STM1-STM1-GL6,
STM1s-AmsAmAmAmGmGmGmAmCfAfGfUmAmUmUmCmUmCmAmGmUms-STM1s-
GL6,
or
STM1-STM1-
AmsAmAmAmGmGmGmAmCfAfGfUmAmUmUmCmUmCmAmGmsUm-STM1-STM1s-
GL6.

69. A dsRNA according to technical solution 68, wherein the antisense strand comprises the following nucleotide sequence:

UmsUfsAmUmAmGfAmGmCmAmAmGmAmAfCmAfCmUmGmsUmsUm,
UmsUfsCmAfAmUfUmUfCmAfAmGfCmAfAmCfGmUfGmsGfsAm,
UmsAfsCmsUfGmAfUmCfAmAfAmUfAmUfGmUfUmGfAmGfsCm,
or
AmsCfsUmGfAmGfAmAfUmAfCmUfGmUfCmCfCmUfUmsUfsUm.

70. A vector comprising a nucleotide sequence encoding a dsRNA of any one of technical solutions 25 to 69.

71. A cell comprising a dsRNA of any one of technical solutions 25 to 69 or a vector of technical solution 70.

72. A pharmaceutical composition comprising a dsRNA of any one of technical solutions 25 to 69, a vector of technical solution 70, or a cell of technical solution 71, and optionally a pharmaceutically acceptable vector or excipient.

73. A kit comprising a dsRNA of any one of technical solutions 25 to 69, a vector of technical solution 70, or a cell of technical solution 71.

### EXAMPLES

The single-stranded sequences used in the examples are as follows:

Single strand No.: sequence (5′→3′)

SR004665S (SEQ ID NO. 1):
FIN-FIN-FIN-CmsAmsGmUmGfUmUfCfUfUmGmCmUmCmUmAmUmAmAm-STM1-STM1
SR004670S (SEQ ID NO. 2):
FIN-FIN-FIN-CmsAmsGmUmGfUmUfCfUfUmGmCmUmCmUmAmUmAmAm-STM2aP1-STM2aP1
SR004671S (SEQ ID NO. 3):
FIN-FIN-FIN-CmsAmsGmUmGfUmUfCfUfUmGmCmUmCmUmAmUmAmAm-STM2aP2-STM2aP2
SR004673S (SEQ ID NO. 4):
FIN-FIN-FIN-CmsAmsGmUmGfUmUfCfUfUmGmCmUmCmUmAmUmAmAmsdTsdT
SR004674S (SEQ ID NO. 5):
FIN-FIN-FIN-CmsAmsGmUmGfUmUfCfUfUmGmCmUmCmUmAmUmAmAm-IB
SR004675S (SEQ ID NO. 6):
FIN-FIN-FIN-CmsAmsGmUmGfUm UfCfUfUmGmCmUmCmUmAmUmAmAm
SR004668S (SEQ ID NO. 7):
FIN-FIN-FIN-CmsAmsGmUmGfUmUfCfUfUmGmCmUmCmUmAmUmAmAm-STM3P1-STM3P1
SR004669S (SEQ ID NO. 8):
FIN-FIN-FIN-CmsAmsGmUmGfUmUfCfUfUmGmCmUmCmUmAmUmAmAm-STM3P2-STM3P2
SR004441A (SEQ ID NO. 9):
UmsUfsAmUmAmGfAmGmCmAmAmGmAmAfCmAfCmUmGmsUmsUm
SR004469S (SEQ ID NO. 10):
CmsAmsGmUmGfUmUfCfUfUmGmCmUmCmUmAmUmAmsAms-GL6
SR011387S (SEQ ID NO. 11):
CmsAmsGmUmGfUmUfCfUfUmGmCmUmCmUmAmUmAmAm-STM1-STM1s-GL6
SR011388S (SEQ ID NO. 12):
STM1s-AmsAmCmAmGmUmGfUmUfCfUfUmGmCmUmCmUmAmUmAmAms-STM1s-GL6
SR011286S (SEQ ID NO. 13):
STM1s-UmsCmCmAmCmGmUmUmGfCfUfUmGmAmAmAmUmUmGmAmAms-STM1s-GL6
SR011542S (SEQ ID NO. 14):
STM1-STM1-UmsCmCmAmCmGmUmUmGfCfUfUmGmAmAmAmUmUmGmAmsAm-STM1-
STM1s-GL6
SR011543S (SEQ ID NO. 15):
STM1s-UmsCmCmAmCmGmUmUmGfCfUfUmGmAmAmAmUmUmGmAmAm-STM1-STM1s-GL6
SR002956S (SEQ ID NO. 16):
NAG37s-IBs-GmCmUmCmAmAmCmAmUfAfUfUmUmGmAmUmCmAmGmUmAms-IB
SR011283S (SEQ ID NO. 17):
CmsAmsCmGmUmUmGfCfUfUmGmAmAmAmUmUmGmAmsAms-GL6
SR011287S (SEQ ID NO. 18):
STM1-STM1-UmsCmCmAmCmGmUmUmGfCfUfUmGmAmAmAmUmUmGmAmAm-STM1-STM1-
GL6
SR011268A (SEQ ID NO. 19):
UmsUfsCmAfAmUfUmUfCmAfAmGfCmAfAmCfGmUfGmsGfsAm
SR002964A (SEQ ID NO. 20):
UmsAfsCmsUfGmAfUmCfAmAfAmUfAmUfGmUfUmGfAmGfsCm
SR011277S (SEQ ID NO. 21):
IBs-AmsAmAmAmGmGmGmAmCfAfGfUmAmUmUmCmUmCmAmGmUms-IBs-GL6
SR011278S (SEQ ID NO. 22):
STM1s-AmsAmAmAmGmGmGmAmCfAfGfUmAmUmUmCmUmCmAmGmUms-STM1s-GL6
SR011280S (SEQ ID NO. 23):
STM1-STM1-AmsAmAmAmGmGmGmAmCfAfGfUmAmUmUmCmUmCmAmGmsUm-STM1-
STM1s-GL6
SR011224A (SEQ ID NO. 24):
AmsCfsUmGfAmGfAmAfUmAfCmUfGmUfCmCfCmUfUmsUfsUm

The double-stranded sequences used in the examples are as follows:

| | Double Strand | Sense Strand | Antisense Strand |
|---|---|---|---|
| | No. | No. | No. |
| Example 5 | DR002346 | SR004665S | SR004441A |
| | DR002349 | SR004668S | SR004441A |
| | DR002350 | SR004669S | SR004441A |
| | DR002351 | SR004670S | SR004441A |
| | DR002352 | SR004671S | SR004441A |
| | DR002354 | SR004673S | SR004441A |
| | DR002355 | SR004674S | SR004441A |
| | DR002356 | SR004675S | SR004441A |
| Example 6 | DR002242 | SR004469S | SR004441A |
| | DR005781 | SR011387S | SR004441A |
| | DR005782 | SR011388S | SR004441A |
| Example 7 | DR005690 | SR011286S | SR011268A |
| | DR005921 | SR011542S | SR011268A |
| | DR005922 | SR011543S | SR011268A |
| | DR001479 | SR002956S | SR002964A |
| | DR005687 | SR011283S | SR011268A |
| | DR005691 | SR011287S | SR011268A |
| Example 8 | DR005681 | SR011277S | SR011224A |
| | DR005682 | SR011278S | SR011224A |
| | DR005684 | SR011280S | SR011224A |

The abbreviations used herein have the meaning as follows:
A, U, G, and C represent a natural adenine ribonucleotide, a uracil ribonucleotide, a guanine ribonucleotide, and a cytosine ribonucleotide, respectively.
T represents a thymine ribonucleotide.
d represents that the nucleotide adjacent to the right thereof is a deoxyribonucleotide. For example, dA, dT, dG, and dC represent an adenine deoxyribonucleotide, a thymine deoxyribonucleotide, a guanine deoxyribonucleotide, and a cytosine deoxyribonucleotide, respectively,
i represents an inosine ribonucleotide.
m represents that the nucleotide adjacent to the left thereof is a 2′-OCH₃ modified nucleotide. For example, Am, Um, Gm, and Cm represent 2′-OCH₃ modified A, U, G, and C, respectively.
f represents that the nucleotide adjacent to the left thereof is a 2′-F modified nucleotide. For example, Af, Uf, Gf, and Cf represent 2′-F modified A, U, G, and C, respectively.
“s” represents that the two nucleotides adjacent to the left and right thereof and/or the delivery moiety are linked by a phosphorothioate group.

VP represents that the nucleotide adjacent to the right thereof is a vinylphosphate modified nucleotide.

IB represents an inverted abasic deoxyribonucleotide, which can be of the following three structures depending on its position/linking method in siRNA.

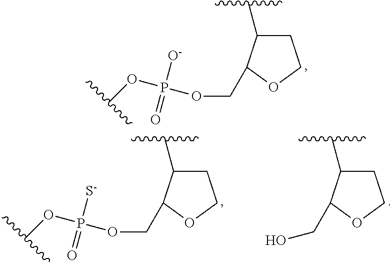 [SMILES: *OP(=O)([O-])OCC1OCCC1* ; *OP(=O)([S-])OCC1OCCC1* ; *C1CCOC1CO]

The compounds of the present invention are numbered and structured in oligonucleotides as follows, wherein the linkage is made from [symbol] to [symbol] of the compounds in the 5′ to 3′ direction.

Specifically, in the 5′ to 3′ direction, if the corresponding structure is located in the middle of the nucleic acid strand, [symbol] indicates that it is linked to the 3′ carbon or the corresponding position of the preceding nucleotide or nucleotide analog by a phosphate group, a phosphorothioate group, or other linking groups, and [symbol] indicates that it is linked to the 5′ carbon or the corresponding position of the next nucleotide or nucleotide analog by a phosphate group, a phosphorothioate group, or other linking groups, if the corresponding structure is located at a terminal of the nucleic acid strand, it may indicate linking to a hydrogen, a terminal modification, a terminal protecting group, a delivery moiety, or other structure available at a terminal of the nucleic acid strand; the optical isomers P1/P2 correspond to the corresponding P1/P2 in the examples:

| No. | Structural Formula | Optical Isomer No. |
|---|---|---|
| STM1 | [symbol] | None |
| STM2a and | [symbol] | STM2aP1 |
| STM2b | and | STM2aP2 |
| | | and |
| | | STM2bP1 |
| | | STM2bP2 |
| | 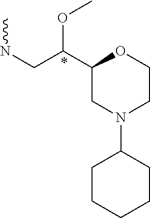 | |
| STM3 | 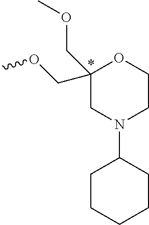 | STM3P1 STM3P2 |
| STM4 | 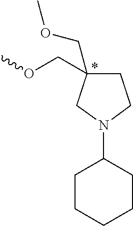 | STM4P1 STM4P2 |
| STM5a and STM5b | 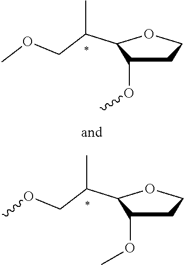 | STM5aP1 STM5aP2 STM5bP1 STM5bP2 |
| STM6a and STM6b | 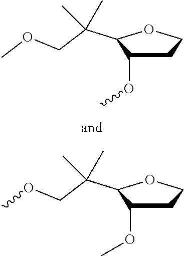 | None |
| STM7a and STM7b | 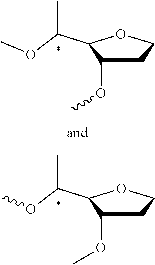 | STM7aP1 STM7aP2 and STM7bP1 STM7bP2 |
| STM8a and STM8b | 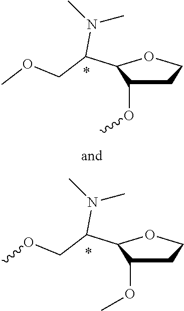 | STM8aP1 STM8aP2 and STM8bP1 STM8bP2 |
| STM9a and STM9b | 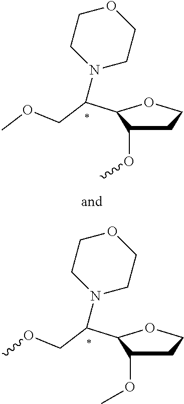 | STM9aP1 STM9aP2 and STM9bP1 STM9bP2 |
| STM10a and STM10b | 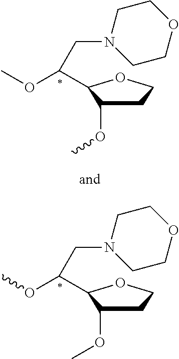 | STM10aP1 STM10aP2 and STM10bP1 STM10bP2 |
| STM11a and STM11b | 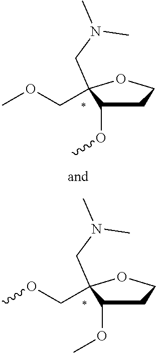 | STM11aP1 STM11aP2 and STM11bP1 STM11bP2 |
| STM12 | [symbol] | STM12P1 |
| | | STM12P2 |
| STM13 | [symbol] | STM13P1 |
| | | STM13P2 |
| STM14a and | [symbol] | STM14aP1 |
| STM14b | and | STM14aP2 |
| | | and |
| | | STM14bP1 |
| | | STM14bP2 |
| STM15a and | [symbol] | STM15aP1 |
| STM15b | and | STM15aP2 |
| | | and |
| | | STM15bP1 |
| | | STM15bP2 |

[symbol]

indicates data missing or illegible when filed

L₉₆ represents a GalNAc delivery moiety of the following structure well known in the art, wherein

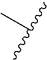

represents a position linked to siRNA by a phosphate group or a phosphorothioate group:

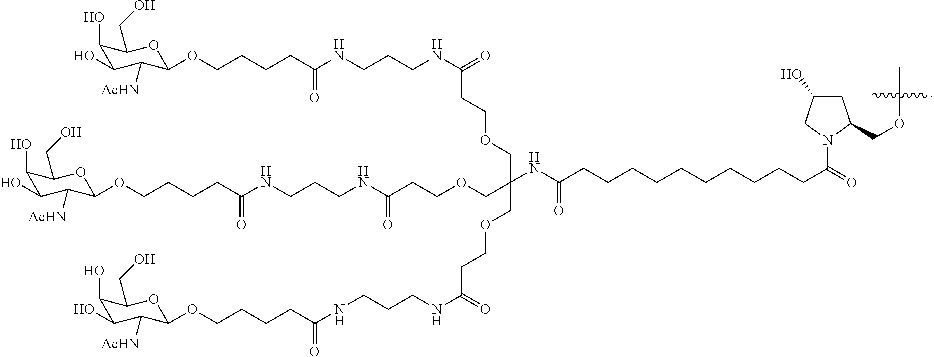 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

NAG37 represents a GalNAc delivery moiety of the following structure well known in the art, wherein

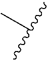

represents a position linked to siRNA:

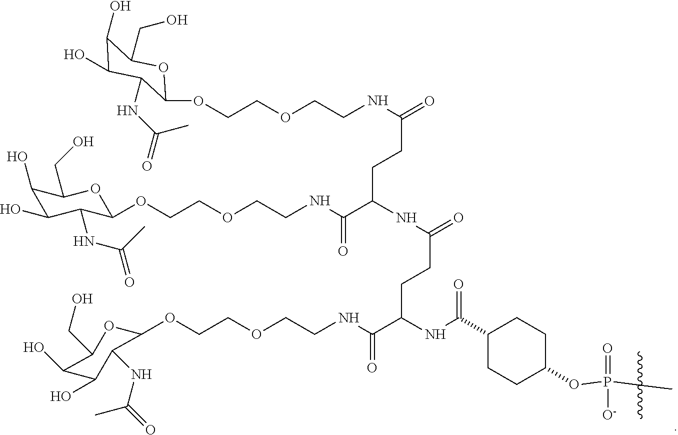

GL6 represents a GalNAc delivery moiety of the following structure, wherein

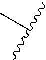

represents a position linked to siRNA by a phosphate group or a phosphorothioate group:

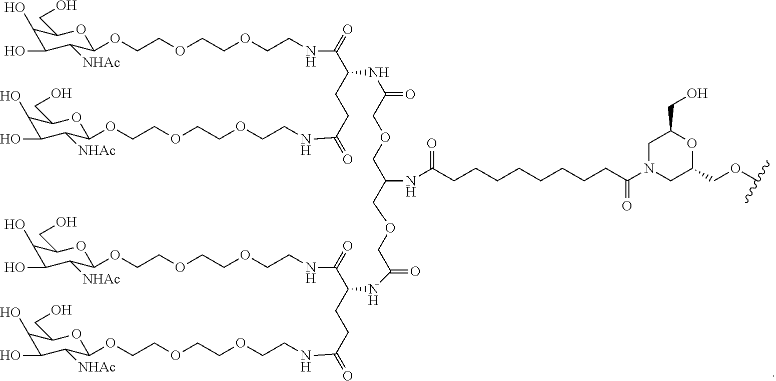 [SMILES: *OC[C@@H]1CN(C(=O)CCCCCCCCC(=O)NC(COCC(=O)N[C@H](CCC(=O)NCCOCCOCCOC2OC(CO)C(O)C(O)C2NC(C)=O)C(=O)NCCOCCOCCOC2OC(CO)C(O)C(O)C2NC(C)=O)COCC(=O)N[C@H](CCC(=O)NCCOCCOCCOC2OC(CO)C(O)C(O)C2NC(C)=O)C(=O)NCCOCCOCCOC2OC(CO)C(O)C(O)C2NC(C)=O)C[C@@H](CO)O1]

GL12 represents a GalNAc delivery moiety of the following structure, wherein

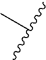

represents a position linked to siRNA by a phosphate group or a phosphorothioate group:

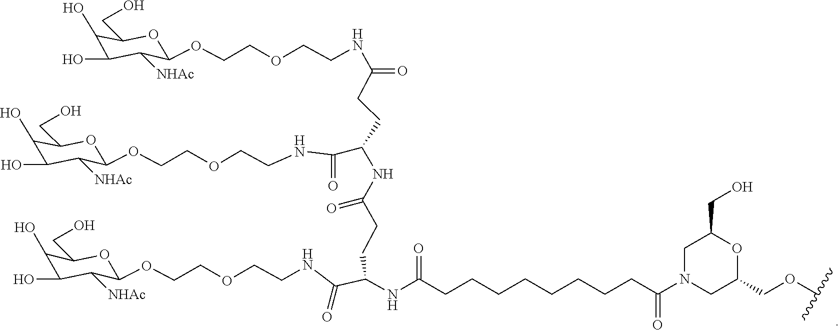 [SMILES: *OC[C@@H]1CN(C(=O)CCCCCCCCC(=O)N[C@@H](CCC(=O)N[C@@H](CCC(=O)NCCOCCOC2OC(CO)C(O)C(O)C2NC(C)=O)C(=O)NCCOCCOC2OC(CO)C(O)C(O)C2NC(C)=O)C(=O)NCCOCCOC2OC(CO)C(O)C(O)C2NC(C)=O)C[C@@H](CO)O1]

FIN-FIN-FIN-represents a GalNAc delivery moiety of the following structure, wherein

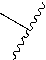

represents a position linked to siRNA:

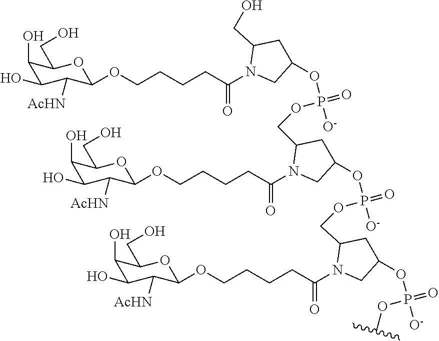 [SMILES: *OP(=O)([O-])OC1CC(COP(=O)([O-])OC2CC(COP(=O)([O-])OC3CC(CO)N(C(=O)CCCCOC4OC(CO)C(O)C(O)C4NC(C)=O)C3)N(C(=O)CCCCOC3OC(CO)C(O)C(O)C3NC(C)=O)C2)N(C(=O)CCCCOC2OC(CO)C(O)C(O)C2NC(C)=O)C1]

### Example 1: Synthesis of Compounds E1-P1 and E1-P2

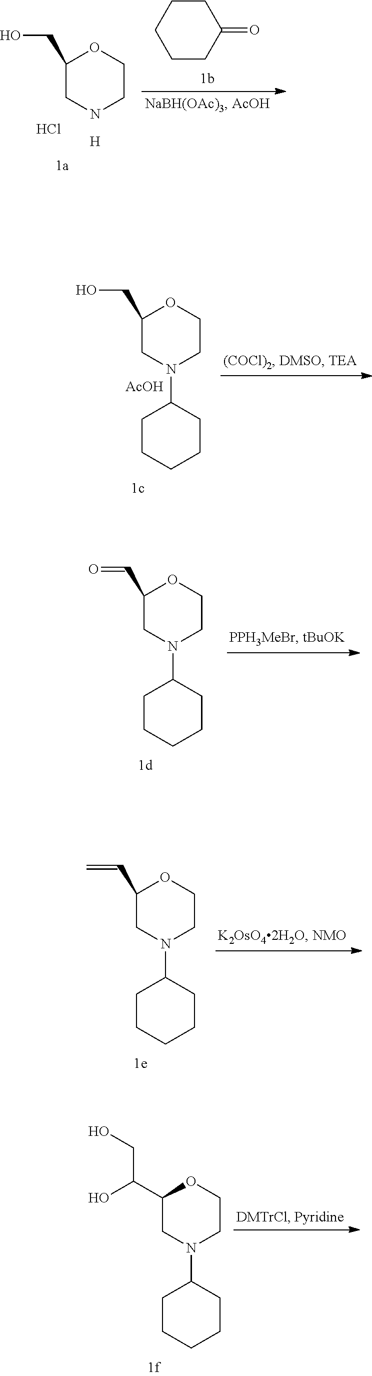

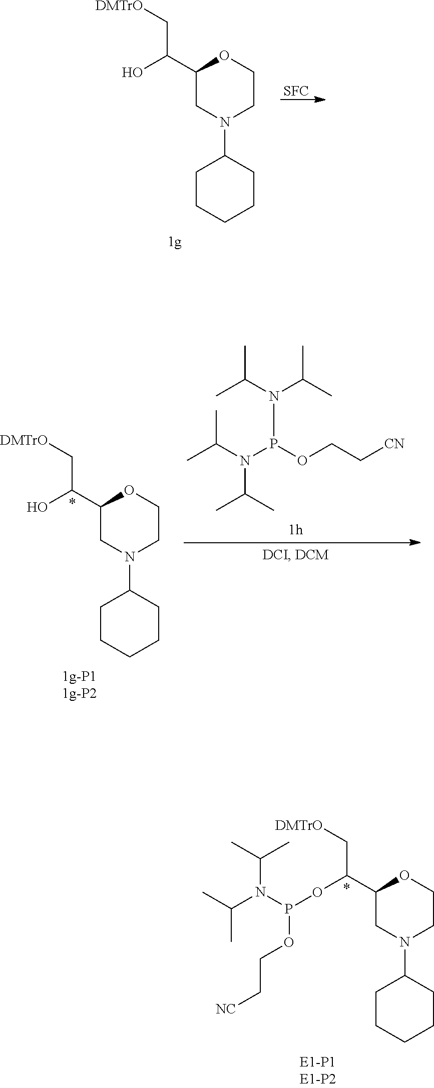

### 1. Preparation of Compound 1c

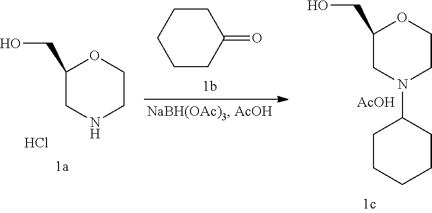 [SMILES: "AcOH" ; OC[C@@H]1CNCCO1 ; Cl ; O=C1CCCCC1 ; OC[C@@H]1CN(C2CCCCC2)CCO1]

To a mixture of compounds 1a (29.0 g, 188 mmol) and 1b (22.2 g, 226 mmol) in dichloromethane (580 mL), acetic acid (2.16 mL, 37.3 mmol) was added and stirred at room temperature for 3 h. Then sodium borohydride acetate (80.0 g, 377 mmol) was added, and the mixture was stirred at room temperature for an additional 12 h. After concentration under reduced pressure, the residue was separated by silica gel column chromatography (dichloromethane:methanol=10:1) to obtain the title compound 1c (58.9 g, yield 97.6%).

¹H NMR (400 MHz CDCl₃) δ 9.50 (brs, 3H), 4.07-3.94 (m, 3H), 3.64 (d, J=4.8 Hz, 2H), 3.39-3.36 (m, 2H), 3.30-3.27 (m, 2H), 2.88 (tt, J₁=11.6 Hz, J₂=3.2 Hz, 1H), 2.74 (td, J₁=11.6 Hz, J₂=4.8 Hz, 1H), 2.64 (t, J=11.2 Hz, 1H), 2.08-2.05 (m, 2H), 2.01 (s, 6.54H), 1.89-1.86 (m, 2H), 1.69-1.65 (m, 1H), 1.44-1.23 (m, 4H), 1.17-1.06 (m, 1H).

### 2. Preparation of Compound 1d

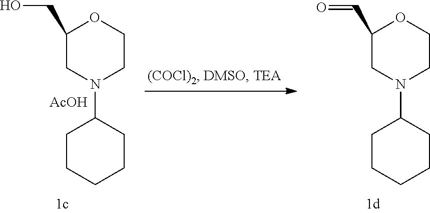 [SMILES: "AcOH" ; OC[C@@H]1CN(C2CCCCC2)CCO1 ; O=C[C@@H]1CN(C2CCCCC2)CCO1]

Oxalyl chloride (4.37 g, 34.4 mmol) was dissolved in dichloromethane (100 mL), and vacuum and nitrogen were applied. After cooling to −65° C., dimethyl sulfoxide (2.69 g, 34.4 mmol) was added dropwise, and the reaction mass was stirred for 15 min. Then compound 1c (5.0 g, 15.6 mmol) and triethylamine (16.3 mL, 117 mmol) were added, and stirring was continued for an additional 1 h. The reaction was quenched with water. After the reaction mass reached room temperature, ethyl acetate (200 mL×2) was added for extraction. The organic phases were combined and washed with saturated sodium chloride solution, dried with anhydrous sodium sulfate, and filtered. The organic solvent was concentrated under reduced pressure to obtain the title compound 1d (12.0 g), which was used directly in the next step.

¹H NMR (400 MHz CDCl₃) δ 9.65 (s, 1H), 4.02-3.96 (m, 2H), 3.75-3.69 (m, 3H), 2.97-2.94 (m, 1H), 2.70-2.67 (m, 1H), 2.46-2.40 (m, 1H), 2.37-2.31 (1H), 2.24-2.22 (m, 1H), 1.85-1.78 (m, 4H), 1.63-1.60 (m, 1H), 1.28-1.05 (m, 5H).

### 3. Preparation of Compound 1e

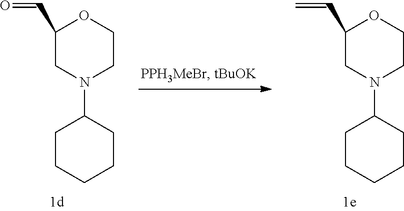

Triphenylmethylphosphine bromide (16.3 g, 45.6 mmol) was suspended in tetrahydrofuran (150 mL), and vacuum and nitrogen were applied. Potassium tert-butoxide (45.6 mL, 1 M in tetrahydrofuran) was added, and the mixture was stirred at room temperature for 1 h. Compound 1d (3.00 g, 15.2 mmol) was dissolved in tetrahydrofuran (10 mL) and then added dropwise to the aforementioned mixture, and the reaction mass was stirred at room temperature for 12 h. The reaction mass was poured into a saturated ammonium chloride solution, adjusted to pH 8 with saturated sodium bicarbonate, and extracted with ethyl acetate (100 mL×2). The organic phases were combined and washed with saturated sodium chloride solution, dried with anhydrous sodium sulfate, and filtered. The organic solvent was concentrated under reduced pressure, and the residue was separated by silica gel column chromatography (petroleum ether:ethyl acetate=10:1) to obtain the title compound 1e (10.1 g, yield 59.5%).

¹H NMR (400 MHz CDCl₃) δ 5-85-5.77 (m, 1H), 5.33-5.28 (m, 1H), 5.17-5.15 (m, 1H), 4.04-3.97 (m, 1H), 3.94-3.91 (m, 1H), 3.72-3.67 (m, 1H), 2.82-2.79 (m, 1H), 2.72-2.70 (m, 1H), 2.39-2.33 (m, 1H), 2.26-2.17 (m, 1H), 2.13-2.07 (m, 1H), 1.89-1.87 (m, 2H), 1.81-1.78 (m, 2H), 1.64-1.62 (m, 1H), 1.26-1.09 (m, 5H).

### 4. Preparation of Compound 1f

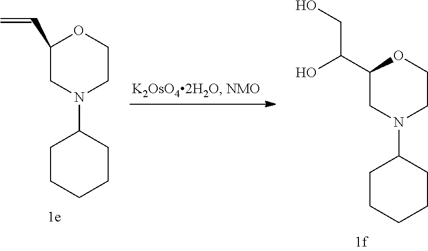 [SMILES: C=C[C@@H]1CN(C2CCCCC2)CCO1 ; OCC(O)[C@@H]1CN(C2CCCCC2)CCO1]

To a mixture of compound 1e (6.00 g, 21.5 mmol) and N-methylmorpholine oxide (3.28 g, 27.9 mmol) in acetone, an aqueous solution of K₂OsO₄·2H₂O (158 mg, 430 μmol) was added, and the reaction mass was stirred at room temperature for 12 h. The reaction mass was filtered. The filtrate was concentrated under reduced pressure, and the residue was separated by silica gel column chromatography (dichloromethane:methanol=10:1) to obtain the title compound 1f (9.01 g, yield 82.1%).

¹H NMR (400 MHz CD₃OD) δ 3.91-3.84 (m, 1H), 3.69-3.44 (m, 5H), 3.10-2.74 (m, 2H), 2.38-2.11 (m, 3H), 1.96-1.94 (m, 2H), 1.84-1.81 (m, 2H), 1.67-1.64 (m, 1H), 1.34-1.10 (m, 5H),

### 5. Preparation of Compound 1g

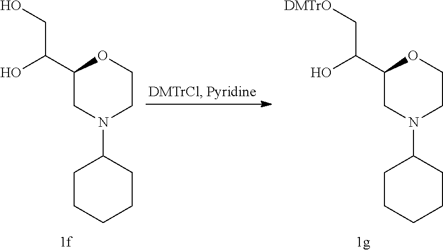

To a solution of compound 1f (2.01 g, 7.85 mmol) in pyridine (12 mL), DMTrCl (2.79 g, 8.24 mmol) was added, and the reaction mass was stirred at room temperature for 1 h. The reaction was quenched with methanol. After multiple batches were combined and concentrated under reduced pressure, the residue was separated by silica gel column chromatography (dichloromethane:methanol=10:1) to obtain the title compound 1g (4.01 g, yield 77.5%).

¹H NMR (400 MHz DMSO-d₆) δ 7.42-7.39 (m, 2H), 7.31-7.18 (m, 7H), 6.89-6.86 (m, 4H), 4.92-4.73 (m, 1H), 3.77-3.67 (m, 7H), 3.56-3.33 (m, 3H), 3.08-2.55 (m, 4H), 2.21-1.97 (m, 3H), 1.78-1.54 (m, 5H), 1.23-1.02 (m, 5H).

6. Preparation of Compounds 1g-P1 and 1g-P2

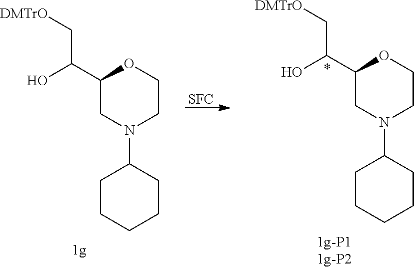

Compound 1g (6.02 g, 11.2 mmol) was separated by SFC using a chiral column (column: DAICEL CHIRALPAK IG (250 mm*50 mm, 10 μm); mobile phase: A: CO₂, B: [0.1% NH₃·H₂O in IPA]; B %: 60%-60%, 13 min) to obtain compounds 1g-P1 (3.20 g) and 1g-P2 (1.80 g).

Compound 1g-P1:

m/z: ES+ [M+H]+532.2

HPLC: retention time 1.580 min (column: Chiralpak IG-3, 50×4.6 mm I.D., 3 μm; mobile phase: A: CO₂, B: IPA (0.1% IPA, v/v); gradient: 0-0.2 min 5% B, 0.2-1.2 min 5-50% B, 1.2-2.2 min 50% B, 2.2-2.6 min 50%-5% B, 2.6-3.0 min 5% B; flow rate: 3.4 mL/min; column temperature: 35° C.)

¹H NMR (400 MHz DMSO-d₆) δ 7.42-7.40 (m, 2H), 7.31-7.12 (m, 7H), 6.88-6.86 (m, 4H), 4.94 (d, J=6.0 Hz, 1H), 3.73 (s, 6H), 3.69-3.67 (m, 1H), 3.56-3.50 (m, 1H), 3.39-3.37 (m, 1H), 3.01-2.95 (m, 2H), 2.87-2.84 (m, 1H), 2.58-2.55 (m, 1H), 2.20-2.15 (m, 2H), 2.02-1.97 (m, 1H), 1.78-1.54 (m, 5H), 1.23-1.01 (m, 5H).

Compound 1g-P2:

m/z: ES+ [M+H]+532.2

HPLC: retention time 2.048 min (column: Chiralpak IG-3, 50×4.6 mm I.D., 3 μm; mobile phase: A: CO₂, B: IPA (0.1% IPA, v/v); gradient: 0-0.2 min 5% B, 0.2-1.2 min 5-50% B, 1.2-2.2 min 50% B, 2.2-2.6 min 50%-5% B, 2.6-3.0 min 5% B; flow rate: 3.4 mL/min; column temperature: 35° C.)

¹H NMR (400 MHz DMSO-d₆) δ 7.41-7.39 (m, 2H), 7.31-7.15 (m, 7H), 6.88-6.86 (m, 4H), 4.75 (d, J=4.4 Hz, 1H), 3.85-3.65 (m, 7H), 3.54-3.41 (m, 3H), 3.08-3.05 (m, 1H), 2.85-2.82 (m, 1H), 2.59-2.57 (m, 2H), 2.17-2.07 (m, 3H), 1.71-1.53 (m, 4H), 1.24-1.02 (m, 5H).

### 7. Preparation of Compound E1-P1

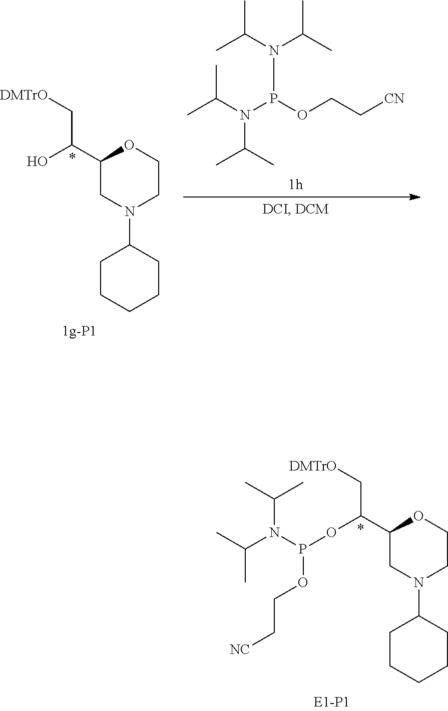

To a solution of compound 1g-P1 (1.50 g, 2.82 mmol) in dichloromethane (15 mL), compound 1h (1.28 g, 4.23 mmol) and DCI (366 mg, 3.10 mmol) were added, and the reaction mass was stirred at 0° C. for 0.5 h. After concentration under reduced pressure, the residue was separated by silica gel column chromatography (petroleum ether:ethyl acetate=10:1, 0.1% TEA) to obtain the title compound E1-P1 (700 mg, yield 28.2%).

m/z: ES+ [M+H]+732.5

¹H NMR (400 MHz DMSO-d₆) δ 7.42-7.39 (m, 2H), 7.32-7.19 (m, 7H), 6.89-6.85 (m, 4H), 3.92-3.38 (m. 14H), 3.16-2.99 (m, 2H), 2.88-2.80 (m, 1H), 2.76-2.73 (m, 1H), 2.61-2.54 (m, 2H), 2.22-2.10 (m, 2H), 2.05-1.98 (m, 1H), 1.71-1.53 (m, 5H), 1.16-0.98 (m, 17H).

### 8. Preparation of Compound E1-P2

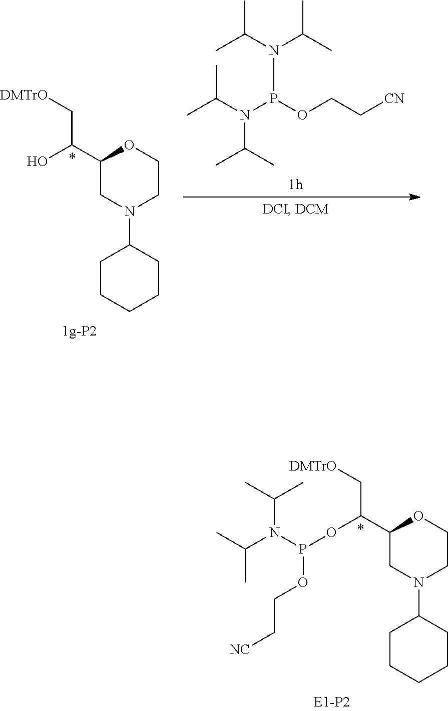

To a solution of compound 1g-P2 (1.10 g, 2.07 mmol) in dichloromethane (15 mL), compound 1h (935 g, 3.10 mmol) and DCI (268 mg, 2.28 mmol) were added, and the reaction mass was stirred at 0° C. for 0.5 h. After concentration under reduced pressure, the residue was separated by silica gel column chromatography (petroleum ether:ethyl acetate=10:1, 0.1% TEA) to obtain the title compound E1-P2 (515 mg, yield 26.7%).

m/z: ES+ [M+H]+732.5

¹H NMR (400 MHz DMSO-d₆) δ 7.41-4.39 (m, 2H), 7.32-7.20 (m, 7H), 6.89-6.85 (m, 4H), 3.98-3.88 (m, 1H), 3.83-3.37 (m, 13H), 3.26-3.07 (m, 1H), 3.02-2.96 (m, 1H), 2.78-2.75 (m, 1H), 2.70-2.55 (m, 3H), 2.19-1.95 (m, 3H), 1.71-1.61 (m, 4H), 155-152 (m, 1H), 1.16-0.97 (m, 17H).

### Example 2: Synthesis of Compound E2

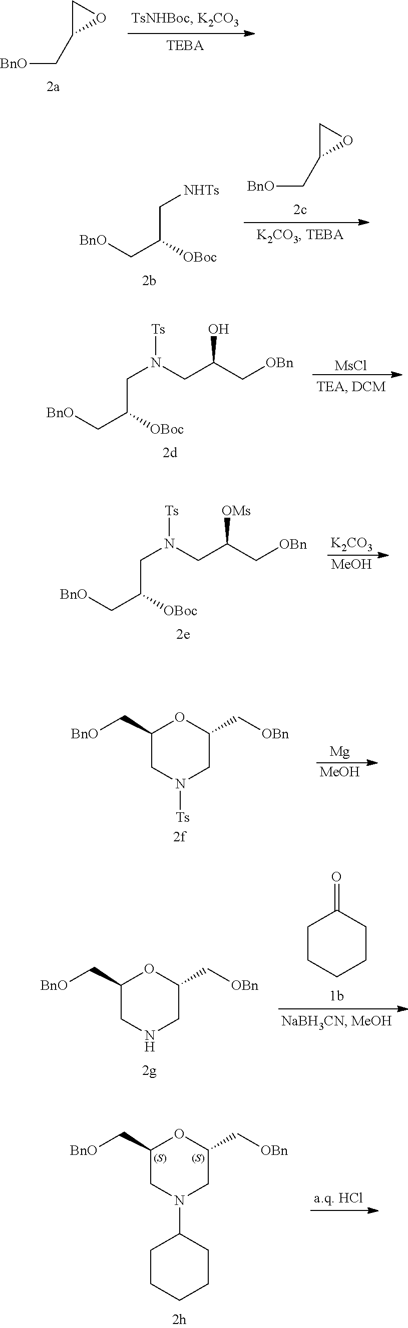

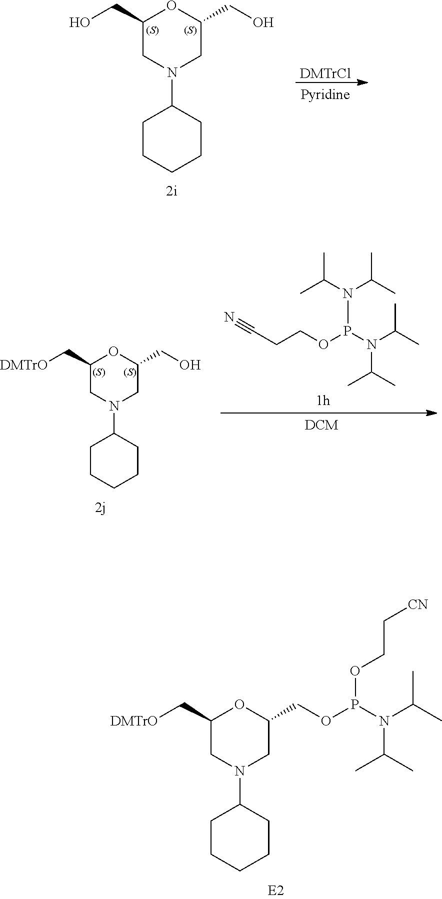

### 1. Preparation of Compound 2b

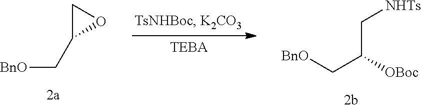

Compound 2a (38.0 g, 231 mmol), TsNHBoc (75.4 g, 278 mmol), K₂CO₃ (6.40 g, 46.3 mmol), and TEBA (triethyl benzyl ammonium chloride, 5.27 g, 23.1 mmol) were added to a three-necked flask at room temperature. The reaction was performed at 95° C. for 2 h, and then the temperature was cooled to 25° C. until TLC (PE/EA=2/1, UV 254 nm) showed that the reaction was complete. The reaction mass was diluted with water (500 mL), and extracted with dichloromethane (200 mL×3). The organic phase was concentrated under reduced pressure, and the crude product was purified by column separation (PE/EA=10/1-3/1) to obtain the title compound 2b (40.0 g).

¹H NMR (400 MHz, CDCl₃) δ 7.72 (d, J=8.4 Hz, 2H), 7.28-7.39 (m, 7H), 4.72-4.86 (m, 2H), 4.42-4.56 (m, 2H), 3.52-3.61 m, 2H), 3.14-3.39 (m, 2H), 2.43 (s, 3H), 1.47 (s, 9H)

### 2. Preparation of Compound 2d

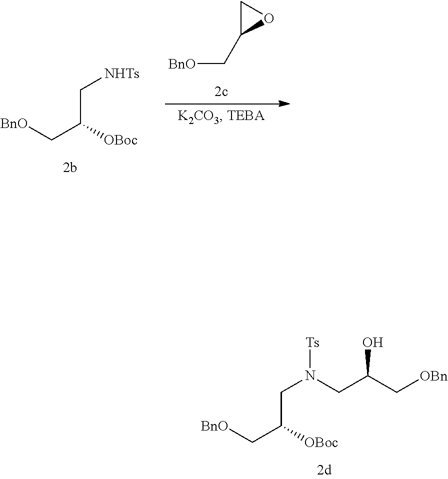

Compound 2b (79.0 g, 181 mmol), compound 2c (24.7 g, 150.8 mmol), potassium carbonate (4.21 g, 30.5 mmol), and TEBA (3.47 g, 15.2 mmol) were reacted at 95° C. for 3 h until TLC (PE/EA=2/1, UV 254 nm) showed that the reaction was complete. The reaction mass was diluted with water (500 mL), and extracted with dichloromethane (200 mL×3). The organic phase was concentrated under reduced pressure, and the crude product was purified by column separation (PE/EA=10/1-3/1) to obtain the title compound 2d (40.0 g, yield 43.8%).

1H NMR (400 MHz, CDCl3) δ 7.64-7.77 (m, 2H), 7.27-7.38 (m, 13H), 5.05-5.16 (m, 1H), 4.50-4.59 (m, 4H), 4.03-4.11 (m, 1H), 3.62-3.76 (m, 2H), 3.44-3.58 (m, 3H), 3.25-3.33 (m, 2H), 3.15-3.20 (m, 1H), 2.42 (s, 3H), 1.46 (s, 9H)

### 3. Preparation of Compound 2e

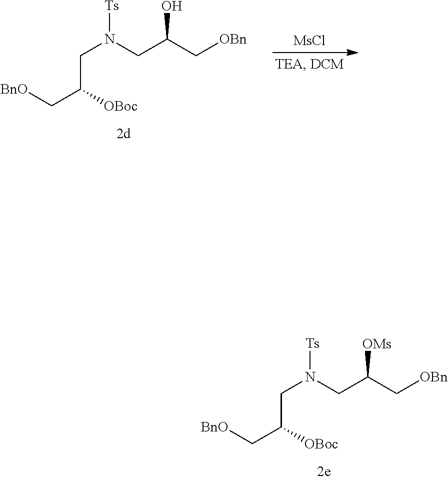

Compound 2d (77.0 g, 128 mmol) and triethylamine (28.6 mL, 205 mmol) were sequentially added to dichloromethane (800 mL) in an ice-water bath, and methanesulfonyl chloride (24.2 g, 212 mmol) was slowly added dropwise. The reaction was performed at 25° C. for 2 h until LCMS showed that the reaction was complete. The reaction mass was washed with water (800 mL), and the organic phase was concentrated under reduced pressure to obtain the crude title compound 2e (90.0 g).

m/z: ES+ [M+Na]+700.1

¹H NMR (400 MHz, CDCl₃) δ 7.68 (d, J=8.4 Hz, 2H), 7.28-7.39 (m, 12H), 5.10-5.20 (m, 1H), 4.93-5.02 (m, 1H), 4.52-4.61 (m, 4H), 3.72-3.88 (m, 2H), 3.52-3.67 (m, 4H), 3.27-3.36 (m, 1H), 3.15-3.22 (m, 1H), 3.04 (s, 3H), 2.43 (s, 3H), 1.43 (s, 9H).

### 4. Preparation of Compound 2f

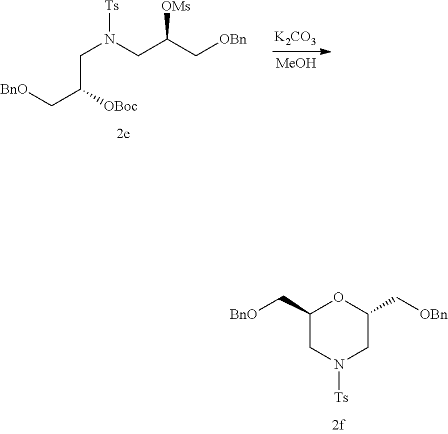

Compound 2e (90.0 g, 133 mmol) and potassium carbonate (91.8 g, 664 mmol) were added to methanol (900 mL) at room temperature. The reaction was performed at 66° C. for 2 h until TLC (PE/EA=2/1, UV 254 nm) showed the formation of new spots. The organic phase was concentrated under reduced pressure. The reaction mass was diluted with water (200 mL), and extracted with dichloromethane (200 mL×3). The organic phase was concentrated under reduced pressure, and the crude product was purified by column separation (PE/EA=30/1-2/1) to obtain the title compound 2f (64.0 g, yield 99.9%).

¹H NMR (400 MHz, CDCl₃) δ 7.62 (d, J=8.4 Hz, 2H), 7.27-7.39 (m, 12H), 4.48-4.62 (m, 4H), 3.93-4.08 (m, 2H), 3.55-3.69 (m, 4H), 2.84-3.10 (m, 4H), 2.44 (s, 3H)

### 5. Preparation of Compound 2g

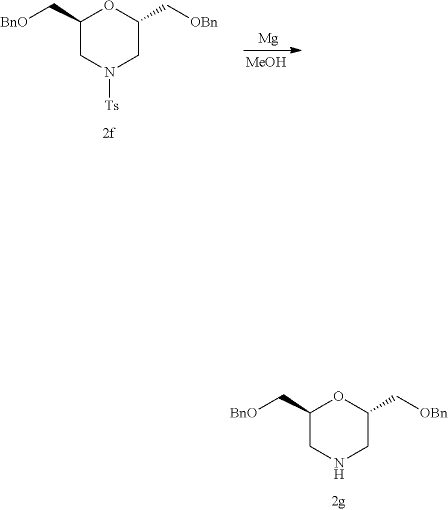

Compound 2f (69.0 g, 143 mmol) and magnesium turnings (54.7 g, 2.28 mol) were added to methanol (400 mL) at room temperature. The reaction was performed at 66° C. for 1 h until TLC (DCM/MeOH=10/1, UV 254 nm) showed that the starting material reaction was complete and new spots were formed. The reaction mass was diluted with water (3,000 mL) and saturated aqueous ammonium chloride solution (3,000 mL), and extracted with dichloromethane (1,000 mL×3). The organic phase was washed with saturated sodium bicarbonate (300 ml×3) and concentrated under reduced pressure to obtain the crude title compound 2g (32.0 g).

¹H NMR (400 MHz, CDCl₃) δ 7.27-7.40 (m, 10H), 4.57 (s, 4H), 3.90-4.00 (m, 2H), 3.59-3.69 (m, 4H), 2.77-3.04 (m, 4H)

### 6. Preparation of Compound 2h

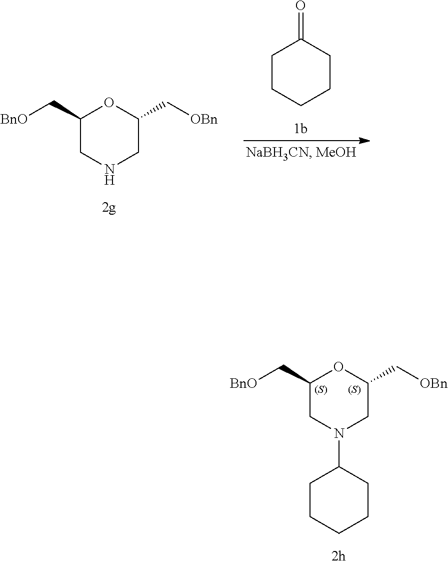

Compound 2g (3.00 g, 9.16 mmol), compound 1b (1.90 mL, 18.3 mmol), and acetic acid (1.01 mL, 18.3 mmol) were added to methanol (30 mL) at room temperature. The reaction was performed at 25° C. for 18 h, followed by the addition of sodium cyanoborohydride (2.30 g, 36.7 mmol) for further reaction at 50° C. for 4 h until TLC (DCM/MeOH=10/1) showed the formation of new spots. The reaction mass was diluted with water (50 mL), and extracted with dichloromethane (30 mL×3). The organic phase was concentrated under reduced pressure, and the crude product was purified by column chromatography (dichloromethane/methanol=99/1-5/1) to obtain the title compound 2h (3.00 g, yield 79.9%).

¹H NMR (400 MHz, CDCl3) δ 7.28-7.41 (m, 10H), 4.51-4.71 (m, 4H), 4.00-4.35 (m, 2H), 3.49-3.77 (m, 4H), 2.69-2.98 (m, 2H), 1.63-2.12 (m, 7H), 1.16-1.44 (m, 6H)

### 7. Preparation of Compound 2i

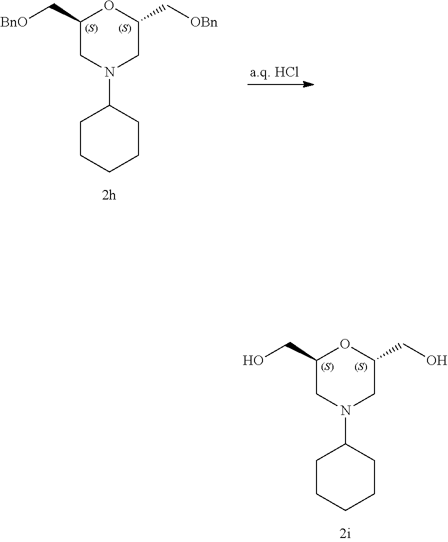

Compound 2h (3.00 g, 7.32 mmol) was added to concentrated hydrochloric acid (10 mL, 12 M) at room temperature. The reaction was performed at 50° C. for 18 h until TLC (DCM/MeOH=10/1) showed the formation of new spots. The reaction mass was concentrated under reduced pressure to obtain the crude title compound 2i (2.00 g).

¹H NMR (400 MHz, CD3OD) δ 4.27-4.38 (m, 1H), 4.11-4.20 (m, 1H), 3.96-4.04 (m, 2H), 3.60-3.68 (m, 3H), 3.36-3.43 (m, 1H), 3.19-3.29 (m, 2H), 3.11 (s, 1H), 2.02-2.17 (m, 2H), 1.90-2.00 (m, 2H), 1.69-1.78 (m, 1H), 1.57-1.69 (m, 1H), 1.35-1.52 (m, 3H), 1.20-1.31 (m, 1H)

### 8. Preparation of Compound 2j

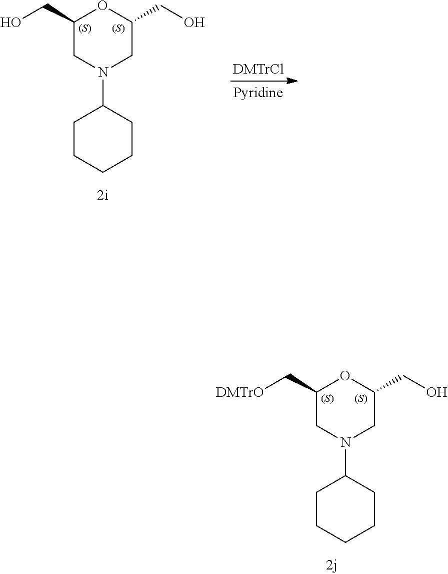

Compound 2i (2.00 g, 8.72 mmol) was dissolved in pyridine (40 mL) at room temperature, and then DMTrCl (2.96 g, 8.72 mmol) was added. The reaction was performed at 25° C. for 18 h until TLC (PE/EA=1/1, UV 254 nm) showed that the reaction was complete. The reaction mass was concentrated under reduced pressure, diluted with saturated aqueous ammonium chloride solution (200 mL), and extracted with DCM (100 mL×3). The organic phase was concentrated under reduced pressure, and the crude product was purified by column chromatography (dichloromethane/methanol=99/1-10/1) to obtain the title compound 2j (1.40 g, yield 30.2%).

### 9. Preparation of Compound E2

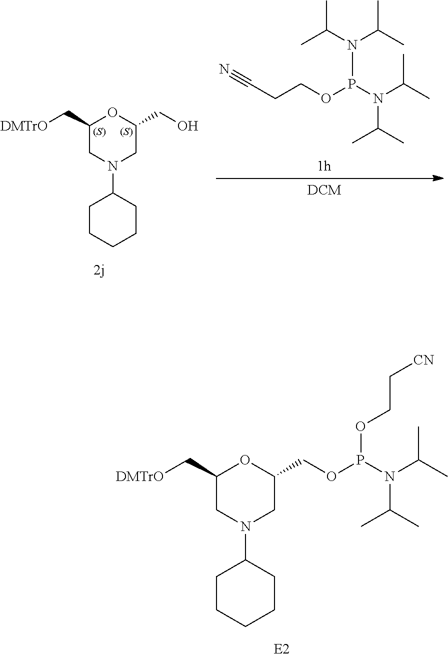

Compound 2j (660 mg, 1.24 mmol), compound 1h (374 mg, 1.24 mmol), and DCI (73.3 mg, 0.621 mmol) were added to DCM (10 mL). The reaction was performed at 25° C. for 1 h until TLC (PE/EA=5/1, PMA) showed that the starting material reaction was complete. The reaction mass was diluted with saturated sodium bicarbonate (50 mL), and extracted with DCM (30 mL×3). The organic phase was concentrated under reduced pressure, and the crude product was separated on a column (PE/EA=99/1-30/1) to obtain the title compound E2 (350 mg, yield 19.3%).

m/z: ES+ [M+H]+732.2

¹H NMR (400 MHz, CDCl₃) δ 7.40-7.50 (m, 2H), 7.28-7.37 (m, 6H), 7.21 (d, J=7.2 Hz, 1H), 6.77-6.89 (m, 4H), 3.67-4.07 (m, 11H), 3.56-3.66 (m, 2H), 3.04-3.33 (m, 2H), 2.30-2.96 (m, 6H), 2.10-2.28 (m, 1H), 1.63-1.88 (m, 4H), 1.47-1.52 (m, 1H), 1.24-1.31 (m, 3H), 1.15-1.23 (m, 12H), 1.11-1.14 (m, 1H)

### Example 3: Synthesis of Compounds E3-P1 and E3-P2

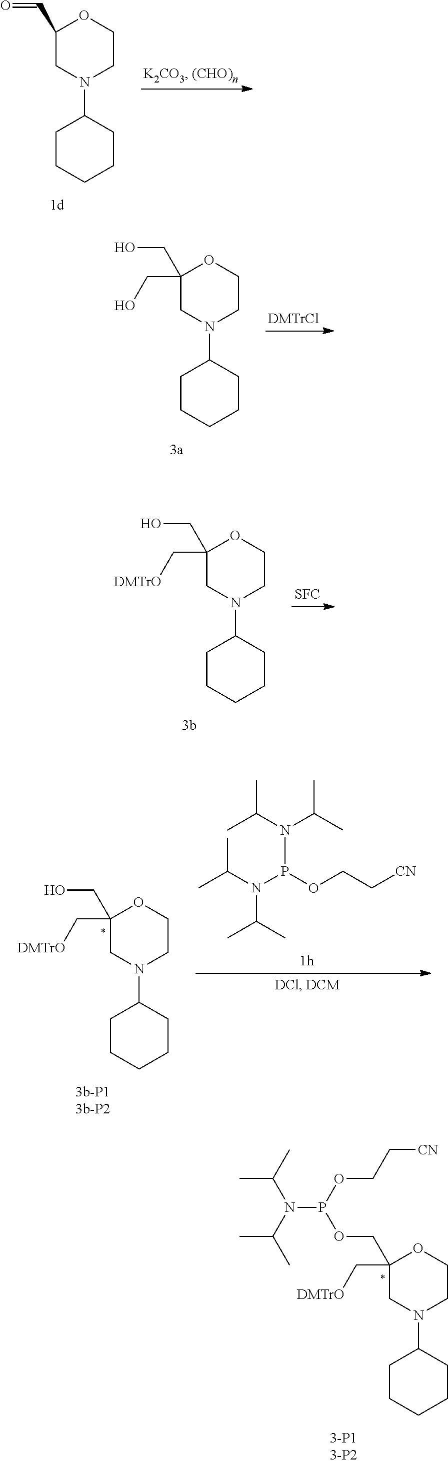

### 1. Preparation of Compound 3a

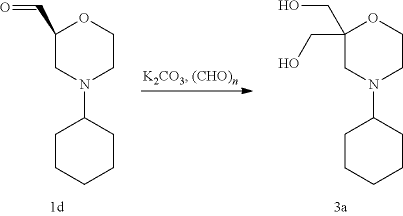 [SMILES: O=C[C@@H]1CN(C2CCCCC2)CCO1 ; OCC1(CO)CN(C2CCCCC2)CCO1]

To a solution of compound 1d (12.1 g, 61.3 mmol) in methanol (85 mL), potassium carbonate (42.3 g, 306.0 mmol) and paraformaldehyde (8.2 g) were added, heated to 70° C., and stirred for 12 h. The reaction mass was cooled to room temperature and filtered. The filtrate was concentrated under reduced pressure, and the residue was separated by silica gel column chromatography (dichloromethane:methanol=10:1) to obtain compound 3a (6.70 g, yield 29.2%).

¹H NMR (400 MHz CDCl₃) δ 4.02-3.99 (m, 2H), 3.66 (s, 4H), 2.72 (s, 2H), 2.61-2.59 (m, 2H), 2.28 (br, 1H), 1.82-1.78 (m, 4H), 1.64-1.61 (m, 1H), 1.28-1.09 (m, 5H)

m/z: ES+ [M+H]+230.1

### 2. Preparation of Compound 3b

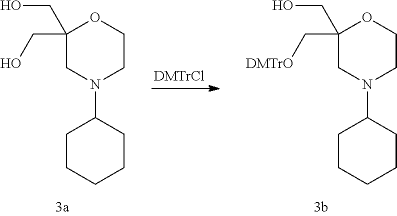

Pyridine (1.72 g, 21.80 mmol) and DMTrCl (775 mg, 2.29 mmol) were added to compound 3a (500 mg, 2.18 mmol). The reaction mass was stirred at room temperature for 1 h. The reaction was quenched with methanol. After multiple batches were combined and concentrated under reduced pressure, the residue was separated by silica gel column chromatography (petroleum ether:ethyl acetate=10:1) to obtain compound 3b (2.20 g, yield 30.4%).

¹H NMR (400 MHz CDCl₃) δ 7.43 (d, J=7.2 Hz, 2H), 7.33-7.27 (m, 5H), 7.23-7.19 (m, 1H), 6.83 (d, J=8.4 Hz, 4H), 4.03-3.94 (m, 3H), 3.80-3.76 (m, 7H), 3.69-3.65 (m, 1H), 3.21 (d, J=8.8 Hz, 1H), 3.05 (d, J=8.4 Hz, 1H), 2.81 (d, J=11.6 Hz, 1H), 2.56-2.44 (m, 3H), 2.27-2.23 (m, 1H), 1.80-1.78 (m, 4H), 1.65-1.61 (m, 1H), 1.26-1.09 (m, 5H)

3. Preparation of Compounds 3b-P1 and 3b-P2

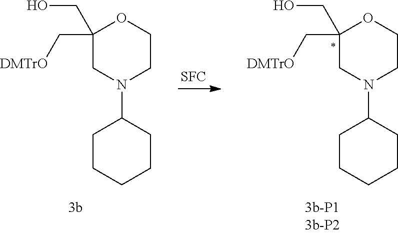

(2-((bis(4-methoxyphenyl)(phenyl) methoxy)methyl)-4-cyclohexylmorpholin-2-yl) methanol (4.10 g, 7.71 mmol) was separated by SFC using a chiral column (column: DAICEL CHIRALPAK AD (250 mm*30 mm, 10 μm); mobile phase: [0.1% NH₃H₂O in IPA]; B %: 50%-50%, 7 min) to obtain 3b-P1 (2.00 g) and 3b-P2 (1.90 g).

3b-P1 spectral data:

¹H NMR (400 MHz CDCl₃) δ 7.44-7.42 (m, 2H), 7.33-7.27 (m, 5H), 7.27-7.21 (m, 1H), 6.84 (d, J=8.8 Hz, 4H), 4.03-3.95 (m, 2H), 3.79 (s, 6H), 3.76-3.67 (m, 1H), 3.22 (d, J=8.4 Hz, 1H), 3.05 (d, J=8.8 Hz, 1H), 2.80 (d, J=11.6 Hz, 1H), 2.56-2.47 (m, 3H), 2.25-2.27 (m, 1H), 1.81-1.78 (m, 4H), 1.65-1.61 (m, 1H), 1.26-1.12 (m, 5H).

HPLC: retention time 1.389 min (Chiralpak AD-3, 50×4.6 mm I.D., 3 μm; mobile phase: A: CO₂, B: IPA (0.1% IPA, v/v); gradient: 0-0.2 min 5% B, 0.2-1.2 min 5-50% B, 1.2-2.2 min 50% B, 2.2-2.6 min 50%-5% B, 2.6-3.0 min 5% B; flow rate: 3.4 mL/min; column temperature: 35° C.)

3b-P2 spectral data:

¹H NMR (400 MHz CDCl₃) δ 7.44-7.42 (m, 2H), 7.33-7.27 (m, 5H), 7.27-7.21 (m, 1H), 6.83 (d, J=8.8 Hz, 4H), 4.03-3.93 (m, 3H), 3.79 (s, 6H), 3.76-3.68 (m, 1H), 3.22 (d, J=8.4 Hz, 1H), 3.06 (d, J=8.8 Hz, 1H), 2.81 (d, J=11.2 Hz, 1H), 2.56-2.47 (m, 3H), 2.25-2.27 (m, 1H), 1.81-1.78 (m, 4H), 1.65-1.62 (m, 1H), 1.26-1.12 (m, 5H).

HPLC: retention time 1.559 min (Chiralpak AD-3, 50×4.6 mm I.D., 3 μm; mobile phase: A: CO₂, B: IPA (0.1% IPA, v/v); gradient: 0-0.2 min 5% B, 0.2-1.2 min 5-50% B, 1.2-2.2 min 50% B, 2.2-2.6 min 50%-5% B, 2.6-3.0 min 5% B; flow rate: 3.4 mL/min; column temperature: 35° C.)

### 4. Preparation of Compound E3-P1

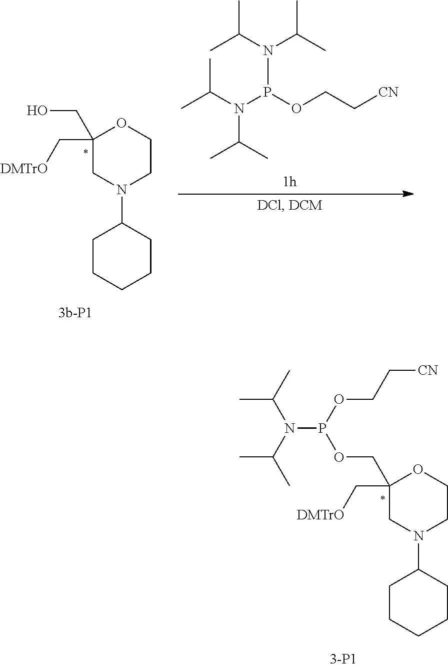

To a solution of compound 3b-P1 (1.60 g, 3.01 mmol) in dichloromethane (16 mL), compound 1h (1.36 g, 4.51 mmol) and DCI (390 mg, 3.31 mmol) were added, and the reaction mass was stirred at 0° C. for 0.5 h. After concentration under reduced pressure, the residue was separated by silica gel column chromatography (petroleum ether:ethyl acetate=10:1, 0.1% TEA) to obtain the compound E3-P1 (1.00 g, yield 43.8%).

¹H NMR (400 MHz DMSO-d₆) δ 7.41-7.38 (m, 2H), 7.31-7.19 (m, 7H), 6.87 (d, J=8.4 Hz, 4H), 4.01-3.89 (m, 1H), 3.73 (s, 6H), 3.68-3.43 (m, 7H), 3.31-3.16 (m, 1H), 2.98 (t, J=9.6 Hz, 1H), 2.72-2.63 (m, 2H), 2.45-2.29 (m, 4H), 2.10-2.07 (m, 1H), 1.63-1.50 (m, 5H), 1.13-1.04 (m, 18H).

### 5. Preparation of Compound E3-P2

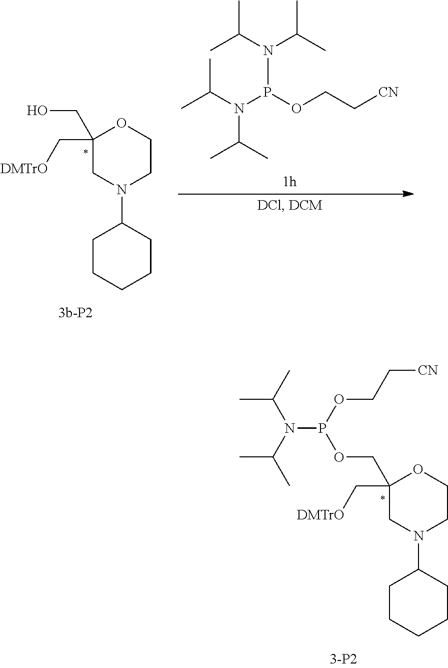

To a solution of compound 3b-P2 (1.50 g, 2.82 mmol) in dichloromethane (15 mL), compound 1h (1.28 g, 4.23 mmol) and DCI (366 mg, 3.10 mmol) were added, and the reaction mass was stirred at 0° C. for 0.5 h. After concentration under reduced pressure, the residue was separated by silica gel column chromatography (petroleum ether:ethyl acetate=10:1, 0.1% TEA) to obtain the compound E3-P2 (1.00 g, yield 47.8%).

¹H NMR (400 MHz DMSO-d₆) δ 7.42-7.39 (m, 2H), 7.30-7.18 (m, 7H), 6.86 (d, J=8.4 Hz, 4H), 4.03-3.91 (m, 1H), 3.72-3.44 (m, 13H), 3.32-3.17 (m, 1H), 2.99 (t, J=9.6 Hz, 1H), 2.72-2.63 (m, 2H), 2.46-2.30 (m, 4H), 2.09-2.06 (m, 1H), 1.63-1.49 (m, 5H), 1.13-1.04 (m, 18H).

### Example 4: Synthesis of siRNA

The siRNA of the present invention was prepared using the solid-phase phosphoramidite method well known in the art. The specific methods can be referred to, for example, PCT Publication Nos. WO2016081444 and WO2019105419, and are briefly described below.

1. Preparation of siRNA with No Ligand Linked to the 3′ End of the Sense Strand

### 1.1 Synthesis of Sense Strand (SS)

Using the solid-phase phosphoramidite method, a blank CPG solid support was used as the starting cycle, and nucleoside monomers were linked one by one according to the arrangement of sense strand nucleotides from the 3′ to 5′ direction. Each linkage of a nucleoside monomer involved four steps of deprotection, coupling, capping, and oxidation or thiolation to synthesize 5 μmol oligonucleotide. The synthesis conditions were as follows:

Nucleoside monomers were provided in a 0.05 mol/L acetonitrile solution. The conditions for each step were identical: 25° C.; deprotection for 3 times using a 3% trichloroacetic acid-dichloromethane solution; coupling twice using a 0.25 mol/L 5-(ethylthio)-1H-tetrazole (ETT)-acetonitrile solution as an activator; capping twice using 10% acetic anhydride-acetonitrile and pyridine/N-methylimidazole/acetonitrile (10:14:76, v/v/v); oxidation twice using 0.05 mol/L of iodine in tetrahydrofuran/pyridine/water (70:20:10, v/v/v); thiolation twice using 0.2 mol/L phenylacetyl disulfide (PADS) in acetonitrile/3-methylpyridine (1:1, v/v).

### 1.2 Synthesis of Antisense Strand (AS)

Using the solid-phase phosphoramidite method, a blank CPG solid support was used as the starting cycle, and nucleoside monomers were linked one by one according to the arrangement of antisense strand nucleotides from the 3′ to 5′ direction. Each linkage of a nucleoside monomer involved four steps of deprotection, coupling, capping, and oxidation or thiolation. The conditions for the synthesis of a 5 μmol oligonucleotide for the antisense strand were identical to those for the sense strand.

### 1.3 Purification and Annealing of Oligonucleotides

### 1.3.1 Ammonolysis

The synthesized solid support (sense or antisense strand) was transferred to a 5 mL centrifuge tube, followed by the addition of 3% diethylamine/ammonia (v/v). The mixture was allowed to react in a thermostatic water bath at 35° C. (or 55° C.) for 16 h (or 8 h), and then filtered. The solid support was washed three times with ethanol/water, 1 mL each time. The filtrate was concentrated by centrifugation, and the crude product was purified.

### 1.3.2 Purification

The methods for purification and desalting are well known to those skilled in the art. For example, a strong anionic packing column can be used; a sodium chloride-sodium hydroxide system can be used for elution and purification. The product can be collected in tubes and desalted using a gel packing purification column. The elution system can be pure water.

### 1.3.3 Annealing

The sense strand (SS) was mixed with the antisense strand (AS) were mixed at a molar ratio (SS/AS=1/1.05) according to the instructions. The mixture was heated in a water bath to 70-95° C. for 3-5 min, and then allowed to cool naturally to room temperature. The system was freeze-dried to obtain the product.

### Example 5: In Vivo Activity

C57BL/6 mice (male, 18-21 g, 6-8 weeks) were randomized. The dose for each animal was calculated based on body weight and administered subcutaneously as a single dose. The siRNA conjugate was administered in a 1 mg/mL solution (0.9% aqueous sodium chloride solution as the solvent). Specifically, before the experiment, the siRNA conjugate was dissolved and diluted to the desired concentration and volume with 0.9% aqueous sodium chloride solution. The dose volume was 5 mL/kg for both the Saline (control) and siRNA conjugate.

The test compound was administered subcutaneously at a single dose of 1 mg/kg on DO, as shown in Table 1.

TABLE 1
Dosing Details for In Vivo Activity Screening

| | | | | | Route and |
|---|---|---|---|---|---|
| | | No. of | | | Frequency of |
| Group | siRNA | Animals | Dose | Volume | Administration |
| Vehicle | Saline | 9 | 1 | 5 | SC, single dose |
| | | | mg/kg | mL/kg | |
| TA01 | DR002346 | 9 | 1 | 5 | SC, single dose |
| | | | mg/kg | mL/kg | |
| TA02 | DR002349 | 9 | 1 | 5 | SC, single dose |
| | | | mg/kg | mL/kg | |
| TA03 | DR002350 | 9 | 1 | 5 | SC, single dose |
| | | | mg/kg | mL/kg | |
| TA04 | DR002351 | 9 | 1 | 5 | SC, single dose |
| | | | mg/kg | mL/kg | |
| TA05 | DR002352 | 9 | 1 | 5 | SC, single dose |
| | | | mg/kg | mL/kg | |
| TA06 | DR002354 | 9 | 1 | 5 | SC, single dose |
| | | | mg/kg | mL/kg | |
| TA07 | DR002355 | 9 | 1 | 5 | SC, single dose |
| | | | mg/kg | mL/kg | |
| TA08 | DR002356 | 9 | 1 | 5 | SC, single dose |
| | | | mg/kg | mL/kg | |

Animals were sacrificed on Days 14, 31, and 56 post-dose (i.e., D14, D31, and D56), and 10 mg of liver tissue was placed in RNAlater solution and stored at −80° C. for subsequent RNA extraction from liver tissue and detection of target gene (mTTR) by qPCR.

The method for qPCR detection is known in the art, and the primer sequences used are shown in Table 2. For example, according to the operating instructions of the High-throughput Tissue RNA Extraction Kit (FireGen, FG0412), cellular RNA was extracted using a nucleic acid extractor (Hangzhou Allsheng Instruments Co., Ltd., Auto-pure96). Reverse transcription was performed using the PrimeScript™ II Ist Strand cDNA Synthesis Kit (Takara, 6210B). Fluorescence quantitative PCR reaction was performed using the TaqMan™ Fast Advanced Master Mix (ABI, 4444965) in a 20 μL system (ABI, QuantStudio3).

TABLE 2
qPCR Primer Sequences

| | | Modifications |
|---|---|---|
| | | at 5′ and 3′ |
| Primer Name | Sequence (5′ to 3′) | Ends |
| mTTR-PF1 | GGGAAGACCGCGGAGTCT (SEQ ID NO: 25) | |
| mTTR-PR1 | CAGTTCTACTCTGTACACTCCTTCTACAAA | |
| | (SEQ ID NO: 26) | |
| mTTR-P1 | CTGCACGGGCTCACCACAGATGA (SEQ ID NO: 27) | 5′6-FAM, |
| | | 3′BHQ1 |
| mGAPDH-PF2 | CGGCAAATTCAACGGCACAG (SEQ ID NO: 28) | |
| mGAPDH-PR2 | CCACGACATACTCAGCACCG (SEQ ID NO: 29) | |
| mGAPDH-P2 | ACCATCTTCCAGGAGCGAGACCCCACT (SEQ ID NO: 30) | 5′TET, 3′BHQ2 |

### Data Statistics and Analysis

The 2^{-ΔΔCt} value was calculated and converted into a percentage to obtain the residual inhibition;

$$ΔΔ Ct = [ ( {Ct}_{target gene of experimental group} - {Ct}_{internal reference of experimental group} ) - ( {Ct}_{target gene of control group} - {Ct}_{internal reference of control group} ) ] .$$

The target gene was mTTR, and the internal reference was mGAPDH.

The results are shown in Table 3. The experimental results showed that the compounds of the present invention (DR002346, DR002349, DR002350, DR002351 and DR002352) achieved superior target gene inhibition compared with known modifications (DR002354 and DR002355).

TABLE 3
In Vivo Activity Screening Results

| | D14 | | D31 | | D56 | |
|---|---|---|---|---|---|---|
| | mTTR | | mTTR | | mTTR | |
| | Expression | | Expression | | Expression | |
| Compound | Level | SD | Level | SD | Level | SD |
| Saline | 107.7% | 13.3% | 100.4% | 10.2% | 100.5% | 10.4% |
| DR002346 | 2.0% | 0.3% | 4.3% | 1.4% | 31.0% | 6.3% |
| DR002349 | 2.6% | 1.3% | 5.0% | 0.9% | 16.5% | 1.6% |
| DR002350 | 2.0% | 0.7% | 3.2% | 1.2% | 15.4% | 2.9% |
| DR002351 | 2.0% | 0.9% | 8.6% | 3.2% | 48.3% | 14.7% |
| DR002352 | 2.4% | 0.6% | 5.5% | 0.5% | 34.1% | 7.7% |
| DR002354 | 2.8% | 0.3% | 6.5% | 0.6% | 22.1% | 4.1% |
| DR002355 | 1.9% | 0.6% | 6.6% | 0.5% | 26.7% | 3.4% |
| DR002356 | 75.7% | 4.0% | 86.5% | 2.0% | / | / |

### Example 6: Validation of Compounds of the Invention in C₅₇BL/6 Mouse Model

Following the experimental procedures outlined in Example 5, the long-term efficacy of the test compounds (see Table 4) was validated in a C57BL/6 mouse model.

The dose for each animal was calculated based on body weight and administered subcutaneously as a single dose. The siRNA conjugate was administered in a 1 mg/mL solution (0.9% aqueous sodium chloride solution as the solvent). The dose was 1 mpk. Blood samples were collected from the orbit at various time points, and the mTTR protein levels in serum were detected using an ELISA kit (Abcam, ab282297). The experimental results are shown in Table 4.

TABLE 4
Experimental Results of Compounds in C57BL/6 Mouse Model

| | | | Day 14 | | Day28 | | Day42 | | Day56 | |
|---|---|---|---|---|---|---|---|---|---|---|
| | | | Residual | | Residual | | Residual | | alResidual | |
| | | | mTTR | | mTTR | | mTTR | | mTTR | |
| | Compound | Dose | Protein | SD | Protein | SD | Protein | SD | Protein | SD |
| 1 | Saline | / | 100.00% | 10.00% | 100.00% | 8.10% | 100.00% | 6.30% | 100.00% | 4.90% |
| 2 | DR002242 | 1 | 2.80% | 1.40% | 5.80% | 2.90% | 12.00% | 3.50% | 17.70% | 4.90% |
| | | mpk | | | | | | | | |
| 3 | DR005781 | 1 | 1.70% | 0.20% | 4.20% | 0.80% | 6.20% | 1.90% | 12.50% | 2.20% |
| | | mpk | | | | | | | | |
| 4 | DR005782 | 1 | 1.90% | 0.30% | 4.50% | 0.60% | 9.30% | 2.80% | 21.70% | 5.10% |
| | | mpk | | | | | | | | |

### Example 7: Validation of Compounds of the Invention in Mouse HDI Model

### HDI Animal Modeling

Using the tail vein high-pressure injection method, six- to eight-week-old female Balb/c mice were transfected in vivo with a dual-gene stable transfection system. The transfection was performed via the tail vein, injecting a TransIT®-QR delivery solution (total volume: 10% of animal body weight, Mirusbio-MIR 5240) containing Piggy-Bac transposon plasmids (Suzhou Bangye) and Piggy-Bac helper plasmids (Suzhou Bangye) (mass ratio: 1:1, total plasmid amount: 100 μg) containing different mass ratios of target gene cDNA sequences (Genbank accession No. NM 014495.2) into mice within 5-7 seconds using a 27-gauge needle. After injection, the mice were returned to their cage and observed for 30 minutes. The day of modeling was designated as Day 0. Serum samples were collected at various time points after modeling (Day 7 to Day 35) for the detection of SEAP expression levels.

The dual-gene stable transfection system comprised a Piggy-Bac helper plasmid and a Piggy-Bac transposon plasmid, wherein the Piggy-Bac helper plasmid provided the Piggy-Bac transposase, while the Piggy-Bac transposon plasmid was based on the Piggy-Bac transposon and contained a dual-gene expression element, which included the secreted alkaline phosphatase (SEAP) gene and the target gene (ANGPTL3).

### Detection of SEAP Expression Levels

The standard provided with the kit (Phospha-Light™ SEAP Reporter Gene Assay System, Invitrogen, T1016) was diluted 2-fold starting from an initial concentration of 15 mU/mL to obtain 7 concentration points.

The CSPD substrate was mixed with the reaction buffer diluent at a ratio of 1:20 to prepare the reaction solution. The 5× dilution buffer was diluted to 1× dilution buffer with DNAse- and RNAse-free distilled water. The serum was mixed with 1× dilution buffer in a centrifuge tube to prepare the sample dilution, which was then incubated at 65° C. for 30 min and then cooled to room temperature. Approximately 50 μL of the sample dilution was added to a 96-well plate, followed by the addition of 50 μL of assay buffer per well, and incubated at room temperature for 5 min. Then, 50 μL of the reaction solution was added to each well and incubated at room temperature for 20 min. The SEAP chemiluminescence value was read using a microplate reader (Tecan, Infinite 200).

The effect of the compounds on inhibiting the expression of the target gene was evaluated by measuring the SEAP expression levels in serum. A sample to be tested capable of inhibiting SEAP expression levels was selected as a nucleic acid drug.

On Day 15 post-modeling, according to Tables 5 and 6, each mouse was administered with a single subcutaneous injection of 200 AL of Saline containing 3 mg/kg (mpk) RNAi reagent or 200 AL of Saline without RNAi reagent as a vehicle. The results of the HDI model screening are shown in Tables 5 and 6.

TABLE 5
Superior Efficacy of siRNAs Carrying Compounds
of the Invention (DR005690, DR005921, and
DR005922) vs. Positive Compound DR001479

| | Compound | Dose | Day 7 | Day 14 | Day 21 |
|---|---|---|---|---|---|
| 1 | DR005690 | 1 mpk | 7.08% | 3.28% | 5.07% |
| 2 | DR005921 | 1 mpk | 5.88% | 2.52% | 3.28% |
| 3 | DR005922 | 1 mpk | 8.62% | 6.24% | 8.31% |
| 4 | DR001479 | 1 mpk | 19.39% | 13.05% | 16.72% |

TABLE 6
Superior Efficacy of siRNAs Carrying Compounds of the Invention (DR005691) vs.
Positive Compound DR001479 and Comparable Efficacy of siRNA Not Carrying Compounds of
the Invention (DR005687) vs. Positive Compound DR001479

| | Compound | Dose | Day7 | Day14 | Day21 | Day28 | Day35 |
|---|---|---|---|---|---|---|---|
| 1 | DR005687 | 1 mpk | 16.8% | 11.1% | 18.5% | 29.7% | 47.8% |
| 2 | DR005691 | 1 mpk | 17.4% | 8.9% | 9.7% | 17.6% | 25.8% |
| 3 | DR001479 | 1 mpk | 28.6% | 21.3% | 24.4% | 33.8% | 46.6% |

### Example 8: Validation of Compounds of the Invention in Mouse HDI Model

Following the procedures in Example 7, the compounds of the present invention were validated in another HDI mouse model, except that siRNA targeting APOC3 was used in this experiment, while siRNA targeting ANGPTL3 was used in Example 7. The dosing and results are shown in Table 7.

TABLE 7
Comparable Efficacy of siRNA Carrying Compounds of the Invention
(DR005682 and DR005684) vs. DR005681 Carrying Prior Art IB

| | Compound | Dose | Day 7 | Day14 | Day21 | Day28 |
|---|---|---|---|---|---|---|
| 1 | DR005681 | 3 mpk | 11.20% | 13.40% | 23.80% | 41.60% |
| 2 | DR005682 | 3 mpk | 11.60% | 12.80% | 16.00% | 36.50% |
| 3 | DR005684 | 3 mpk | 10.40% | 10.20% | 23.80% | 47.90% |

The above content is a further detailed description of the present invention in conjunction with specific preferred embodiments, but it cannot be regarded that the specific embodiments of the present invention are limited to these descriptions. For a person of ordinary skill in the art to which the present invention belongs, without departing from the idea of the present invention, a number of simple deductions or replacements may be made, which should be regarded as falling within the protection scope of the present invention.

1. A compound of formula (X), or a pharmaceutically acceptable salt, tautomer or stereoisomer thereof: 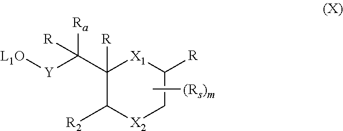
wherein,
X₁ is a bond, O or S;
X₂ is a bond or NR₁;
Y is a bond or [C(Rₐ)(R_{b})]₁-4;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be further optionally deuterated or fully deuterated;
R₂ is selected from H, D or OL₂;
R is selected from H, D, -L-OR′, -L-NR″R′″, R_{b} or —C(OL₂)(R_{c})(R_{d}), wherein R may be optionally deuterated or fully deuterated;
L₁ and L₂ are selected from H, a reactive phosphorus group or a protecting group;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a bond or C₁₋₆ alkylene, wherein L may be optionally deuterated or fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; otherwise R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated or fully deuterated;
n=0, 1, 2, 3, 4, 5, 6, 7, 8, 9 or 10;
p=0 or 1;
Rₛ is selected from H, D, C₁₋₆ alkyl or C₁₋₆ haloalkyl, wherein Rₛ may also be optionally deuterated or fully deuterated;
m=0, 1, 2, 3 or 4;
provided that an OL₂ group is present in either R or R₂.

2. A compound of formula (X) according to claim 1, or a pharmaceutically acceptable salt, tautomer, or stereoisomer thereof, wherein the compound of formula (X) has one or more of the following definitions:
i) wherein X₁ is a bond or O, preferably O;
ii) wherein X₂ is NR₁, and wherein R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, or 5- to 14-membered heteroaryl, preferably from C₃₋₈ cycloalkyl, 3- to 10-membered heterocyclyl, C₆₋₁₀ aryl, or 5- to 14-membered heteroaryl, more preferably from C₃₋₈ cycloalkyl or 3- to 10-membered heterocyclyl, especially C₅₋₆ cycloalkyl;
iii) wherein Y is a bond;
iv) wherein Y is [C(Rₐ)(R_{b})]₁₋₄, preferably C(Rₐ)(R_{b});
v) wherein R₂ is selected from H or D;
vi) wherein one of R is —C(OL₂)(R_{c})(R_{d}), and is optionally deuterated or fully deuterated;
vii) wherein one of L₁ and L₂ is phosphoramidite (preferably —P(OCH₂CH₂CN)(N(iPr)₂)), and the other is dimethoxytrityl (DMTr); and
viii) wherein Rₐ, R_{b}, R_{c}, and R_{d} are independently selected from H, D, halogen, CN, —C₀₋₆ alkyl-OR′, —C₀₋₆ alkyl-NR″R′″, C₁₋₆ alkyl, C₁₋₆ haloalkyl, C₂₋₆ alkenyl, or C₂₋₆ alkynyl, preferably H, D, halogen, CN, C₁₋₆ alkyl, or C₁₋₆ haloalkyl; wherein Rₐ, R_{b}, R_{c}, and R_{d} are optionally deuterated or fully deuterated.

3-9. (canceled)

10. A compound of formula (X) according to claim 1, or a pharmaceutically acceptable salt, tautomer, or stereoisomer thereof, wherein the compound of formula (X) is selected from the following formulas: 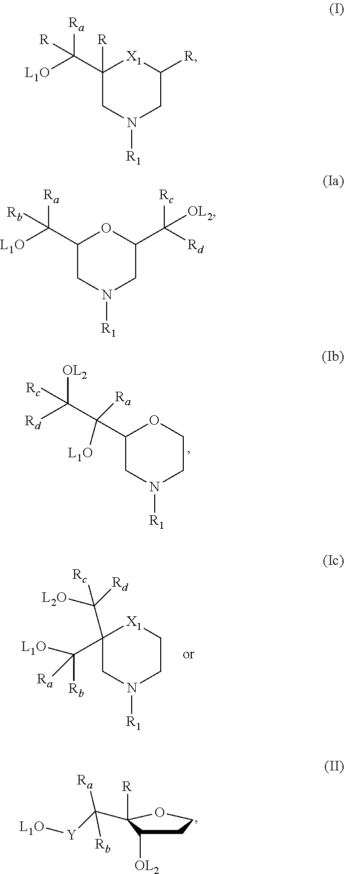
wherein each group is as defined in claim 1.

11. A compound of formula (X) according to claim 10, or a pharmaceutically acceptable salt, tautomer, or stereoisomer thereof, which is a compound of formula (I): 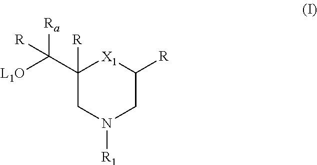
wherein,
X₁ is a bond, O or S;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, which may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
one of R is —C(OL₂)(R_{c})(R_{d}), and the other two are R_{b};
L₁ and L₂ are selected from H, a reactive phosphorus group or a protecting group;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a bond or C₁₋₆ alkylene, which may be optionally deuterated or fully deuterated;
R′, R″ and R′″ are independently selected from H, —C₁₋₆ alkylene —OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), which is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl; wherein Rₓ is optionally deuterated or fully deuterated;
n=0, 1, 2, 3, 4, 5, 6, 7, 8, 9 or 10;
p=0 or 1.

12. A compound of formula (I) according to claim 11, or a pharmaceutically acceptable salt, tautomer, or stereoisomer thereof, wherein,
X₁ is a bond, O or S;
R₁ is cyclohexyl, and is optionally deuterated or fully deuterated;
one of R is —C(OL₂)(R_{c})(R_{d}), and the other two are R_{b};
L₁ and L₂ are selected from DMTr or —P(OCH₂CH₂CN)(N(iPr)₂);
Rₐ, R_{b}, R_{c}, and R_{d} are independently selected from H or D.

13. A compound of formula (X) according to claim 10, or a pharmaceutically acceptable salt, tautomer, or stereoisomer thereof, wherein the compound of formula (X) is a compound of formula (Ia), (Ib), or (Ic): 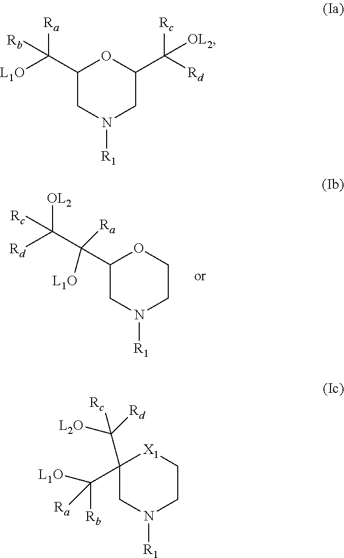
wherein,
X₁ is a bond, O or S;
R₁ is selected from C₁₋₂₀ alkyl, C₁₋₂₀ haloalkyl, C₂₋₂₀ alkenyl, C₂₋₂₀ alkynyl, C₃₋₈ cycloalkyl, 3- to 10-membered heterocyclyl, C₆₋₁₀ aryl, 5- to 14-membered heteroaryl or —[C(O)]ₚ—C₁₋₂₀ alkylene-(OCH₂CH₂)nRx, wherein said group may be optionally substituted with one or more group selected from halogen, CN, OR′, SR′, NR″R′″, C(O)OR′, C(S)OR′, C(O) NR″R′″, C(S) NR″R′″, OC(O)R′, OC(S)R′, NR″C(O)R′″, NR″C(S)R′″, C₁₋₆ alkyl, C₁₋₆ haloalkyl, C₃₋₈ cycloalkyl, 3- to 10-membered heterocyclyl, C₆₋₁₀ aryl or 5- to 14-membered heteroaryl; wherein R₁ may be also optionally deuterated or fully deuterated;
L₁ and L₂ are selected from H, a reactive phosphorus group or a protecting group;
Rₐ, R_{b}, R_{c} and R_{d} are independently selected from H, D, halogen, CN, -L-OR′, -L-NR″R′″, C₁₋₆ alkyl, C₁₋₆ haloalkyl, C₂₋₆ alkenyl, C₂₋₆ alkynyl, -L-C₃₋₈ cycloalkyl or -L-4-membered to -L-7-membered heterocyclyl; wherein Rₐ, R_{b}, R_{c} and R_{d} are optionally deuterated or fully deuterated;
wherein L is a bond or C₁₋₆ alkylene, wherein L may be optionally deuterated until fully deuterated;
R′, R″ and R′″ are independently selected from H, —C₁₋₆ alkylene-OH, —C₁₋₆ alkylene-NH₂, C₁₋₆ alkyl, C₁₋₆ haloalkyl, C₂₋₆ alkenyl, C₂₋₆ alkynyl, —C₀₋₆ alkylene-C₃₋₈ cycloalkyl, —C₀₋₆ alkylene-(3- to 10-membered heterocyclyl), —C₀₋₆ alkylene-C₆₋₁₀ aryl or —C₀₋₆ alkylene-(5- to 14-membered heteroaryl), wherein an aforementioned group is optionally substituted with one or more groups selected from halogen, CN, NO₂, OH, NH₂, C₁₋₆ alkyl or C₁₋₆ haloalkyl; or R″ and R′″ together with the N atom connecting thereto form a 3- to 10-membered heterocyclyl;
Rₓ is selected from H, D, OH, OC₁₋₆ alkyl, C₁₋₆ alkyl, C₁₋₆ haloalkyl, C₂₋₆ alkenyl, C₂₋₆ alkynyl, C₃₋₈ cycloalkyl, 3- to 10-membered heterocyclyl, C₆₋₁₀ aryl or 5- to 14-membered heteroaryl;
wherein Rₓ is optionally deuterated or fully deuterated;
n=0, 1, 2, 3, 4, 5, 6, 7, 8, 9 or 10;
p=0 or 1;
preferably,
X₁ is a bond, O or S;
R₁ is selected from C₃₋₆ cycloalkyl or 5- to 6-membered heterocyclyl, and is optionally deuterated or fully deuterated;
L₁ and L₂ are selected from DMTr or —P(OCH₂CH₂CN)(N(iPr)₂);
Rₐ, R_{b}, R_{c}, and R_{d} are independently selected from H, D, halogen, C₁₋₆ alkyl, or C₁₋₆ haloalkyl, and are optionally deuterated or fully deuterated;
preferably,
X₁ is a chemical bond, O or S;
R₁ is C₄₋₆ cycloalkyl, and is optionally deuterated or fully deuterated;
L₁ and L₂ are selected from DMTr or —P(OCH₂CH₂CN)(N(iPr)₂);
Rₐ, R_{b}, R_{c}, and R_{d} are independently selected from H, D, halogen, C₁₋₄ alkyl, or C₁₋₄ haloalkyl, and are optionally deuterated or fully deuterated;
preferably,
X₁ is a chemical bond or O;
R₁ is cyclohexyl, and is optionally deuterated until fully deuterated;
L₁ and L₂ are selected from DMTr or —P(OCH₂CH₂CN)(N(iPr)₂);
Rₐ, R_{b}, R_{c}, and R_{d} are independently selected from H or D.

14. A compound according to claim 1, or a pharmaceutically acceptable salt, tautomer or stereoisomer thereof, wherein the compound is selected from:

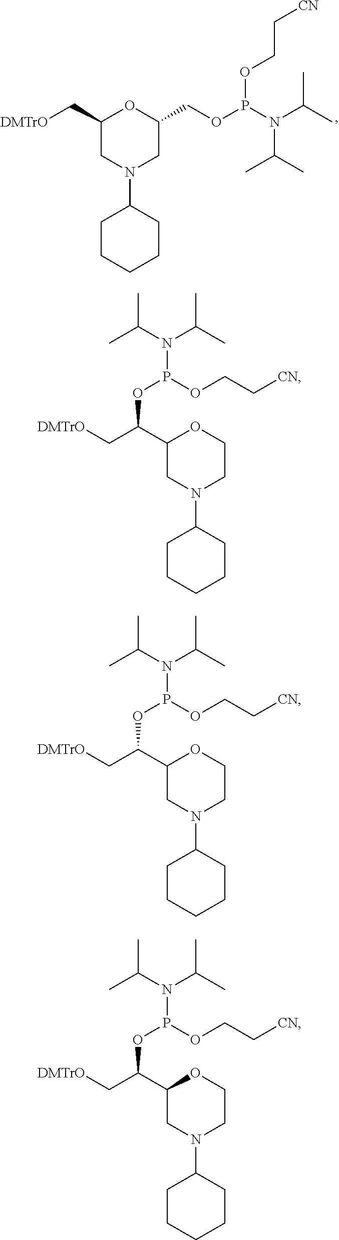

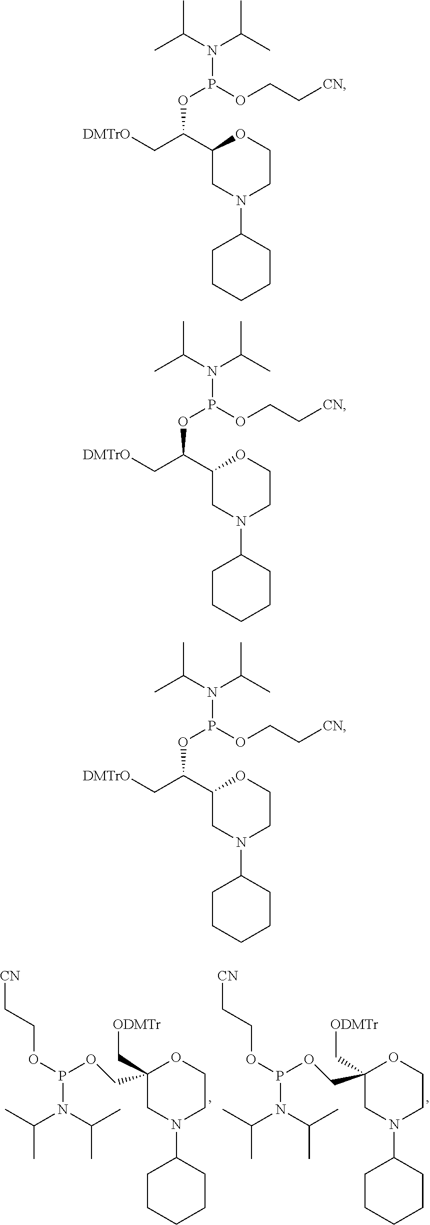

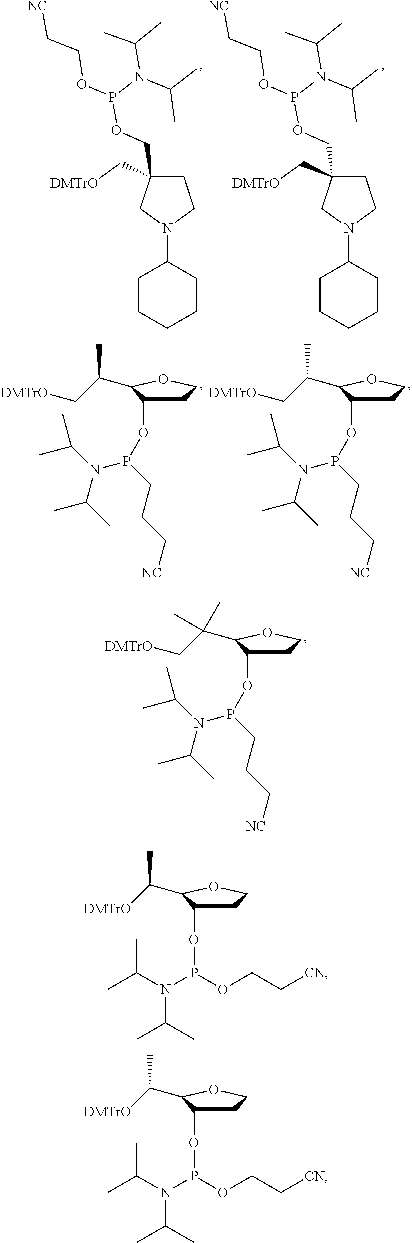

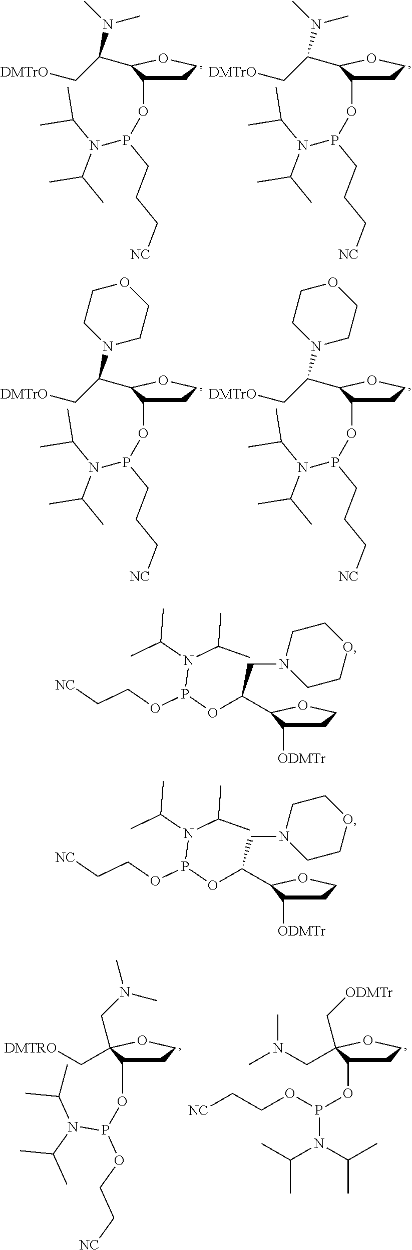

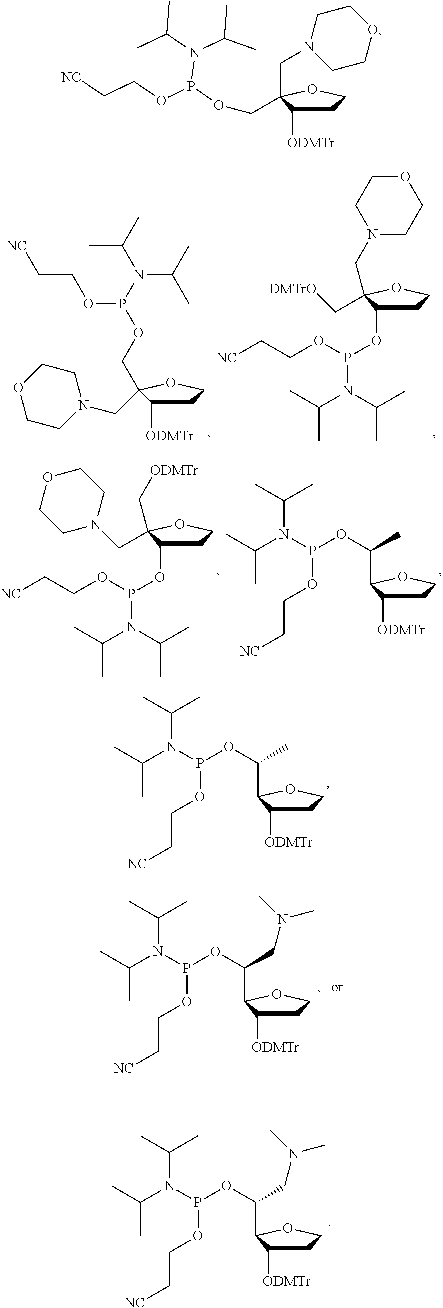

15. An oligonucleotide comprising at the 5′ and/or 3′ end one or more compounds of formula (X′): 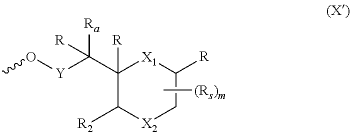
wherein,
R₂ is selected from H, D or O [symbol] ;
R is selected from H, D, -L-OR′, -L-NR″R′″, R_{b} or —C(O [symbol] )(R_{c})(R_{d}), wherein R may be optionally deuterated or fully deuterated;
provided that either of R and R₂ contains an O [symbol] group, wherein [symbol] represents a bond linked to a nucleotide of the oligonucleotide, and [symbol] at the terminal is linked to H, a reactive phosphorus group, or a protecting group; the other groups are as defined in claim 1.

16. An oligonucleotide according to claim 15, wherein the oligonucleotide has any one of the following definitions:
i) wherein the compound of formula (X′) is a compound of: 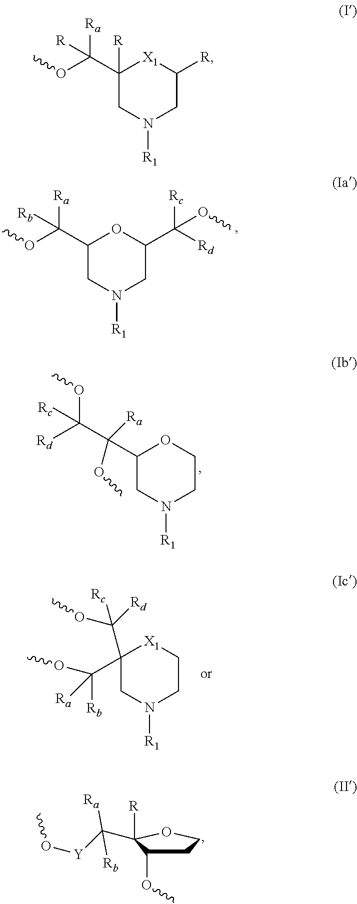
wherein each group is as defined in claim 15; and
ii) wherein the compound of formula (X′) is a compound of: 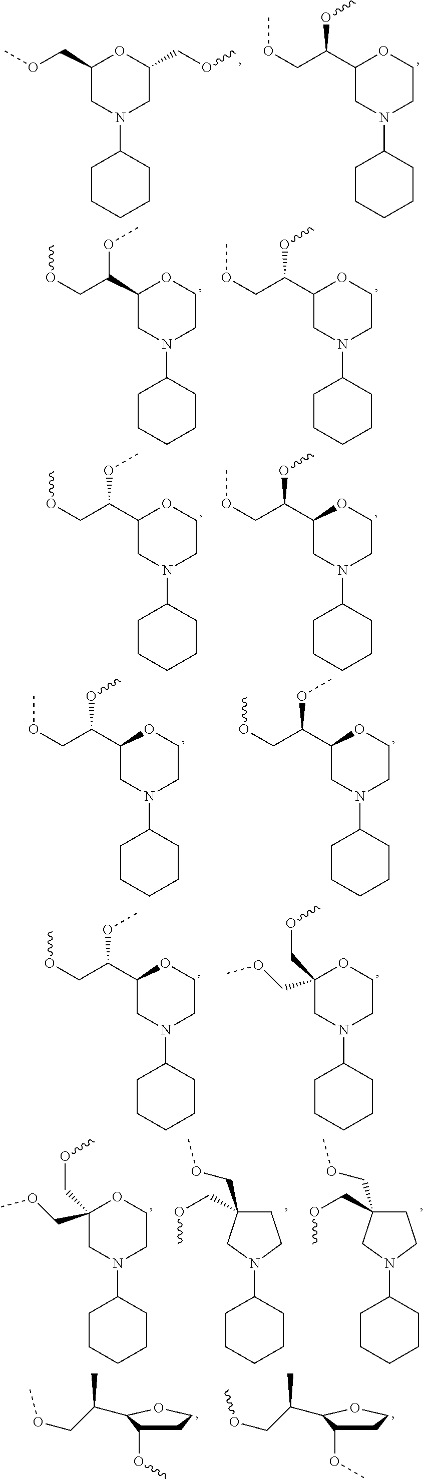 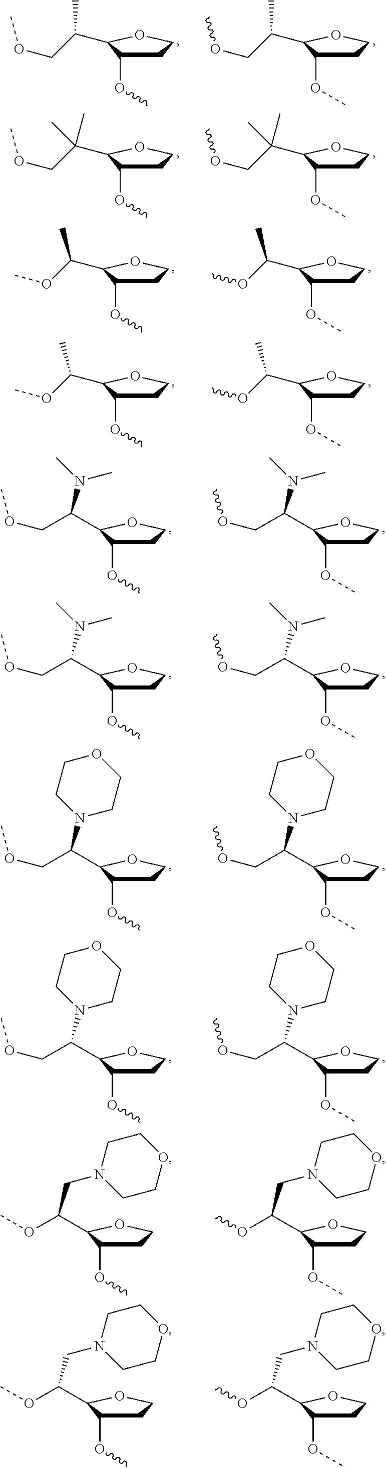 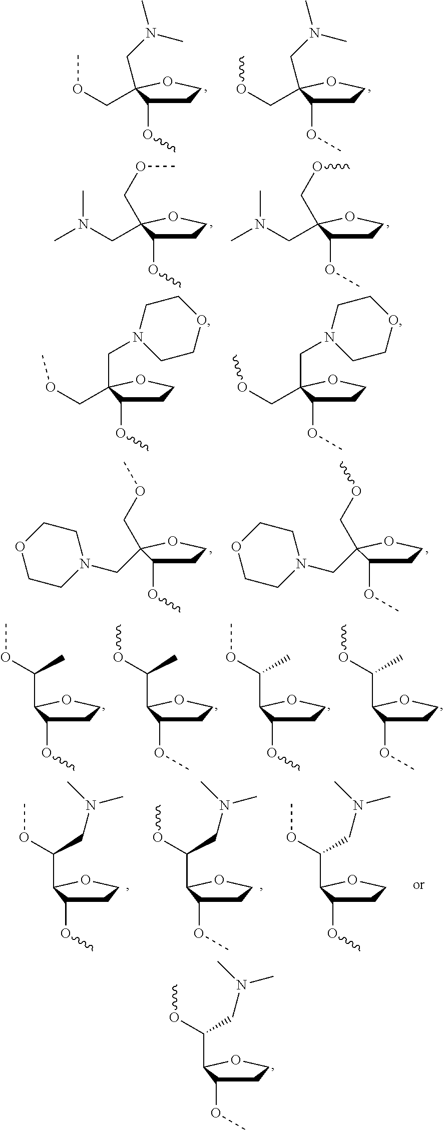
wherein the linkage is made from [symbol] to [symbol] of the compound in the 5′ to 3′ direction.

17. (canceled)

18. A dsRNA, wherein the dsRNA has a sense strand and an antisense strand between which the sequences are substantially complementary, wherein each strand has 14 to 30 nucleotides that are linked via a phosphate group, a phosphorothioate group, or other linker molecule, and wherein the said sense strand has the following structure: 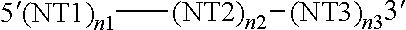
wherein,
NT₁ is a compound of formula (X′) according to claim 15;
NT2 is a modified or unmodified nucleotide;
NT3 is a compound of formula (X′) according to claim 15;
n1 is 0, 1, 2, or 3;
n2 is an integer in the range of 14 to 30;
n3 is 0, 1, 2, or 3;
and n1 and n3 are not both zero (0) simultaneously.

19. A dsRNA according to claim 18, wherein the dsRNA has any one of the following definitions:
i) wherein n1 is 0, NT3 is selected from a compound of formula (I′), (Ia′), (Ib′), or (Ic′), and n3 is 2;
ii) wherein n1 is 2, NT1 is a compound of formula (I′), (Ia′), (Ib′), or (Ic′), NT3 is selected from a compound of formula (I′), (Ia′), (Ib′), or (Ic′), and n3 is 2;
iii) wherein n1 is 1, NT1 is selected from a compound of formula (I′), (Ia′), (Ib′), or (Ic′), NT3 is selected from a compound of formula (I′), (Ia′), (Ib′), or (Ic′), and n3 is 1; and
iv) wherein n1 is 1, NT1 is selected from a compound of formula (I′), (Ia′), (Ib′), or (Ic′), NT3 is selected from a compound of formula (I′), (Ia′), (Ib′), or (Ic′), and n3 is 2.

20-22. (canceled)

23. A dsRNA according to claim 18, wherein the dsRNA is further conjugated with a ligand moiety comprising N-acetylgalactosamine, preferably conjugated with the ligand moiety at the sense strand, preferably conjugated with the ligand moiety at the 3′ end of the sense strand, preferably conjugated with the ligand moiety at the 5′ end of the sense strand.

24. A dsRNA according to claim 23, wherein the ligand comprises one or more GalNAcs.

25. A dsRNA according to claim 24, wherein a ligand comprising GalNAc is conjugated to the 3′ end of the sense strand, and the ligand is selected from L₉₆, GL6, or GL12, preferably GL6.

26. A dsRNA according to claim 18, wherein the dsRNA is selected from small interfering RNA (siRNA) and short hairpin RNA (shRNA).

27. A cell comprising a dsRNA of claim 18.

28. A pharmaceutical composition comprising a dsRNA of claim 18, and optionally a pharmaceutically acceptable vector or excipient.

29. A kit comprising a dsRNA of claim 18.

30. A pharmaceutical composition comprising a cell of claim 27, and optionally a pharmaceutically acceptable vector or excipient.

31. A kit comprising a cell of claim 27.


SEQUENCE LISTING (30 sequences)

SEQ ID NO: 1 - RNA, 19 nt, synthetic construct
  source 1..19: mol_type = other RNA; note = SR004665S
cagtgttcttgctctataa

SEQ ID NO: 2 - RNA, 19 nt, synthetic construct
  source 1..19: mol_type = other RNA; note = SR004670S
cagtgttcttgctctataa

SEQ ID NO: 3 - RNA, 19 nt, synthetic construct
  source 1..19: mol_type = other RNA; note = SR004671S
cagtgttcttgctctataa

SEQ ID NO: 4 - DNA, 21 bp, synthetic construct
  source 1..21: mol_type = other DNA; note = SR004673S
  misc_feature 1..19: note = RNA
  misc_feature 20..21: note = DNA
cagtgttcttgctctataatt

SEQ ID NO: 5 - RNA, 19 nt, synthetic construct
  source 1..19: mol_type = other RNA; note = SR004674S
cagtgttcttgctctataa

SEQ ID NO: 6 - RNA, 19 nt, synthetic construct
  source 1..19: mol_type = other RNA; note = SR004675S
cagtgttcttgctctataa

SEQ ID NO: 7 - RNA, 19 nt, synthetic construct
  source 1..19: mol_type = other RNA; note = SR004668S
cagtgttcttgctctataa

SEQ ID NO: 8 - RNA, 19 nt, synthetic construct
  source 1..19: mol_type = other RNA; note = SR004669S
cagtgttcttgctctataa

SEQ ID NO: 9 - RNA, 21 nt, synthetic construct
  source 1..21: mol_type = other RNA; note = SR004441A
ttatagagcaagaacactgtt

SEQ ID NO: 10 - RNA, 19 nt, synthetic construct
  source 1..19: mol_type = other RNA; note = SR004469S
cagtgttcttgctctataa

SEQ ID NO: 11 - RNA, 19 nt, synthetic construct
  source 1..19: mol_type = other RNA; note = SR011387S
cagtgttcttgctctataa

SEQ ID NO: 12 - RNA, 21 nt, synthetic construct
  source 1..21: mol_type = other RNA; note = SR011388S
aacagtgttcttgctctataa

SEQ ID NO: 13 - RNA, 21 nt, synthetic construct
  source 1..21: mol_type = other RNA; note = SR011286S
tccacgttgcttgaaattgaa

SEQ ID NO: 14 - RNA, 21 nt, synthetic construct
  source 1..21: mol_type = other RNA; note = SR011542S
tccacgttgcttgaaattgaa

SEQ ID NO: 15 - RNA, 21 nt, synthetic construct
  source 1..21: mol_type = other RNA; note = SR011543S
tccacgttgcttgaaattgaa

SEQ ID NO: 16 - RNA, 21 nt, synthetic construct
  source 1..21: mol_type = other RNA; note = SR002956S
gctcaacatatttgatcagta

SEQ ID NO: 17 - RNA, 19 nt, synthetic construct
  source 1..19: mol_type = other RNA; note = SR011283S
cacgttgcttgaaattgaa

SEQ ID NO: 18 - RNA, 21 nt, synthetic construct
  source 1..21: mol_type = other RNA; note = SR011287S
tccacgttgcttgaaattgaa

SEQ ID NO: 19 - RNA, 21 nt, synthetic construct
  source 1..21: mol_type = other RNA; note = SR011268A
ttcaatttcaagcaacgtgga

SEQ ID NO: 20 - RNA, 21 nt, synthetic construct
  source 1..21: mol_type = other RNA; note = SR002964A
tactgatcaaatatgttgagc

SEQ ID NO: 21 - RNA, 21 nt, synthetic construct
  source 1..21: mol_type = other RNA; note = SR011277S
aaaagggacagtattctcagt

SEQ ID NO: 22 - RNA, 21 nt, synthetic construct
  source 1..21: mol_type = other RNA; note = SR011278S
aaaagggacagtattctcagt

SEQ ID NO: 23 - RNA, 21 nt, synthetic construct
  source 1..21: mol_type = other RNA; note = SR011280S
aaaagggacagtattctcagt

SEQ ID NO: 24 - RNA, 21 nt, synthetic construct
  source 1..21: mol_type = other RNA; note = SR011224A
actgagaatactgtccctttt

SEQ ID NO: 25 - DNA, 18 bp, synthetic construct
  source 1..18: mol_type = other DNA; note = primer
gggaagaccgcggagtct

SEQ ID NO: 26 - DNA, 30 bp, synthetic construct
  source 1..30: mol_type = other DNA; note = primer
cagttctactctgtacactccttctacaaa

SEQ ID NO: 27 - DNA, 23 bp, synthetic construct
  source 1..23: mol_type = other DNA; note = primer
ctgcacgggctcaccacagatga

SEQ ID NO: 28 - DNA, 20 bp, synthetic construct
  source 1..20: mol_type = other DNA; note = primer
cggcaaattcaacggcacag

SEQ ID NO: 29 - DNA, 20 bp, synthetic construct
  source 1..20: mol_type = other DNA; note = primer
ccacgacatactcagcaccg

SEQ ID NO: 30 - DNA, 27 bp, synthetic construct
  source 1..27: mol_type = other DNA; note = primer
accatcttccaggagcgagaccccact